SEQUENCE LISTING (15 sequences)

SEQ ID NO: 1 - DNA, 1842 bp, Staphylococcus lugdunensis
  CDS (1)..(1839)
atgaaaaagactttcatatcactcttatccgcaacagttatactttcaggttgtggcgttggcgaacatcaaaataataattctaatcatgatgctaaaggtgtgaacacttcaaatgttaaaatcaaaaattataaccaagcatcatctgcgctgcaaatagataattcaaaatggaaatatgatagtaaaaataacgtttattatcaactaaatataagttatgtctccaatccccaagctaaaaatgtagaaaaattaggtatctatgtaccagctgcttatttcaaaggtaaaaagaatcataatgggacatataccgttactgtaaacgatgctaagaaagttaacggctattctgctagaacagcacctatcgtttatccagtcaatacacctggttatgccgaacaaagtgcacctacgtcatatcgttatagtaatatttctaagtatatgaaagctggattcatatatgttgaagcaggattacgaggacgtagtatgagcatgggcaataacagcagtaatgcatcaactaaatcatatgaaaccggttctccttggggtgtaaccgatcttaaagcagcaatcagatattaccgtttcaacgatagtagtctaccaggtaacagtagtaagatttatacttttggtcatagtggcggtggtgctcaaagtgctattgccggtgcatcaggtgatagcaagctctactataaatatttagaacaaattggcgcagccatgacagataaaaatggaaaatatatcagtgataaaattgacggtgctatggcgtggtgccctattacaagtctagatcaagccgatgctgcttatgaatggcaaatgggacaatatggtaatgaaggtaatcgcaagaaaaattcattccaaaaacaattatcaaccgatttagcatcatcttatgcaagctacttaaataaactaaatctgaaaaatggaaatactacattatcattaactaaatctaaaaatggtcaatatactgaaggctcatatgctaaatatctaaaaaaagaaattgaagattcagctacagaattcttaaataatacaacattcccttacaaacaaaatagcactgagcaagcaggcatgggtaatggtggacctagcggtggaaaaccttctggcaaaatgggatctatgcctcaaatgagaaaacaatcttcaaataaaacatacaaaacaatggatgcttacttaaaagatctaaataaaaaaggcacatggatcacgtatgataagaaaacaaaacgcgcacatattacaagtcttaaagactttgcgaaatattataaacaaccttctaaatcagtttcagcctttgatgatttaaaacgtagccaagctgaaaatgaagtgtttggaacatcaggtagtgacagtaaattacattttgatcaatcactagctaaacttttaacagaaaataaatctaactatagcaaactaaatggttggaatagtaactatgtttcatcatataaaaatgacttaacaaaaacagataaattaggcacaagcatgtcaacaagaatgaatatgtacaatccaatgtattacttatctgattactatagcgggtatggtaaatctaatgtggcaaatcattggagaattagaacaggtattcaacaaggagatacggccttaaatactgaaactaatctttcgctagctttaaaagaacgcgttggttctaaaaacgttgacttcaaaacagtttgggatcaaggtcatacaatggcagaaacatcaggtaatagtgatagtaacttcatcaaatgggtagaaagtattaataaaaaatag

SEQ ID NO: 2 - protein, 613 aa, Staphylococcus lugdunensis
MKKTFISLLSATVILSGCGVGEHQNNNSNHDAKGVNTSNVKIKNYNQASSALQIDNSKWKYDSKNNVYYQLNISYVSNPQAKNVEKLGIYVPAAYFKGKKNHNGTYTVTVNDAKKVNGYSARTAPIVYPVNTPGYAEQSAPTSYRYSNISKYMKAGFIYVEAGLRGRSMSMGNNSSNASTKSYETGSPWGVTDLKAAIRYYRFNDSSLPGNSSKIYTFGHSGGGAQSAIAGASGDSKLYYKYLEQIGAAMTDKNGKYISDKIDGAMAWCPITSLDQADAAYEWQMGQYGNEGNRKKNSFQKQLSTDLASSYASYLNKLNLKNGNTTLSLTKSKNGQYTEGSYAKYLKKEIEDSATEFLNNTTFPYKQNSTEQAGMGNGGPSGGKPSGKMGSMPQMRKQSSNKTYKTMDAYLKDLNKKGTWITYDKKTKRAHITSLKDFAKYYKQPSKSVSAFDDLKRSQAENEVFGTSGSDSKLHFDQSLAKLLTENKSNYSKLNGWNSNYVSSYKNDLTKTDKLGTSMSTRMNMYNPMYYLSDYYSGYGKSNVANHWRIRTGIQQGDTALNTETNLSLALKERVGSKNVDFKTVWDQGHTMAETSGNSDSNFIKWVESINKK

SEQ ID NO: 3 - DNA, 22 bp, Artificial
  primer
gatgtgctgcaaggcgaattgt

SEQ ID NO: 4 - DNA, 24 bp, Artificial
  primer
gctcgtatgttgtgtggaattgtg

SEQ ID NO: 5 - DNA, 6994 bp, Staphylococcus lugdunensis
aagcttgagcaacttctttgtccaatgtggtaactaatgctttattgaaataagaagtttcaatgcaaagcggcttatcgtctaaataacgaatgcgctctactaaatagacttgtgtatctttatcttttatctgtaagtgtttttgtgtttcctcgttaggcggtatgatgtcgatgttaagcactttacttgttacacgatgtccttgtaaattatctgaaaagccctttgtttttaacagattaatatatccagattttttgctatttctaacatatataccactgccccgagtttgatagataataccatctttttcaagtaactctaatgctttaacaatcgtacttttactcacttgatacgctatttttagttctgctaggcttggaagtttgtcaccaactttatatttcccatttgcaatagctgcggtaattccatgtgcaacatgttcgtactttaacatgattaatctccttaatgctgtatttacaatatagtataccaaaaataagctgaaattaaattgtaccggtataattgtataaaaacgatttacaaattgtaccggtataattataatgagttgtgtaagggtttccatatttgggataaataaaaattaaatagattgggaggttgtgttatgagtgaaagagatagaaaattagcgatacatatccttgaagccgttggtggaaaggaaaatatcgttaattacacgcattgcgcaacgcgattacgtatcgtcttaaaacgttctattccggatgctaaaaagaaagtagcaagtatggatggcgtagtaacggtagttgaaaataatggacaatttcaagtggttattggcaatgcggtcgcaagtgtttatcaagaatttgagggattattgggagaagaaaatcaacaatctagcgatgaagacagtgaaaaaggtacgattattaatcgtattattgcaacgatgtctgccgtctttgcaccatttatttatgtcttagcggctgccggtatattacaaggtatattaattattgcgaaaatgttgttttcatcatttgagaagactggcacatatgaagtatttaactttatttcatgggcaccatttacatttttaccaatatttattgcgattactgcagcaaaacatttcaaagtgaatacttatatcgcagtagcttgttgtgctgcactcgttagcccggatttagcaggaattgtagaacgcattgggagtggacatacgtttacatttattggtattccattaagtgaaacaagctatacatcatcagtgttaccaccactattattagtatgggtgttgtcatatgttgaaaaatatttaaataaatatattcatgaagttatacgcccgttatttgtaccgttcttcagtattgtaattatggtgccattaacattattaattattggtccattagcatctggggctgcacatggtattgcacaaggctttaactggctcgtagctgtagcaccatggttagcaggattattaattggtgcattttggcaggtatttgttatctttggtgttcattggggcattacgccaatggtattggcaaactttgaacagtataaaagtgatgctttccaagcctttcaaacaattgctgttattgctcaatttggtgcagttatgggtgtattcttcaaaacgagaaataaggatttaaagcgtgtatcgttatcagcagggattacaggcatctttggaattactgaaccgtctatgtatggtgtgaatttgcgctttaaaaagccattcattatcgcttgtattagtggtggtattggggctttagttgcaagtttctttaatccaaaatattatgcatatgctggtcttcctggaccgataacaattgtcaatggttatagtcctgataacatctcatcaatatggggaattttacttggttcagccgttgcaattattttacctatcattttaatccaagtacttggctatggtgaagatgtgacagaacaggtagaacgtgttgacgtcgaagaaggacatgagataaatgaacaaacacgtttgactactacatctgatcaacttgaaacagagatacatgctccactaaaaggtttagtagtagcattatctgaagtagatgatccaatatttgcggaaggtatgatgggtaaaggattagccattcagccagaaagttatgaagtgacagcacctctcaaaggggtgattagtatggttgcagtaagtaaacacgcgataggtattacatcgccagaggggatcgaacttttagttcacgttggtttagatactgtgcaattaaatggacaaggctttgaattacttgttgctgaaaatgatcaggtcgaagaagggcagcctttgctgagatttgatgcaaattatattaaagaacagggctacgaacttattacacctgtaattattactaacagtgatgaatttgaagacattatcatagctgacagtcaaaatatcacatctagagaaaaaataatgacagtaattaaaaaatagagtgtaaaggagatttaaacatggctaaattaccacaagatttcttatggggcggcgcacttgctgccaatcaatttgaaggtggctatgaccaaggtggcaaaggcttaagtgtaattgatgtaatgacagcaggcgcacatggtcaagcacgtgaaattacacaagatatagaacctgataaatattatcctaatcatatgggtattgatttttaccatcgctataaagaagatgtcgcattgtttgctgaaatgggattgaaatgtttgcgtacatcgattgcatggacacgtattttccctaatggtaatgaaacagaaccgaacgaagcaggactgcaattctatgatgatttatttgatgaattattgaaatatgacattcaaccagtgattacattatcgcattttgagatgccactacatctagcacgtgaatatggcggttttagaaaccgtaaagtagcagattattttgcatattttgctgaaactgtatttaagcgttataaagacaaagttaaatactggatgacatttaatgaaattaataaccaaatggacatcaataatcctattttcttatggacaaactctggtgtatcgttgacagatgatgataataaattagaagtattatatcaggtggcacataatgaattgattgcaagtgcgaaagcagtaaaaattggtaaggagataaatccagattttgaaattggtgctatgatttcccatgttccaatctacccatatagctgtaatccgcaagatataatggaggcagaaatcgcgagtcgtatgcgtttcttctttccagatgtacaagcacgaggttattacccaggttatgcgacaaaaatgtttgaacgtgaaggcttcgacattggttggcaagaaggagacgatgtgattctacgtgaaggtaaagtcgattatattggatttagttattatatgtctacggtcgttaaatctgataatgaggctagtgtagaagataatatagtaaatggtggcttacccaattcggttgataatccttatattaagaaaagtgattggggctgggccattgatccggttggcttacgttatacattaaatgcattatataatcgttatcaaattcctttatttatagttgaaaatggatttggtgccgtagatgagtttgatgataaaggtaaaatccatgacgaatatcgtatcgagtatttacgtgaacatattgaagcggctattgatgcagtagatcaagatggcgtagaattaatgggctatacaccttggggtattattgatattgtatcctttacgacaggtgaaatgaaaaaacgttatggtttaattcatgttgatcgtgataatgaaggaaacggttcattagaacgtaccaagaaagattcatttgactggtatcgcaaagttattgagtcaaatggggaagttttgtaaattaaaaagagagctgaacagaattcattatgaattcttagtccagctctggcaaggttgactaagattgaaaaagcatgctataagcatattttcaattgagtcaactactgccaatcattagttcacagcctaaaacatgatgatgttttaggctgtttaatacatttagctattttttattaatactttctacccatttgatgaagttactatcactattacctgatgtttctgccattgtatgaccttgatcccaaactgttttgaagtcaacgtttttagaaccaacgcgttcttttaaagctagcgaaagattagtttcagtatttaaggccgtatctccttgttgaatacctgttctaattctccaatgatttgccacattagatttaccatacccgctatagtaatcagataagtaatacattggattgtacatattcattcttgttgacatgcttgtgcctaatttatctgtttttgttaagtcatttttatatgatgaaacatagttactattccaaccatttagtttgctatagttagatttattttctgttaaaagtttagctagtgattgatcaaaatgtaatttactgtcactacctgatgttccaaacacttcattttcagcttggctacgttttaaatcatcaaaggctgaaactgatttagaaggttgtttataatatttcgcaaagtctttaagacttgtaatatgtgcgcgttttgttttcttatcatacgtgatccatgtgccttttttatttagatcttttaagtaagcatccattgttttgtatgttttatttgaagattgttttctcatttgaggcatagatcccattttgccagaaggttttccaccgctaggtccaccattacccatgcctgcttgctcagtgctattttgtttgtaagggaatgttgtattatttaagaattctgtagctgaatcttcaatttctttttttagatatttagcatatgagccttcagtatattgaccatttttagatttagttaatgataatgtagtatttccatttttcagatttagtttatttaagtagcttgcataagatgatgctaaatcggttgataattgtttttggaatgaatttttcttgcgattaccttcattaccatattgtcccatttgccattcataagcagcatcggcttgatctagacttgtaatagggcaccacgccatagcaccgtcaattttatcactgatatattttccatttttatctgtcatggctgcgccaatttgttctaaatatttatagtagagcttgctatcacctgatgcaccggcaatagcactttgagcaccaccgccactatgaccaaaagtataaatcttactactgttacctggtagactactatcgttgaaacggtaatatctgattgctgctttaagatcggttacaccccaaggagaaccggtttcatatgatttagttgatgcattactgctgttattgcccatgctcatactacgtcctcgtaatcctgcttcaacatatatgaatccagctttcatatacttagaaatattactataacgatatgacgtaggtgcactttgttcggcataaccaggtgtattgactggataaacgataggtgctgttctagcagaatagccgttaactttcttagcatcgtttacagtaacggtatatgtcccattatgattctttttacctttgaaataagcagctggtacatagatacctaatttttctacatttttagcttggggattggagacataacttatatttagttgataataaacgttatttttactatcatatttccattttgaattatctatttgcagcgcagatgatgcttggttataatttttgattttaacatttgaagtgttcacacctttagcatcatgattagaattattattttgatgttcgccaacgccacaacctgaaagtataactgttgcggataagagtgatatgaaagtctttttcatctaattagcctccttaatttgtcgtagttctattattagtcgcaaataaggagcatatcaattcggattgagcgcatgaaccattctaaattagaaagatgatgtacaccttaggtctcttgacagtatttgcttgaaattagtttctgtgctttttggctaactctagcaaatatgccacctctttagaagcaattaaatgattatgaacattaacgttaatatagagttttataggtgaattcagctgaacgatttttaaaggagacgttacgtaataattagcctgttgttccataatcaatgcgacgccattatttgtatttaccatttcaagtaaattttcaatacgaggtgctgtaaatgaaattaatggataaaattgttgttcttcacaataatttaaaaaagcttgatgctccaatgcgaactcaggtggtaaatagagttgttcattttcaagcatcgatagatcgatttgtgagacataggctaatggatgagattcatgcacaatagcacataagctttcttcaatataagtaaaagaatgattatcatgaggtggatgtggtgatcttaaaaagctaatgtcaatcttaccttcattaatcaattgagctaattcttgttctgacccttcatgaatacgaaatctaatattagaaaatttttgaacacattttgagattaagtttgtaataccatattcaatcattaatcgagaagcgccaaggttaatggtgattaaattatcattttcctgttgctcaagctgttttgataaatctttataattttggcaaatgattttggcataatgcagaaatagtttacctgattctgttagttcaaattgtttatttgcgcgatctataagacgataaccgcactcattttccaaagaaacaatgtgcttagacagtgtagattgcgctatgtagagttcttgagaagctt

SEQ ID NO: 6 - DNA, 29 bp, Artificial
  primer
gaaagaatccaatatttgcggaaggtatg

SEQ ID NO: 7 - DNA, 27 bp, Artificial
  primer
gaaagtcgacttcaaaacagtttggga

SEQ ID NO: 8 - DNA, 29 bp, Artificial
  primer
gaaagtcgactggtatcgcaaagttattg

SEQ ID NO: 9 - DNA, 29 bp, Artificial
  primer
gaaaggatccacctcatgataatcattct

SEQ ID NO: 10 - DNA, 36 bp, Artificial
  primer
gaaaggatccatgaaaaagactttcatatcactctt

SEQ ID NO: 11 - DNA, 38 bp, Artificial
  primer
ggaaaactcgagctattttttattaatactttctaccc

SEQ ID NO: 12 - DNA, 23 bp, Artificial
  primer
gggctggcaagccacgtttggtg

SEQ ID NO: 13 - DNA, 23 bp, Artificial
  primer
ccgggagctgcatgtgtcagagg

SEQ ID NO: 14 - DNA, 20 bp, Artificial
  primer
cgtggtgccctattacaagt

SEQ ID NO: 15 - DNA, 22 bp, Artificial
  primer
tgagccttcagtatattgacca

(19) United States
(12) Patent Application Publication
(10) Pub. No.: US 2009/0124564 A1
(43) Pub. Date: May 14, 2009

(54) Compositions and methods for regulating apoptosis

(75) Inventors: Atan Gross, Rechovot (IL); Michal Grinberg, Rechovot (IL); Yehudit Zaltsman, Rishon-LeZion (IL)

Correspondence Address: MARTIN D. MOYNIHAN d/b/a PRTSI, INC., P.O. BOX 16446, ARLINGTON, VA, 22215, US

(73) Assignee: Yeda Research and Development Co. Ltd., Rechovot (IL)

(21) Appl. No.: 11/794,783

(22) PCT Filed: Jan. 5, 2006
(86) PCT No.: PCT/IL06/00021

§ 371 Date: Nov. 3, 2008

Related U.S. Application Data
(60) Provisional application No. 60/641,424, filed on Jan. 6, 2005.

(51) Int. Cl. A61K 31/7052; C12N 5/00; A01K 67/033; A61P 35/00; A61P 37/02; A61P 25/00

(52) U.S. Cl. 514/44; 435/375; 800/13

(57) ABSTRACT

Methods and pharmaceutical compositions for regulating apoptosis and treating pathologies associated with disregulated apoptosis are provided. Specifically the present invention provides agents capable of modulating the expression of an MCD-containing protein (e.g., Mtch2) capable of tBID binding activity and/or the tBID binding activity of the MCD-containing protein.

### FIELD AND BACKGROUND OF THE INVENTION

The present invention relates to methods of regulating apoptosis. More particularly, the present invention relates to methods of regulating apoptosis by modulating the expression level or activity of molecules capable of binding to tBID such as Mtch2.

Apoptosis, or programmed cell death, is a naturally occurring form of cell suicide playing a crucial role in ensuring the normal development and maintenance of cells, organs, and tissues. In particular, apoptosis plays an essential role in the maintenance of health by enabling the strictly regulated, orderly elimination of damaged cells, such as senescent, mutated or hyperproliferative cells, in a physiologically beneficial way, in contrast to highly pathogenic uncontrolled process of necrotic cell death. Disregulation of this beneficial and natural process, however, is involved in the pathogenesis of numerous lethal and/or highly debilitating diseases, such as cancer and autoimmune disease, for which no satisfactory therapy exists. Excessive apoptosis is associated with pathological cell loss associated with degenerative disorders. Such degenerative disorders include neurological disorders such as Alzheimer's disease, Parkinson's disease, amyotrophic lateral sclerosis (ALS) and retinitis pigmentosa. Excessive apoptosis is also associated with hematological disorders and viral infections including human immunodeficiency virus (HIV)-induced acquired immunodeficiency syndrome (AIDS). Stimuli which can trigger excessive apoptosis include growth factors such as tumor necrosis factor (TNF), Fas, and transforming growth factor (TGF)-β; neurotransmitters; growth factor withdrawal; loss of extracellular matrix attachment; and excessive fluctuations in intracellular calcium levels. Alternatively, insufficient or failure of apoptosis, which may be triggered by various growth factors, extracellular matrix changes, CD40 ligand, viral gene products, neutral amino acids, zinc, estrogen and androgens, is a major contributor to the pathogenesis of cancer, autoimmune disorders, bacterial infections, and viral infections. Disregulated apoptosis is also associated with the pathogenesis of age-related disorders such as osteoporosis and atherosclerosis, and normal apoptosis is a key mediator of the process of aging itself.

Hence, methods of regulating apoptosis are highly desirable since they could be exploited to prevent, treat or control diseases, disorders or conditions associated with disregulated, excessive, or insufficient apoptosis.

Apoptosis is regulated by proteins which function to promote or inhibit programmed cell death. Several proteins which modulate the apoptotic process have now been identified, including members of BCL-2 family which are major regulators of the apoptotic process. The precise cell death-inducing mechanisms of these proteins are unknown, although it has been suggested that their function may depend on their ability to modulate mitochondrial function (Gross, A., 2001. IUBMB Life 52:231-236). The BCL-2 family is comprised of both pro-apoptotic proteins, such as BAX, as well as anti-apoptotic proteins, such as BCL-2. Most BCL-2 family members share three conserved domains; BH1, BH2, and BH3, of which BH3 functions as a death domain in the pro-apoptotic members of the family. One subset of such pro-apoptotic molecules comprises the BH3-only proteins (i.e., proteins which contain only the BH3 domain out of the three conserved BH domains) including the major regulator of apoptosis BID (BH3 interacting death agonist). Deletion and mutagenesis analysis studies have argued that the amphipathic α-helical BH3 domain serves as a critical death domain in these proteins. Another major component of the programmed cell death machinery is a proteolytic system involving the caspase family of cysteine proteases.

Execution of apoptosis involves two major biochemical pathways of caspase activation, one triggered via cell surface receptors (i.e., an extrinsic pathway), and the other resulting from mitochondrial events (i.e. an intrinsic pathway). The cell-intrinsic apoptotic pathway involves the activation of pro-apoptotic BCL-2 family members, which induce the permeabilization of the outer mitochondrial membrane (OMM), resulting in the release of cytochrome c (Cyt c) and other inter membrane space (IMS) proteins. In the cytosol, Cyt c induces the oligomerization of Apaf-1, which recruits and activates caspase-9. Caspase-9 activates caspase-3, which leads to apoptotic cell death.

In the extrinsic pathway, apoptosis is initiated through activation of certain cell surface receptors. The best characterized are members of the TNF/Fas receptor family. These receptors utilize protein interaction modules known as death domains, and death effector domains (DEDs) to assemble the death-inducing signaling complex (DISC) which recruits and activates caspase-8. Active caspase-8 can initiate both the activation of a cascade of caspases and the cleavage of BID. Cleavage of cytosolic BID at Asp59 yields a p15 C-terminal truncated fragment (tBID) that translocates to the mitochondria. Targeting of tBID to mitochondria induces the activation of BAX and BAK in a BH3-dependent manner, resulting in the release of Cyt c. The requirement for BID in the extrinsic death pathway was demonstrated in Bid-deficient mice, which were resistant to Fas and TNFα-induced hepatocellular apoptosis (Yin, X. M., et al., 1999, Nature, 400: 886-891; Zhao, Y., et al., 2001, J. Biol. Chem. 276: 27432-27440). In addition, the presence of either BAX or BAK is necessary for the action of tBID since Bax-Bak double-deficient mouse embryonic fibroblasts (MEFs) are resistant to tBID-induced apoptosis and several other BH3-only molecules [Zong W X, 2001, Genes Dev, 15: 1481-1486; Wei, M. C. 2001 Science 292: 727-730].

Activation of BAX and BAK involves their oligomerization and membrane integration. Since recombinant BAX was shown to be capable of forming channels in artificial membranes large enough to release Cyt c in-vitro, it has been proposed that tBID activates BAX/BAK to form non-selective channels in the OMM for the release of IMS proteins such as Cyt c. However this simple model cannot account for the rapid kinetics and complete extent of Cyt c release during apoptosis, since the majority of Cyt c is buried in intramitochondrial cristae. High-voltage electron microscopic (HVEM) tomography of mitochondria has revealed that the IMS is very narrow, consistent with functional estimates that only 15-20% of total Cyt c is available in the IMS. Activation of the permeability transition pore (PTP), a high-conductance inner mitochondrial membrane (IMM) channel, that ultimately leads to mitochondrial swelling with secondary rupture of the OMM can lead to the release of these compartmentalized stores of Cyt c. However, this is not the case with tBID since it does not induce detectable swelling of mitochondria.

Recently it was demonstrated that tBID causes the release of the compartmentalized stores of Cyt c (˜85%) by inducing reorganization of the IMM. Interestingly, this reorganization did not require tBID's BH3 domain and was independent of BAX and BAK, but was dependent on the transient opening of the PTP, which does not lead to swelling of the mitochondria. Thus, tBID is involved in two distinct pathways to induce Cyt c release: one, which is BH3/BAX/BAK-dependent and mediates the release of Cyt c across the OMM, and another, which is BH3/BAX/BAK-independent and responsible for the redistribution of Cyt c stored in intramitochondrial cristae (see FIG. 17).

Cyt c mobilization might not be the only result of IMM reorganization, since tBID was reported to facilitate Ca²⁺ delivery to the mitochondria, possibly through releasing a negative modulator of the sites that mediate Ca²⁺ uptake through the IMM. In this respect, it was demonstrated that early in apoptosis, Cyt c translocates to the endoplasmic reticulum to bind inositol triphosphate receptors (InsP3R), resulting in cytosolic Ca²⁺ increases, which are linked to the coordinated release of Cyt c from all mitochondria. Thus, Cyt c-InsP3R interactions provide a molecular mechanism whereby the endoplasmic reticulum and mitochondria work in cooperate to execute apoptosis.

Altogether, the prior art data demonstrate that tBID is a critical mediator of apoptosis.

In a recent study, a stapled BH3 peptide [also called “stabilized alpha-helix of BCL-2 domain” (SAHB)], which mimics the apoptotic action of BID, specifically activated the apoptotic pathway to kill leukemia cells (Walensky L. D., et al., 2004, Science, 305: 1466-1470 and U.S. Pat. Appl. No. 20050250680 to Walensky, Loren D. et al.).

An attractive approach to regulate apoptosis involves modulation of BID-interacting proteins. These include the BAK (Wei M C, 2000, Genes Dev, 14: 2060-2071) and BAX (Eskes, R. et al., 2000. Mol Cell Biol. 20:929-935) which are induced to homoligomerize by tBID during apoptosis; the mitochondrial permeability transition pore (mPTP) which was proposed to mediate tBID induced remodeling of mitochondrial structure thereby leading to redistribution of Cyt c stored in intra-mitochondrial cristae (Scorrano, L. et al., 2002. Dev Cell. 2:55-67), and the BCL-2 molecule, which was suggested to interact with tBID (Cheng, E. H. et al., 2001. Mol. Cell. 8:705-711).

However, to date, modulation of the expression levels or activity of BID-interacting molecules to optimally regulate apoptosis have not been demonstrated.

### SUMMARY OF THE INVENTION

According to one aspect of the present invention there is provided a method of regulating apoptosis in a cell, the method comprising contacting the cell with an agent capable of modulating: (i) an expression of an MCD-containing protein capable of tBID binding activity; and/or (ii) the tBID binding activity of the MCD-containing protein, thereby regulating apoptosis in the cell.

According to another aspect of the present invention there is provided a method of treating a pathology associated with disregulated apoptosis in a subject comprising administering to the subject an agent capable of modulating: (i) an expression of an MCD-containing protein capable of tBID binding activity; and/or (ii) the tBID binding activity of the MCD-containing protein, thereby treating the pathology.

According to yet another aspect of the present invention there is provided a pharmaceutical composition for regulating apoptosis comprising an agent capable of modulating: (i) an expression of an MCD-containing protein capable of tBID binding activity; and/or (ii) the tBID binding activity of the MCD-containing protein, and a pharmaceutical acceptable carrier.

According to still another aspect of the present invention there is provided a multicellular organism comprising a genome which comprises a genetically modified Mtch2 gene being incapable of encoding a functional Mtch2 protein.

According to further features in preferred embodiments of the invention described below, regulating apoptosis is upregulating apoptosis.

According to still further features in the described preferred embodiments the agent is capable of decreasing the expression of the MCD-containing protein.

According to still further features in the described preferred embodiments the agent capable of decreasing the expression of the MCD-containing protein is selected from the group consisting of an antibody, an siRNA, an antisense oligonucleotide, a DNAzyme, and a Ribozyme.

According to still further features in the described preferred embodiments the siRNA is as set forth in SEQ ID NO:27.

According to still further features in the described preferred embodiments the agent is capable of increasing the tBID binding activity of the MCD-containing protein.

According to still further features in the described preferred embodiments the regulating apoptosis is downregulating apoptosis.

According to still further features in the described preferred embodiments the agent is capable of increasing the expression of the MCD-containing protein.

According to still further features in the described preferred embodiments the agent is selected from the group consisting of: (i) an exogenous polynucleotide encoding at least a functional portion of the MCD-containing protein, and (ii) an exogenous polypeptide including the at least a functional portion of the MCD-containing protein.

According to still further features in the described preferred embodiments the agent is capable of decreasing the tBID binding activity of the MCD-containing protein.

According to still further features in the described preferred embodiments the agent is an antibody or a peptide.

According to still further features in the described preferred embodiments the MCD-containing protein is Mtch2.

According to still further features in the described preferred embodiments the Mtch2 is a human Mtch2 as set forth by SEQ ID NO:7.

According to still further features in the described preferred embodiments the Mtch2 is selected from the group consisting of bovine Mtch2 (SEQ ID NO:15), mouse Mtch2 (SEQ ID NO:17), chicken Mtch2 (SEQ ID NO:14), xenla Mtch2 (SEQ ID NO:18) and danre Mtch2 (SEQ ID NO:16).

According to still further features in the described preferred embodiments the MCD-containing protein is selected from the group consisting of human Mtch1 (SEQ ID NO:6), drome MtchA (SEQ ID NO:22), drome MtchB (SEQ ID NO:23), Caeel Mtch (SEQ ID NO:19), anoga MtchA (SEQ ID NO:20) and bommo MtchA (SEQ ID NO:21).

According to still further features in the described preferred embodiments the disregulated apoptosis is characterized by abnormally low level of apoptosis and whereas the agent is capable of upregulating apoptosis.

According to still further features in the described preferred embodiments the disregulated apoptosis is characterized by abnormally high level of apoptosis and whereas the agent is capable downregulating apoptosis.

According to still further features in the described preferred embodiments the genetically modified Mtch2 gene in the genome is induced postnatally.

According to still further features in the described preferred embodiments an integration of the genetically modified Mtch2 gene in the genome is induced in an adult animal.

According to still further features in the described preferred embodiments the genetically modified Mtch2 gene comprises a deleted Mtch2 gene.

According to still further features in the described preferred embodiments the multicellular organism is a mouse.

The present invention successfully addresses the shortcomings of the presently known configurations by providing methods and pharmaceutical compositions for regulating apoptosis and treating pathologies associated with disregulated apoptosis.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is Western blot analysis depicting BID expression in TNFα-activated FL5.12 cells. FL5.12 cells were induced for 5 hours with TNFα and cyclohexamide [TNFα/CHX (+); lanes 2, 4, 5] or remained uninduced [TNFα/CHX (−); lanes 1, 3], following which the cytosol (cyto; lane 5) and mitochondrial membranes (lanes 1-4) were suspended in isotonic buffer and were either treated with the sulfo-BSOCOES cross-linker (+) (lanes 3, 4, 5) or with DMSO as a control (−) (lanes 1, 2). Note the presence of a 45 kDa BID-containing complex in the mitochondrial membrane fraction of TNFα/CHX —induced FL5.12 cells. IB=immunblotting.

FIGS. 2a-b are tBID Western blot analyses of mouse liver mitochondria (FIG. 2a) and Bax, Bak double knockout (DKO) mouse embryonic fibroblasts (MEFs, FIG. 2b). FIG. 2a-Mouse liver mitochondria were pre-treated for 20 minutes with proteinase K, and then incubated with HA-tBID as described under General Materials and Experimental Methods of the Examples section which follows. At the end of the reaction, mitochondria were treated with the sulfo-BSOCOES cross-linker, lysed, and equal amounts of protein (20 μg per lane) were subjected to SDS-PAGE, followed by Western blot analysis using anti-BID antibodies. Lane 1—untreated mouse liver mitochondria, lane 2—proteinase K treated mouse liver mitochondria; Note the novel, BID-immunoreactive protein species of about 35 kDa (marked with an asterisk) appearing as a result of proteinase K pre-treatment, demonstrating that the formation of the 45 kDa tBID complex requires a ˜30 kDa surface-exposed mitochondrial protein. FIG. 2b-Bax, Bak DKO MEFs were treated for 3 hours with tBID adenoviruses, following which the mitochondrial-enriched fraction was treated with either sulfo-BSOCOES (+) (lane 2) or with DMSO (−) (lane 1), lysed, and analyzed as described in FIG. 2a. Note the presence of a 45 kDa tBID reactive band in MEFs derived from Bax and Bak DKO mice demonstrating that the formation of the 45 kDa tBID-containing complex is independent of Bax and Bak presence. MW=molecular weight size marker in kDa.

FIGS. 3a-c are Coomassie blue gel staining (FIG. 3a), HA-tBID Western blot (FIG. 3b) and amino acid sequences (FIG. 3c) demonstrating that HA-tBID forms a ˜45 kDa complex with Mtch2, a previously uncharacterized 33 kDa mitochondrial protein. FIG. 3a-Coomassie blue staining of a gel loaded with large scale protein purification of the tBID cross-linked complex from 293T cells transfected with HA-tBID. The eluted material from the anti-HA antibody affinity column was loaded onto a single lane, and separated by SDS-PAGE followed by Coomassie blue staining. The top arrow marks the band suspected to be the 45 kDa band that was cut out of the gel and analyzed by mass spectrometry. The bottom arrow marks the expected size of HA-tBID. FIG. 3b-tBID Western blot analysis of the large scale tBID cross-linked complex. A small portion of the eluted material was also taken for Western blot analysis using anti-HA Abs. Note the 45 kDa tBID-reactive band and the additional BID immunoreactive bands (marked with asterisk) possibly representing additional tBID cross-linked complexes. MW=molecular weight size marker in kDa. FIG. 3c-sequence diagrams depicting the sequence of mouse tBID (SEQ ID NO:8) with the two mass spectroscopy-identified tBID-derived peptides (SEQ ID NOs:3 and 4; shown in bold and underlined) and the sequence of human Mtch2 (SEQ ID NO:7) with the mass spectroscopy-identified Mtch2-derived peptide (SEQ ID NO:5; shown in bold and underlined) resulting from the large scale purified 45 kDa complex.

FIGS. 4a-b depict the Mtch2 RT-PCR product (FIG. 4a) and recombinant protein (FIG. 4b). FIG. 4a-a UV fluorescence photograph of an ethidium bromide-stained agarose gel depicting RT-PCR amplification of Mtch2. Total RNA was prepared from HeLa cells and RT-PCR amplification was performed using the forward (SEQ ID NO:1) and reverse (SEQ ID NO:2) PCR primers. Lane 1-RT-PCR product, lane 2—molecular weight marker (100 bp ladder). Note the presence of a 912 bp RT-PCR product corresponding to the Mtch2 cDNA; FIG. 4b-a photograph of a chemiluminescence-developed Western blot analysis depicting expression of recombinant myc- and His-tagged Mtch2 fusion protein. The Mtch2 cDNA product was cloned into pcDNAIII.1-myc-His (Invitrogen), and transiently transfected into 293T cells. Eighteen hours post-transfection, cells were harvested, lysed and analyzed by Western blot using anti-myc antibodies. Lane 1-non-transfected (−) 293T cells, lane 2—transfected (+) 293T cells. Note the presence of the 36 kDa band corresponding to the recombinant myc- and His-tagged Mtch2 fusion protein (Mtch2-MH; marked by an arrow).

FIGS. 5a-c are Western blot analyses depicting the interaction of tBID and Mtch2-MH at the mitochondrial membrane. 293T-T-Rex cells were transiently transfected with pCA14-HA-tBID to generate cell line T-Rex-HA-tBID for tetracycline-inducible expression of HA-tBID alone, and T-Rex-HA-tBID cells were transfected with pcDNAIII.1-Mtch2-myc-His for co-expression of Mtch2-MH and tetracycline-inducible HA-tBID. At 18 hours post-transfection, transfectants were treated for 5 hours with doxycyclin to induce expression of HA-tBID. Mitochondria prepared from these cells were treated with the sulfo-BSOCOES cross-linker and lysed, and the lysate proteins were either subjected to Western blot analysis using the anti HA antibody (FIG. 5a) or were loaded onto nickel columns and the eluted proteins were further subjected to Western blot analyses using the anti myc (FIG. 5b) or anti-HA (FIG. 5c) antibodies. Lane 1—Tetracycline (doxycyclin)-induced T-Rex-HA-tBID expressing recombinant HA-tBID; lane 2—Tetracycline—induced T-Rex-HA-tBID cells co-transfected with pcDNAIII.1-Mtch2-myc-His and expressing recombinant HA-tBID and Mtch2-MH. Note the 48 kDa protein species (marked with an asterisk, lane 2 of FIGS. 5a and 5b) representing the HA-tBID/Mtch2-MH complex and the 36 kDa protein species (marked with two asterisks, lane 2 of FIG. 5b) representing the Mtch2-MH protein.

FIGS. 6a-b are Western blot analyses of Mtch2 and tBID—expressing 293T cells following proteinase K (FIG. 6a) or urea (FIG. 6b) treatments. 293T-T-Rex cells were transiently transfected with pCA14-HA-tBID to generate cell line T-Rex-HA-tBID for tetracycline-inducible expression of tBID alone, and T-Rex-HA-tBID cells were transfected with pcDNAIII.1-Mtch2-myc-His for co-expression of Mtch2-MH and tetracycline-inducible HA-tBID. At 18 hours post-transfection, transfectants were treated for 5 hours with doxycyclin to induce expression of HA-tBID. FIG. 6a-Mtch2 Western blot analysis following proteinase K treatment. Mitochondria prepared from the transfected cells were treated with protease K or remained untreated. At the end of the reaction, mitochondria were lysed, size fractionated by SDS-PAGE and analyzed by Western blot using anti-myc Abs (capable of detecting Mtch2-myc-His). Note that following proteinase K treatment the intensity of the ˜35 kDa Mtch2 band is decreased (compare lane 2 and 1 and lane 4 and 3) and a new ˜25 kDa Mtch2 band appears (marked with an asterisk; lanes 2 and 4). FIG. 6b-Mtch2 Western blot analysis following urea treatment. Mitochondria from the transfected cells were treated with 8 M urea or remained untreated and following treatment, the membranes were separated from the soluble fraction by centrifugation and proteins of both urea-insoluble [pellet; (P)] and—soluble [supernatant; (S)] fractions were analyzed by Western blot using anti-myc Abs. Note that in Mtch2 alone—expressing cells the intensity of the Mtch2 band in the urea-insoluble fraction is higher than in the urea-soluble fraction (compare lanes 1 and 2). On the other hand, note that in Mtch2-BID—expressing cells the intensity of the Mtch2 band in the urea-insoluble fraction is lower than in the urea-soluble fraction (compare lanes 3 and 4), demonstrating that the presence of tBID at the mitochondria destabilizes the membrane-attachment of Mtch2-MH.

FIG. 7a is a multiple sequence alignment diagram depicting the similarity of the amino acid sequence of the mitochondrial carrier protein domain (MCD) of human Mtch2 with those of its most closely related and functionally characterized mitochondrial carrier protein (MCP) family members. The amino acid sequence of human Mtch2 (GenBank Accession No. NP_{—}055157; SEQ ID NO:7) was searched against the NCBI Conserved Domain Database (CDD) version 1.60 using the RPS-BLAST program (Altschul, S. F. et al., 1997. Nucleic Acids Res. 25:3389-3402). Positions 128-192 of human Mtch2 were similar to the mitochondrial carrier protein domain (CDD|9082), with an e-value of 2-8 (53.8 bits). Uppercase letters denote aligned regions in the query and domain multiple alignment. The two transmembrane regions predicted by the PHD server (Rost, B. et al., 1996. Protein Sci. 5:1704-1718) are underlined in human Mtch2. Below the human Mtch2 sequence are the consensus and multiple alignments of the mitochondrial carrier domains (MCDs) taken from the two proteins with known function which were most similar to human Mtch2. The conserved MCD is marked by a box. The NCBI GenBank Accession Nos. for the aligned proteins are as follows: human Mtch2-NP_{—}055157 (Mtch2_human; gi:7657347); human mitochondrial carnitine/acylcarnitine translocase (CAC)-043772 (CAC_human; gi:3914023; SEQ ID NO:9); and human mitochondrial uncoupling protein 1 (UCP1; Thermogenin)—P25874 (UCP1_human; gi: 1351353; SEQ ID NO:13).

FIG. 7b—A graphic display of the topology of the MCP family members in the inner membrane of mitochondria. Shown are the three MCDs (marked by boxes) characteristics of all MCP family members, each composed of two transmembrane regions connected by a linker region.

FIG. 7c is a multiple sequence alignment diagram depicting amino acid sequence homology between human Mtch2 and closely related Mtch-family proteins. Residues in uppercase letters are confidently aligned. The three transmembrane regions predicted by the PHD server (Rost, B. et al., 1996. Protein Sci. 5:1704-1718) are marked by red boxes (also shown in FIG. 3c of Grinberg et al., 2005, Mol. Cell. Biol. 25: 4579-90). The single MCD is marked by a black box. The peptides used to generate the antibodies [amino acids 93-106 (Ab1), 110-127 (Ab2) and 274-288 (Ab3) as set forth in SEQ ID NO:17 (mouse Mtch2)] appear in blue boxes. NCBI GenBank Accession Nos. for the aligned proteins and their percent identity to human Mtch2 are as follows: human Mtch2—NP_{—}055157 (SEQ ID NO:7; Mtch2_human); bovine Mtch2-BAA95942 (gi:7959093; Mtch2_cow; SEQ ID NO:15; 94% identity); mouse Mtch2-AAD52647 (gi:5815347; SEQ ID NO:17; Mtch2_mouse; 93% identity); chicken (chick) Mtch2-BAA86900 (gi:6448447; SEQ ID NO:14; Mtch2_chick; 79% identity); Xenopus laevis (xenla) Mtch2-BAB03397 (gi:16326341; SEQ ID NO:18; Mtch2_xenla; 70% identity); zebrafish (Danio rerio) Mtch2—NP_{—}571457 (gi:18859045; SEQ ID NO:16; Mtch2_danre; 69% identity); human Mtch1-AAF12793 (gi:6995989; SEQ ID NO:6; 48% identity); Drosophila melanogaster (drome) MtchA-AAD52649 (gi:5815351; SEQ ID NO:22; 38% identity); Drosophila melanogaster (drome) MtchB-AAM48309 (gi:21391910; SEQ ID NO:23; 34% identity); Caenorhabditis elegans (caeel) Mtch-NP_{—}495545 (gi:17533979; SEQ ID NO:19; 28% identity); Anopheles gambiae str. PEST (anoga) MtchA-EAA04685 (gi:21292540; SEQ ID NO:20; 36% identity); Bombyx mori (bommo) MtchA-BAA86901 (gi:6448449; SEQ ID NO:21; 37% identity).

FIG. 7d is a sequence alignment diagram depicting the portion of human Mtch1 (SEQ ID NO:6; Mtch1_human) and the portion of human Mtch2 protein (SEQ ID NO:7; Mtch2_human) displaying significant amino acid sequence similarity. Lower case regions are not confidently aligned. Vertical lines (|) depict identical amino acids; (+) depict similar amino acids.

FIGS. 8a-i are images of confocal microscopy depicting Mtch2 and tBID co-localization at the mitochondria. FIGS. 8a-c-HeLa cells transfected with human Mtch2-GFP were stained with Mitotracker red (MTR), fixed and analyzed using a confocal fluorescence microscope. FIG. 8a-Mtch2-GFP (green); FIG. 8b—mitochondria stained with MTR (red); FIG. 8c-a merged image of FIGS. 8a and b. FIG. 8d-f-HeLa cells transfected with mouse tBID in the presence of the broad caspase inhibitor zVAD-fmk (50 μM) were prestained with MTR, fixed, immunostained with anti-BID Abs, and analyzed as above. FIG. 8d-tBID (green); FIG. 8e-mitochondria stained with MTR (red); FIG. 8f-a merged image of FIGS. 8d and f. FIGS. 8g-i—HeLa cells transfected with both human Mtch2-GFP and mouse tBID were fixed, immunostained with anti-BID Abs, and analyzed as above. FIG. 8g-Mtch2-GFP (green); FIG. 8h-tBID (red); FIG. 8i-a merged image of FIGS. 8g and h.

FIGS. 9a-g are Western blot analyses demonstrating that Mtch2 is an integral membrane protein exposed on the surface of mitochondria. FIGS. 9a-c-Western blot analyses of 293T cells or mouse liver mitochondria using the new anti-Mtch2 antibodies (Ab1, FIG. 9a; Ab2, FIG. 9b; Ab3, FIG. 9c) raised against the corresponding peptides described in FIG. 7c. Lane 1—untreated 293 cells (293-N/T), lane 2-293T cells transfected with Mtch2-MH (293+Mtch2-MH), lane 3-mouse liver mitochondria (Liver mito). Note the ˜33 kDa band corresponding to Mtch2 protein and the higher molecular weight species (marked with an asterisk) representing cross-reactive bands.

FIG. 9d-Western blot analysis of mouse liver mitochondria using the Ab2 anti-Mtch2 Ab. Mouse liver mitochondria were treated with either 0.1 M Na₂CO₃ (pH 11.5; lane 2), or 8 M urea (Ur; lane 3) or were remained untreated (N/T; lane 1) following which the membranes were separated from the soluble fraction by centrifugation, and the membrane fractions were size-fractionated by SDS-PAGE. Note the presence of a ˜33 kDa protein band in all lanes, demonstrating that Mtch2 is an integral membrane protein.

FIGS. 9e-g-Western blot analyses of mouse liver mitochondria using the Ab1 (FIG. 9e), Ab2 (FIG. 9f) and Ab3 (FIG. 9g) anti-Mtch2 antibodies. Mouse liver mitochondria were treated with either a low (0.1 μg/ml; +) (lane 2) or high concentration (1 μg/ml; ++) (lane 3) of proteinase K (Prot K), or were remained untreated (−) (lane 1), following which the mitochondria were lysed and size-fractionated by SDS-PAGE. Note the presence of a ˜20 kDa (marked with two asterisks) and ˜10 kDa (marked with an asterisk) protein fragments depicting proteinase K—digested Mtch2 fragments.

FIG. 10 is a graphic display of the predicted topology of Mtch2 in the outer membrane of mitochondria. The MCD is marked by a box, and the “Ab” labeling indicates the location of the peptides in Mtch2 that were used for generating Ab1, Ab2, and Ab3 antibodies. The predicted proteinase K cleavage sites are marked by arrows, and the resulting three fragments are labeled as follows: N stands for the ˜10 kDa N-terminal fragment; M stands for the ˜12 kDa middle fragment; and C stands for the ˜9 kDa C-terminal fragment. IMS=inter membrane space; OMM=outer mitochondrial membrane; Cyt—cytosol.

FIG. 11 is a Western blot analysis of mouse liver mitochondria using the anti HA antibody (directed against HA-tBID). Mouse liver mitochondria were pre-treated with either a low (+) 0.1 μg/ml (lanes 3, 4) or high concentration (++) 1 μg/ml (lanes 5, 6) of proteinase K, or were remained untreated (−) (lanes 1, 2), following which they were incubated with HA-tBID. At the end of the reaction, mitochondria were treated with the BSOCOES cross-linker (lanes 2, 4, and 6) or remained untreated (lanes 1, 3, and 5), lysed, and equal amounts of protein (20 μg per lane) were size-fractionated by SDS-PAGE. Note the ˜35 kDa tBID-Mtch2 complex (marked with an asterisk) and the ˜24 kDa tBID-Mtch2 complex (marked with two asterisks) depicting the interaction of tBID with the C fragment of Mtch2.

FIGS. 12a-c are Western blot analyses of mitochondria from FL5.12 cells using anti-BID (FIGS. 12a-b) or anti-Mtch2 (FIG. 12c) antibodies. FIG. 12a—Mitochondria from FL5.12 cells, which were induced for 5 hours with TNFα/CHX, were treated with the cross-linker BSOCOES (+) (lanes 3 and 4) or were remained untreated (−) (lanes 1 and 2), following which the mitochondria were incubated in neutral (pH 7; lanes 1 and 3) or alkaline (pH 11; lanes 2 and 4) conditions. At the end of the incubation, the membranes were lysed and size-fractionated by SDS-PAGE. Note the decrease in the intensity of the band representing the 45 kDa complex under alkali conditions (lane 4) as compared to neutral conditions (lane 3) in the BSOCOES treated mitochondria. FIGS. 12b-c—The experiment presented in FIG. 12a was repeated and the gel sections corresponding to the position of the 45 kDa complex in lanes 1 and 3 of FIG. 12a (marked by boxes) were excised, and incubated in either neutral or alkaline conditions. The sections were then layered onto a 1.5% denaturing gel, the proteins were resolved by SDS-PAGE, and immunoblotted using either the anti-BID Abs (FIG. 12b) or the anti-Mtch2/Ab2 Abs (FIG. 12c). Note the 14 kDa band in lane 4 of FIG. 12b (tBID, marked with an arrow) and the 33 kDa band in lane 4 of FIG. 12c (Mtch2, marked with an arrow) demonstrating that the 45 kDa band formed in TNFα-treated FL5.12 cells represents a complex between tBID and Mtch2. The asterisk (in FIG. 12b) marks an additional BID immunoreactive band that might represent a modified form of tBID. The two asterisks (in FIG. 12b) mark cross-reactive substances that might have been released from the gel sections.

FIGS. 13a-c depict Coomassie blue staining (FIG. 13a) and Western blot analyses using α-Mtch2 (using the Ab2 antibodies) (FIG. 13b) or α-BID (FIG. 13c) antibodies of mitochondria prepared from TNFα/CHX induced FL5.12 cells on two-dimension gel (2-D-gel). FL5.12 cells were induced for 5 hours with TNFα/CHX following which the mitochondria were prepared and solubilized with 0.3% Dodecyl Maltoside (DM). DM solubilized protein complexes were resolved on a 5-13% gradient blue-native (BN) gel (1^{st} D BN-PAGE). Lanes from the BN gel were layered onto an 8-20% gradient denaturing gel and the proteins were resolved by SDS-PAGE, stained with Coomassie blue and transferred to nitrocellulose. DM-ext (FIG. 13a, right lane) marks DM solubilized protein complexes directly loaded onto the SDS-gel. Note the co-migrating positions (positioned along a dashed line which is marked with an asterisk) of Mtch2 and tBID (spot No. 1) demonstrating that the Mtch2 and tBID reside in an approximately 185 kDa resident mitochondrial complex.

FIGS. 14a-f are Western blot analyses of mitochondria from TNFα/CHX —induced FL5.12 cells separated on 2-D-gel using anti-BID (FIGS. 14a-b), anti-BAX (FIGS. 14c-d) and anti-Mtch2 (FIGS. 14e-f) antibodies. FL5.12 cells were induced for 5 hours with TNFα/CHX (+) (FIGS. 14b, d, or f) or were remained uninduced (−) (FIGS. 14a, c and e), following which mitochondria were prepared from the cells, and processed as described in FIGS. 13a-c. The left line (marked with one asterisk) is placed in the same position in which it is placed in FIGS. 13a-c. Note the co-migrating positions of tBID, BAX and Mtch2, demonstrating that activation by TNFα recruits BAX and tBID to the Mtch2-resident complex. The right line (marked with two asterisks) marks the position of BAX in viable FL5.12 cells.

FIGS. 15a-g are BID (FIG. 15a), BAX (FIGS. 15b-c), Mtch2 (FIGS. 15d-e), and αBCL-X_{L} (FIGS. 15f-g) Western blot analyses of 2-D-gel of mitochondria prepared from FL5.12 cells that stably express BCL-X_{L} (FL5.12-BCL-X_{L}). FL5.12-BCL-X_{L} cells were induced for 5 hours with TNFα/CHX (+) (FIGS. 15a, c, e, g) or were remained un-induced (−) (FIGS. 15b, d, f), following which mitochondria were prepared from the cells and processed as described in FIGS. 13a-c. The two lines (the left one marked with one asterisk and the right one marked with two asterisks) are placed in the same position in which they are placed in FIGS. 14a-f. Note the absence of BID and BAX from the approximately 185 kDa resident mitochondrial complex which contains Mtch2, demonstrating that BCL-X_{L} partially inhibits the recruitment of both BAX and tBID to the Mtch2-resident complex.

FIGS. 16a-d are Western blot analyses of TNFα-treated FL5.12-BCL-X_{L} cells using anti-BID Abs (FIGS. 16a, b), anti-BCL-X_{L} Abs (FIG. 16c), or anti-Mtch2 Abs (FIG. 16d). FIG. 16a-FL5.12-BCL-X_{L} cells were induced for 5 hours in the presence TNFα/CHX (lanes 3 and 4) or were remained un-induced (lanes 1 and 2), following which mitochondria were prepared from the cells and were treated with the BSOCOES cross-linker (+) (lanes 2 and 4) or remained untreated (−) (lanes 1 and 3). At the end of the incubation, the membranes were lysed and were size-fractionated by SDS-PAGE and immunoblotted using anti-BID Abs. FIGS. 16b-d—The experiment presented in FIG. 16a was repeated and the gel sections corresponding to the position of the 45 kDa band in lanes 3 and 4 (FIG. 16a; marked by boxes) were excised, incubated in alkaline conditions, and later resolved as described in FIGS. 12a-c. Lane 1—excised gel band from lane 3 of the gel presented in FIG. 16a; lane 2—excised gel band from lane 4 of the gel presented in FIG. 16a. Note the presence of a ˜32 kDa band (in lane 2 of FIG. 16c) representing BCL-X_{L} and the presence of a ˜33 kDa band (in lane 2 of FIG. 16d) representing Mtch2 demonstrating that tBID forms a complex with either BCL-X_{L} or Mtch2. The asterisk marks cross-reactive substances that might have been released from the gel sections.

FIG. 17 is a schematic presentation of the two apoptotic pathways triggered by tBID to reach complete Cyt c release. The first pathway, which is BH3-dependent (BH3-dep), involves the oligomerization of BAX and BAK (BAX/BAK-dep) in the outer mitochondrial membrane (OMM), enabling the release of the initial 15% of Cyt c stored in the inter membrane space (IMS). A second pathway, which is BH3-independent (BH3-indep) but dependent on the PTP(PTP-dep), involves inner mitochondrial membrane (IMM) remodeling that results in the release of the remaining 85% of Cyt c stored in the cristae.

FIGS. 18a-b are Mtch2 (FIG. 18a) and tubulin (FIG. 18b) Western blot analyses of downregulated HeLa cells. HeLa cells transfected with the empty pRETRO-SUPER vector (cont.) or the pRETRO-SUPER 370 vector (Mtch2 DR) were cultured for three days in the presence of 1 μg/ml puromycin. On the fourth day, the cells were seeded and grown until single clones appeared. The clones were grown up and part of the cells from each of the clones was lysed, and equal amounts of protein (20 μg per lane) were subjected to SDS-PAGE and immunoblotting. Lane 1-cont., lane 2—Mtch2 DR. Note the decreased intensity of the 33 kDa band representing Mtch2 in Mtch2 DR HeLa cells. The asterisk indicates a nonspecific band.

FIG. 18c-a graph depicting the effect of Mtch2 downregulation on cell death. HeLa cells transfected with the pRETRO-SUPER 370 vector (Mtch2 DR, clones 1 and 2) or the empty vector (cont. clone 1), or parental, untransfected, HeLa cells were treated with Fas ligand (100 nM together with 1 μg/ml CHX) and at the indicated time points the cells were collected and cell death was monitored by FACScan using propidium iodide (PI) dye exclusion. The data represent the means±SEM of pooled results from three independent experiments. Note the sharp increase in percent of cell death of Mtch2 DR HeLa cells as compared to either control or parental HeLa cells, demonstrating that downregulating the expression of Mtch2 accelerates Fas-induced apoptosis.

FIGS. 19a-b are Western blot analyses of Mtch2 DR HeLa cells using anti HA (detecting tBID; FIG. 19a) or anti-cytochrome c (FIG. 19b) antibodies. FIG. 19a-Mitochondria prepared from HeLa cells transfected with an empty pRETRO-SUPER vector (cont) or pRETRO-SUPER 370 (Mtch2 DR) were incubated with a 293T cytosol containing HA-tBID. At the end of the reaction mitochondria were separated from the soluble fraction by centrifugation, treated with cross-linker, lysed and size-fractionated by SDS-PAGE. Lane 1—HeLa cells transfected with the control vector; lane 2—HeLa cells transfected with the Mtch2 DR vector which forms a short double stranded RNA in the form of stem and loop (shRNA). Note the significant reduction in the 45 kDa tBID-Mtch2 complex in Mtch2 DR HeLa cells (lane 2) as compared to control HeLa cells (lane 1). FIG. 19b-Mitochondria prepared from HeLa cells transfected with an empty pRETRO-SUPER vector (cont.; lanes 1, 3, 5) or pRETRO-SUPER 370 (Mtch2 DR; lanes 2, 4, 6) were incubated for 1 minute (1′; lanes 1 and 2), 5 minutes (5′; lanes 3 and 4) or 10 minutes (10′; lanes 5 and 6) with 80 pmol of recombinant tBID. Mitochondria were then separated from the soluble fraction by centrifugation and the soluble fraction was analyzed by Western blot using anti-cytochrome c Abs. Note the increase in Cyt c level in HeLa cells transfected with the pRETRO-SUPER 370 in which Mtch2 is downregulated by shRNA along with extended incubation with tBID, demonstrating that downregulating Mtch2 results in accelerated tBID-induced cytochrome c release.

FIG. 20 is a Western blot analysis depicting Mtch2 expression in various mice tissues. Seven-week old wild-type C57BL/6 female mice were sacrificed by cervical dislocation and the indicated organs were removed and immediately frozen in liquid nitrogen. The organs were then thawed, homogenized, and equal amounts of protein (50 μg from each organ) were subjected to SDS-PAGE, followed by Western blot analysis using anti-Mtch2 Abs. Lane—brain, lane 2—heart, lane 3-lung, lane 4—thymus, lane 5—spleen, lane 6—liver, lane 7—pancreas, lane 8-intestine, lane 9—kidney, lane 10—ovary. The asterisk marks a crossreactive band. Note the high expression of Mtch2 in the heart and the liver and the moderate expression level in the brain, intestine and kidney.

FIGS. 21a-c depict the generation of Mtch2 knock out mice by gene targeting. FIG. 21a—A diagram of the MTCH2 genomic locus, the targeting vector and the homologous recombinant between the wild-type (WT) locus and the knockout (KO) locus. The gene scheme depicts the exons (empty boxes 1-13). The targeting vector includes the following domains: TK (Thymidine kinase), SH (short homology arm), neo (neomycine gene), and LH (long homology arm). Also indicated are the restriction enzyme sites (NcoI and SpeI) and the position of external 3′ and 5′ probes. FIGS. 21b-c are Southern blot analyses of DNA from R1 ES clones using the 5′ (FIG. 21b) or the 3′ (FIG. 21c) probes. SpeI digest was hybridized with 5′-probe and represents the homologues recombination of the LH (FIG. 21b), and NcoI digest was hybridized with 3′-probe and represents the homologues recombination of the SH (FIG. 21c), demonstrating a recombinant R1 ES clone. Note the presence of the two bands (13 and 8.8 kb) using the SpeI digest (lane 2, FIG. 21b) and the two bands (6.4 and 4.6 kb) using the NcoI digest (lane 2, FIG. 21c) demonstrating the presence of a heterozygout mouse (+/−) resulting of homologous recombination of the Mtch2 targeting vector. +/+represents wild-type animal; +/−represents heterozygout animal.

FIG. 22 is a schematic illustration depicting a possible model for interplay between BCL-2 family members and Mtch2 at the mitochondria. Mtch2 resides in the OMM in a relatively large complex that might include IMM proteins. In response to TNFα, tBID and BAX are recruited to the Mtch2-resident complex. At the mitochondria, tBID induces the oligomerization of BAX, which results in the release of Cyt c and caspase activation. In cells overexpressing BCL-X_{L}, the TNFα-induced recruitment of tBID and BAX to the Mtch2-resident complex is partially inhibited, along with the inhibition of Cyt c release.

### DESCRIPTION OF THE PREFERRED EMBODIMENTS

The present invention is of methods of regulating apoptosis in a cell using an agent capable of modulating the expression of an MCD-containing protein (e.g., Mtch2) capable of tBID binding activity and/or the tBID binding activity of the MCD-containing protein. Specifically, the present invention can be used to treat pathologies associated with disregulated apoptosis such as cancer of neurodegenerative diseases.

The principles and operation of a method of regulating apoptosis according to the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Apoptosis, or programmed cell death, is a naturally occurring form of cell suicide playing a crucial role in ensuring the normal development and maintenance of cells, organs, and tissues. Excessive apoptosis is associated with pathological cell loss associated with degenerative disorders such as Alzheimer's disease, Parkinson's disease, amyotrophic lateral sclerosis (ALS) and retinitis pigmentosa, hematological disorders and viral infections including human immunodeficiency virus (HIV)-induced acquired immunodeficiency syndrome (AIDS). Stimuli which can trigger excessive apoptosis include growth factors such as tumor necrosis factor (TNF), Fas, and transforming growth factor (TGF)-β. Alternatively, insufficient or failure of apoptosis, which may be triggered by various factors such as growth factors, extracellular matrix changes, CD40 ligand and viral gene products is a major contributor to the pathogenesis of cancer, autoimmune disorders, bacterial infections, and viral infections.

Apoptosis is regulated by various proteins such as the members of BCL-2 family. One of the pro-apoptotic BCL-2 family members is the BID protein, which includes the amphipathic α-helical BH3 domain. During apoptosis, cleavage of cytosolic BID by active caspase-8 results in a truncated BID fragment (tBID) that translocates to the mitochondria and induces the activation of BAX and BAK resulting in the release of Cyt c and caspase activation. Most importantly, BID is required for apoptosis in-vivo since Bid-deficient mice are resistant to Fas and TNFα-induced hepatocellular apoptosis (Yin, X. M., et al., 1999, Nature, 400: 886-891; Zhao, Y., et al., 2001, J. Biol. Chem. 276: 27432-27440). Thus, the prior art data demonstrate that tBID is a critical mediator of apoptosis.

U.S. Pat. Appl. No. 20050250680 and Walensky L D, 2004 (Supra) describes the use of a BID-derived BH3 peptide, which mimics the apoptotic action of BID in regulation of apoptosis in leukemia cells.

Another approach for regulating apoptosis is based on the modulation of BID-interacting proteins such as BAK [Wei MC, 2000 (Supra)] and BAX [Eskes R, 2000 (Supra)] which are induced to homoligomerize by tBID during apoptosis; the mitochondrial permeability transition pore (mPTP) which was proposed to mediate tBID induced remodeling of mitochondrial structure thereby leading to redistribution of Cyt c stored in intra-mitochondrial cristae [Scorrano L, 2002 (Supra)]; and the BCL-2 and BCL-X_{L} molecules, which were suggested to interact with tBID [Cheng EH, 2001 (Supra)]. However, to date, modulation of the expression levels or activity of such molecules to optimally regulate apoptosis has not been demonstrated.

While reducing the present invention to practice, the present inventors have uncovered that Mtch2, a previously uncharacterized MCD-containing mitochondrial protein, binds to tBID and that modulation of the expression level and/or the tBID binding activity of such MCD-containing protein can be used to regulate apoptosis.

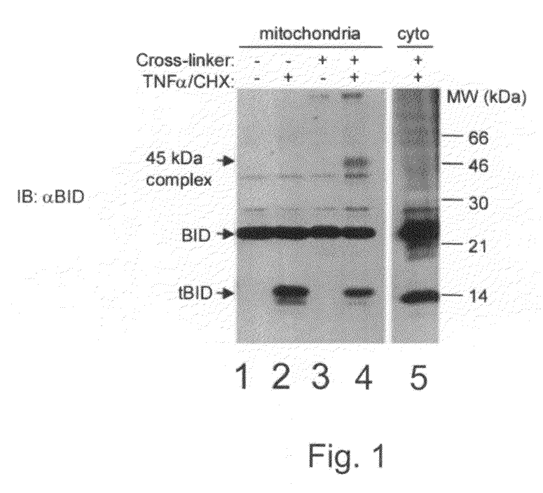

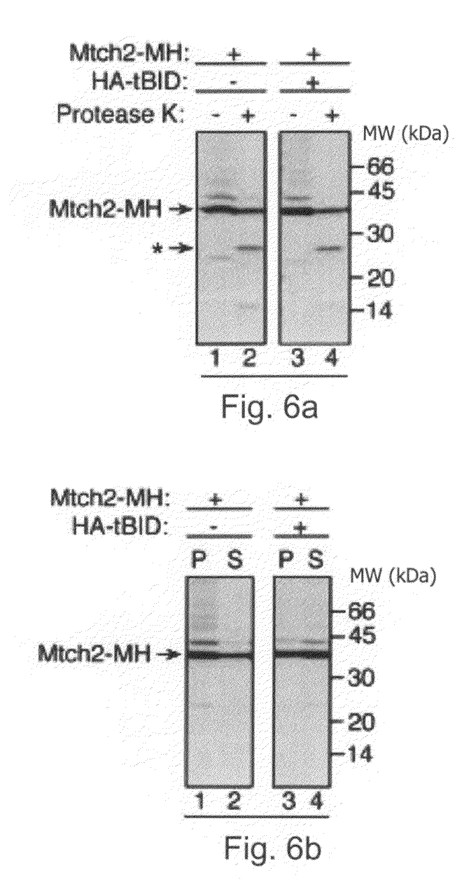

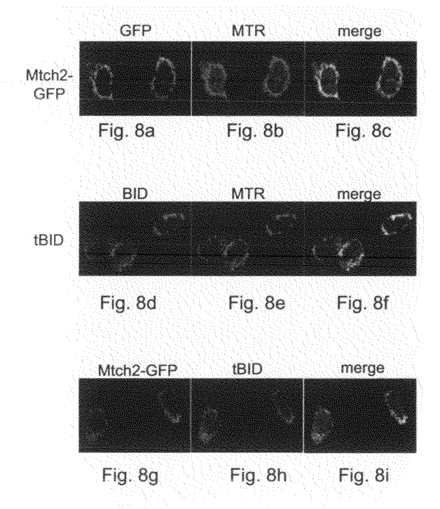

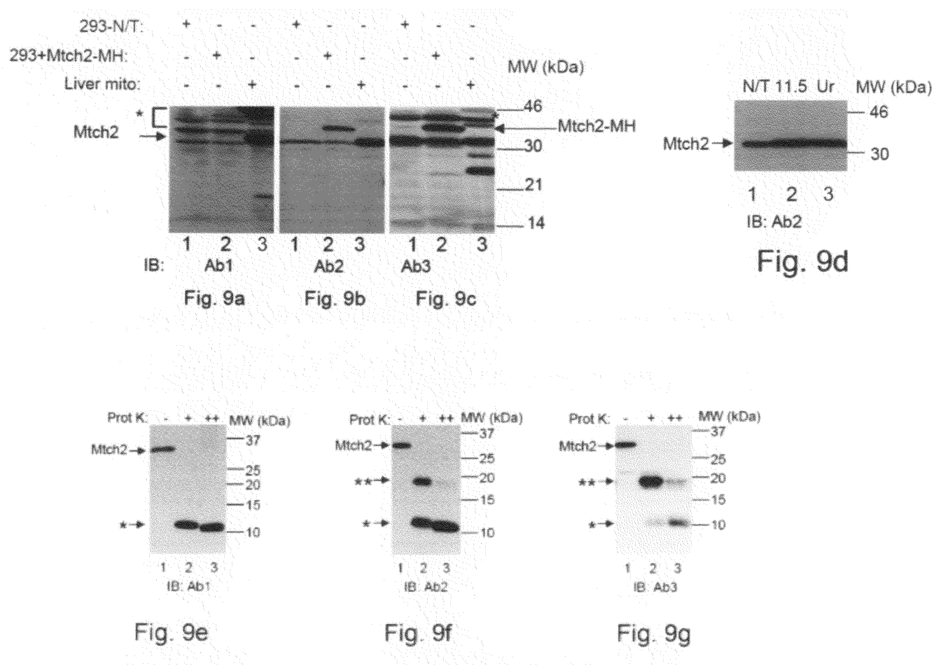

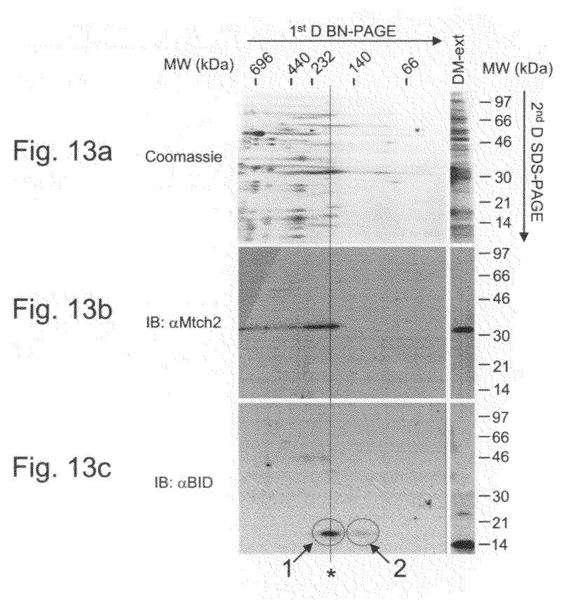

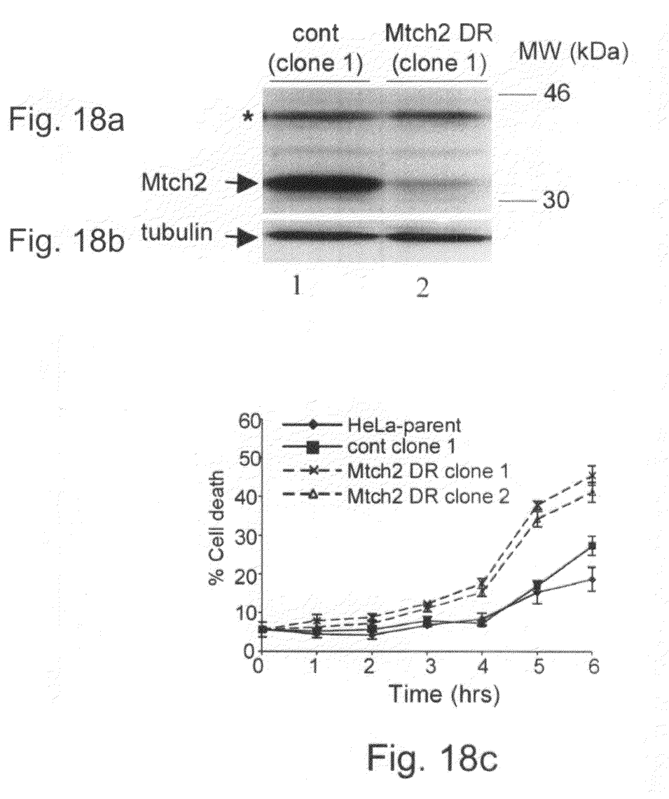

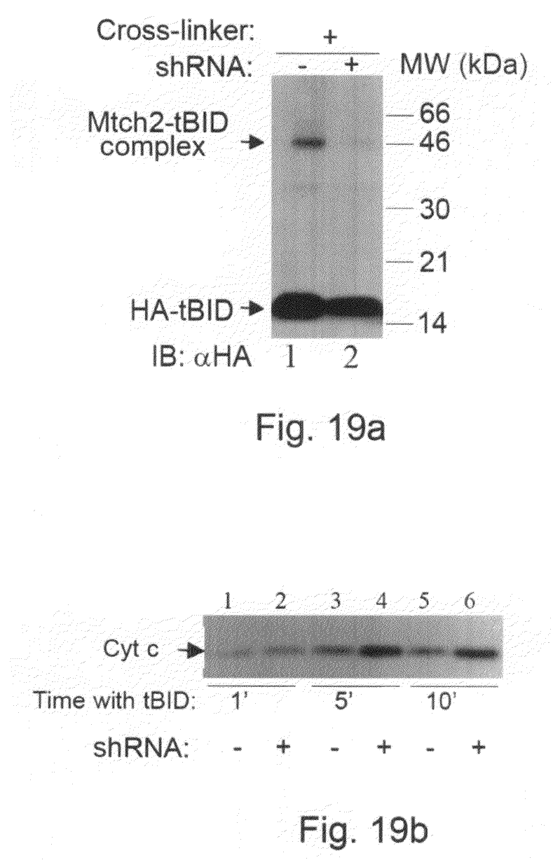

As is described in the Examples section which follows, Mtch2 was identified from a ˜45 kDa tBID-immunoreactive cross-linkable complex (FIGS. 1-3, Example 1). In addition, using recombinant, affinity-tagged tBID and Mtch2 proteins the present inventors demonstrated that such a complex can be formed in vitro (FIGS. 5a-c, Example 2). Moreover, confocal microscopy analysis revealed that Mtch2 and tBID co-localize to the mitochondria of HeLa cells (FIGS. 8a-i, Example 5) and that the presence of tBID at the mitochondria destabilizes the membrane-attachment of Mtch2 (FIGS. 6a-b, Example 3). The present inventors further generated anti-Mtch2 polyclonal antibodies (Ab1, Ab2 and Ab3) and characterized the tBID-interacting domain on the Mtch2 protein (FIGS. 9-11, Example 6). In addition, induction of apoptosis using TNFα resulted in the recruitment of both tBID and BAX to the ˜185 kDa Mtch2-mitochondrial resident complex (FIGS. 13-15, Example 8) and, on the other hand, overexpression of BCL-X_{L} resulted in increased formation of a tBID-BCL-X_{L} complex and thus partially inhibited the recruitment of tBID to the Mtch2-resident complex (FIGS. 15a-g and 16a-d, Example 9). Moreover, downregulation of Mtch2 in HeLa cells resulted in increased rates of Fas-induced apoptosis and Cyt c release (FIGS. 18-19, Example 10).

Thus, according to one aspect of the present invention there is provided a method of regulating apoptosis in a cell. The method is effected by contacting the cell with an agent capable of modulating: (i) an expression of an MCD-containing protein capable of tBID binding activity; and/or (ii) the tBID binding activity of the MCD-containing protein.

As used herein the phrase “regulating apoptosis” refers to upregulating or downregulating the rate and/or level of apoptosis, a programmed cell death machinery whereby the cell executes a “cell suicide” program. Apoptosis plays a crucial role in ensuring the normal development and maintenance of cells, organs, and tissues and involves in a number of physiological events such as embryogenesis, regulation of the immune system, and homeostasis. Thus, apoptosis can be in response to diverse signals such as stimulation by growth factors (e.g., TNFα and Fas), limb and neural development, neurodegenerative diseases, radiotherapy and chemotherapy as well as environmental conditions. Apoptotic processes are usually characterized by uncoupling of mitochondrial oxidation, decreased levels of nicotinamide adenine dinucleotide phosphate [NAD(P)H], release of cytochrome c, activation of caspases, DNA fragmentation and externalization of phosphatidylserine (a membrane phospholipid normally restricted to the inner leaflet of the lipid bilayer) to the outer leaflet of the plasma membrane (described in length in the preceding background section).

Non-limiting examples of cells of the present invention include, fetal or adult blood cells, bone marrow cells, neuronal cells, cardiac cells and the like.

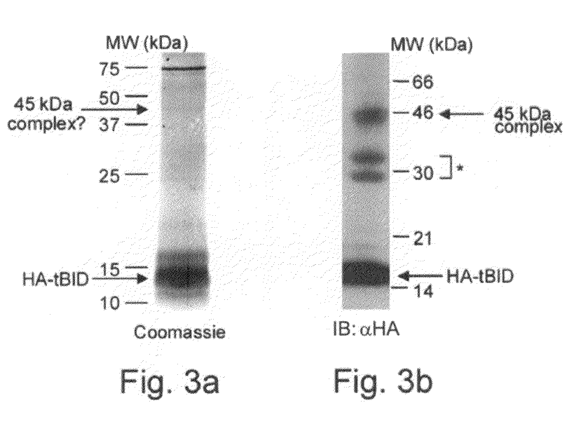

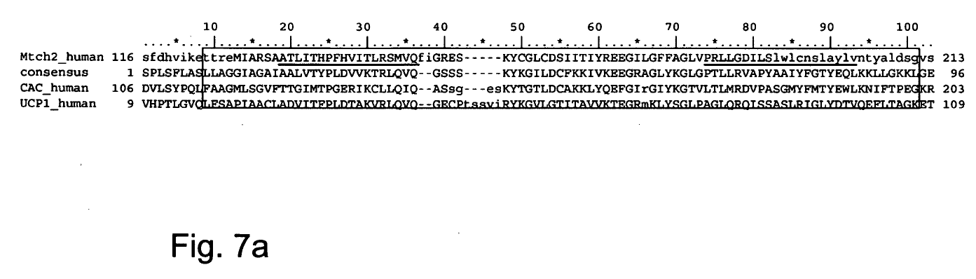

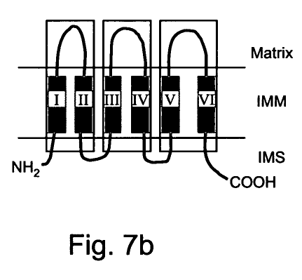

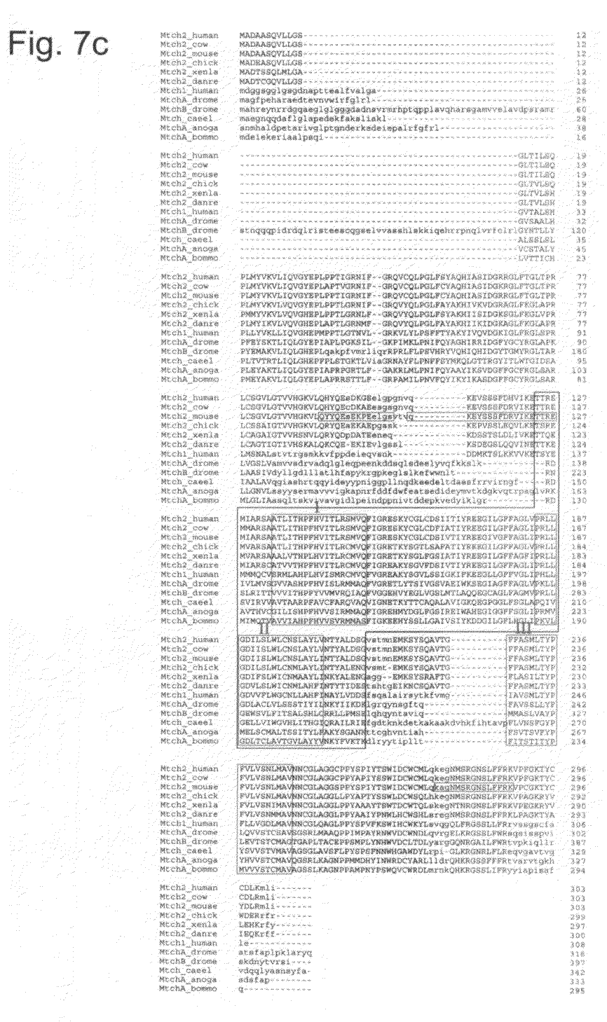

As used herein the phrase “MCD-containing protein” refers to a protein which includes at least one of the mitochondrial carrier domain (MCD) that is found in various members of the mitochondrial carrier protein (MCP) family. The MCD is about 100 amino acids and comprises two hydrophobic stretches that are of sufficient length to span the membrane as α-helices, separated by an extensive hydrophilic region (Walker, J. E. and Runswick, M. J. 1993, J. Bioenerg. Biomembr. 25: 435-446). For example, the MCD of human Mtch2 corresponds to amino acids 128-192 of the polypeptide set forth by SEQ ID NO:7 (GenBank Accession No. NP_{—}055157). Non-limiting examples of MCD-containing proteins which can be used according to this aspect of the present invention include, human Mtch2 (GenBank Accession No. NP_{—}055157; SEQ ID NO:7), human mitochondrial carnitine/acylcarnitine translocase (CAC) (GenBank Accession No. 043772; SEQ ID NO:9), human mitochondrial uncoupling protein 1 (UCP1) (GenBank Accession No. P25874; SEQ ID NO:13), bovine Mtch2 (GenBank Accession No. BAA95942; SEQ ID NO:15), mouse Mtch2 (GenBank Accession No. AAD52647; SEQ ID NO:17), chicken Mtch2 (GenBank Accession No. BAA86900; SEQ ID NO:14), Xenopus laevis (xenla) Mtch2 (GenBank Accession No. BAB03397; SEQ ID NO:18), zebrafish Mtch2 (GenBank Accession No. NP_{—}571457; SEQ ID NO:16), human Mtch1 (GenBank Accession No. AAF12793; SEQ ID NO:6), Drosophila melanogaster MtchA (GenBank Accession No. AAD52649; SEQ ID NO:22), Drosophila melanogaster MtchB (GenBank Accession No. AAM48309; SEQ ID NO:23), Caenorhabditis elegans Mtch (GenBank Accession No. NP_{—}495545; SEQ ID NO:19), Anopheles gambiae str. PEST MtchA (GenBank Accession No. EAA04685; SEQ ID NO:20) and Bombyx mori (bommo) MtchA (GenBank Accession No. BAA86901; SEQ ID NO:21). Multiple alignments of the MCD-containing proteins are shown in FIGS. 7a, c and d and in FIG. 3c in Grinberg et al., 2005 (Supra).

The term “tBID” refers to the C-terminal truncated fragment of the BH3 interacting death agonist (BID) protein which results from the enzymatic cleavage of cytosolic BID (e.g., by active caspase). As is mentioned in the background section, at an early stage of apoptosis, tBID translocates to the mitochondria and mediates the release of Cyt c therefrom. Non-limiting examples of tBID proteins include the mouse tBID (amino acids 60-195 of SEQ ID NO:8; GenBank Accession No. AAC71064) and human tBID (amino acids 61-195 of SEQ ID NO:24; GenBank Accession No. CAG30275).

As is illustrated in the Examples section which follows, the MCD-containing protein of the present invention is capable of tBID binding activity.

As used herein the phrase “tBID binding activity” refers to the ability of the MCD-containing protein of the present invention to bind tBID and form a stable complex therewith. Such a binding activity can be detected using various approaches known in the art, such as using a cross-linker agent (e.g., sulfo-BSOCOES, Pierce Biotechnology, Rockford, Ill.) followed by Western blot analysis as described in the Examples section which follows, a yeast-two-hybrid system or co-immunoprecipitation.

According to a preferred embodiment of this aspect of the present invention, regulating apoptosis is upregulating apoptosis, i.e., increasing the rate of apoptosis.

Preferably, in order to upregulate apoptosis, the agent used by the present invention is capable of decreasing (i.e., downregulating) the expression level of the MCD-containing protein of the present invention.

Decreasing the expression level of the MCD-containing protein can be effected on the genomic and/or the transcript level using a variety of molecules which interfere with transcription and/or translation (e.g., antisense, siRNA, Ribozyme, DNAzyme), or on the protein level using e.g., antagonists, enzymes that cleave the polypeptide and the like.

Following is a list of agents capable of decreasing the expression level MCD-containing protein of the present invention.

One example of an agent capable of downregulating (or decreasing the expression level of) the MCD-containing protein of the present invention is an antibody or antibody fragment capable of specifically binding to the MCD-containing protein. Preferably, the antibody specifically binds at least one epitope of the MCD-containing protein (e.g., Mtch2). As used herein, the term “epitope” refers to any antigenic determinant on an antigen to which the paratope of an antibody binds.

Epitopic determinants usually consist of chemically active surface groupings of molecules such as amino acids or carbohydrate side chains and usually have specific three dimensional structural characteristics, as well as specific charge characteristics.

For example, to downregulate the expression level of human Mtch2, the antibody can be directed against an epitope (e.g., a peptide of 3-8 amino acids) selected from the polypeptide set forth by SEQ ID NO:7. For example, as is shown in Example 6 of the Examples section which follows, the present inventors generated antibodies directed against the peptides set forth by SEQ ID NOs:29, 30 and 31 which were selected from the mouse Mtch2 polypeptide (SEQ ID NO:17).

The term “antibody” as used in this invention includes intact molecules as well as functional fragments thereof, such as Fab, F(ab′)2, Fv or single domain molecules such as VH and VL to an epitope of an antigen. These functional antibody fragments are defined as follows: (1) Fab, the fragment which contains a monovalent antigen-binding fragment of an antibody molecule, can be produced by digestion of whole antibody with the enzyme papain to yield an intact light chain and a portion of one heavy chain; (2) Fab′, the fragment of an antibody molecule that can be obtained by treating whole antibody with pepsin, followed by reduction, to yield an intact light chain and a portion of the heavy chain; two Fab′ fragments are obtained per antibody molecule; (3) (Fab′)₂, the fragment of the antibody that can be obtained by treating whole antibody with the enzyme pepsin without subsequent reduction; F(ab′)2 is a dimer of two Fab′ fragments held together by two disulfide bonds; (4) Fv, defined as a genetically engineered fragment containing the variable region of the light chain and the variable region of the heavy chain expressed as two chains; (5) Single chain antibody (“SCA”), a genetically engineered molecule containing the variable region of the light chain and the variable region of the heavy chain, linked by a suitable polypeptide linker as a genetically fused single chain molecule; and (6) Single domain antibodies are composed of a single VH or VL domains which exhibit sufficient affinity to the antigen.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference and the Examples section which follows).

Antibody fragments according to the present invention can be prepared by proteolytic hydrolysis of the antibody or by expression in E. coli or mammalian cells (e.g. Chinese hamster ovary cell culture or other protein expression systems) of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R. [Biochem. J. 73: 119-126 (1959)]. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, as described in Inbar et al. [Proc. Nat'l Acad. Sci. USA 69:2659-62 (19720]. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by Whitlow and Filpula, Methods 2: 97-105 (1991); Bird et al., Science 242:423-426 (1988); Pack et al., Bio/Technology 11:1271-77 (1993); and U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry [Methods, 2: 106-10 (1991)].

Humanized forms of non-human (e.g., murine) antibodies are chimeric molecules of immunoglobulins, immunoglobulin chains or fragments thereof (such as Fv, Fab, Fab′, F(ab′).sub.2 or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introduction of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10,: 779-783 (1992); Lonberg et al., Nature 368: 856-859 (1994); Morrison, Nature 368 812-13 (1994); Fishwild et al., Nature Biotechnology 14, 845-51 (1996); Neuberger, Nature Biotechnology 14: 826 (1996); and Lonberg and Huszar, Intern. Rev. Immunol. 13, 65-93 (1995).

It will be appreciated that targeting of particular compartment within the cell can be achieved using intracellular antibodies (also known as “intrabodies”). These are essentially SCA to which intracellular localization signals have been added (e.g., ER, mitochondrial, nuclear, cytoplasmic). This technology has been successfully applied in the art (for review, see Richardson and Marasco, 1995, TIBTECH vol. 13). Intrabodies have been shown to virtually eliminate the expression of otherwise abundant cell surface receptors and to inhibit a protein function within a cell (See, for example, Richardson et al., 1995, Proc. Natl. Acad. Sci. USA 92: 3137-3141; Deshane et al., 1994, Gene Ther. 1: 332-337; Marasco et al., 1998 Human Gene Ther 9: 1627-42; Shaheen et al., 1996 J. Virol. 70: 3392-400; Werge, T. M. et al., 1990, FEBS Letters 274:193-198; Carlson, J. R. 1993 Proc. Natl. Acad. Sci. USA 90:7427-7428; Biocca, S. et al., 1994, Bio/Technology 12: 396-399; Chen, S-Y. et al., 1994, Human Gene Therapy 5:595-601; Duan, L et al., 1994, Proc. Natl. Acad. Sci. USA 91:5075-5079; Chen, S-Y. et al., 1994, Proc. Natl. Acad. Sci. USA 91:5932-5936; Beerli, R. R. et al., 1994, J. Biol. Chem. 269:23931-23936; Mhashilkar, A. M. et al., 1995, EMBO J. 14:1542-1551; PCT Publication No. WO 94/02610 by Marasco et al.; and PCT Publication No. WO 95/03832 by Duan et al.).

To prepare an intracellular antibody expression vector, the cDNA encoding the antibody light and heavy chains specific for the target protein of interest are isolated, typically from a hybridoma that secretes a monoclonal antibody specific for the marker. Hybridomas secreting anti-marker monoclonal antibodies, or recombinant monoclonal antibodies, can be prepared using methods known in the art. Once a monoclonal antibody specific for the marker protein is identified (e.g., either a hybridoma-derived monoclonal antibody or a recombinant antibody from a combinatorial library), DNAs encoding the light and heavy chains of the monoclonal antibody are isolated by standard molecular biology techniques. For hybridoma derived antibodies, light and heavy chain cDNAs can be obtained, for example, by PCR amplification or cDNA library screening. For recombinant antibodies, such as from a phage display library, cDNA encoding the light and heavy chains can be recovered from the display package (e.g., phage) isolated during the library screening process and the nucleotide sequences of antibody light and heavy chain genes are determined. For example, many such sequences are disclosed in Kabat, E. A., et al. (1991) Sequences of Proteins of Immunological Interest, Fifth Edition, U.S. Department of Health and Human Services, NIH Publication No. 91-3242 and in the “Vbase” human germline sequence database. Once obtained, the antibody light and heavy chain sequences are cloned into a recombinant expression vector using standard methods.

For mitochondrial expression of the light and heavy chains, the nucleotide sequences encoding the mitochondrial targeting sequences are added [e.g., the COOH-terminal signal anchor of Bcl-2 (Nguyen, M. et al., 1993. J. Biol. Chem. 268:25265-25268)]. An intracellular antibody expression vector can encode an intracellular antibody in one of several different forms. For example, in one embodiment, the vector encodes full-length antibody light and heavy chains such that a full-length antibody is expressed intracellularly. In another embodiment, the vector encodes a full-length light chain but only the VH/CH1 region of the heavy chain such that a Fab fragment is expressed intracellularly. In another embodiment, the vector encodes a single chain antibody (scFv) wherein the variable regions of the light and heavy chains are linked by a flexible peptide linker and expressed as a single chain molecule. To inhibit marker activity in a cell, the expression vector encoding the intracellular antibody is introduced into the cell by standard transfection methods, as discussed hereinbefore.

Another agent capable of downregulating the MCD-containing protein of the present invention is a small interfering RNA (siRNA) molecule. RNA interference is a two step process. In the first step, which is termed as the initiation step, input dsRNA is digested into 21-23 nucleotide (nt) small interfering RNAs (siRNA), probably by the action of Dicer, a member of the RNase III family of dsRNA-specific ribonucleases, which processes (cleaves) dsRNA (introduced directly or via a transgene or a virus) in an ATP-dependent manner. Successive cleavage events degrade the RNA to 19-21 bp duplexes (siRNA), each with 2-nucleotide 3′ overhangs [Hutvagner and Zamore Curr. Opin. Genetics and Development 12:225-232 (2002); and Bernstein Nature 409:363-366 (2001)].

In the effector step, the siRNA duplexes bind to a nuclease complex to from the RNA-induced silencing complex (RISC). An ATP-dependent unwinding of the siRNA duplex is required for activation of the RISC. The active RISC then targets the homologous transcript by base pairing interactions and cleaves the mRNA into 12 nucleotide fragments from the 3′ terminus of the siRNA [Hutvagner and Zamore Curr. Opin. Genetics and Development 12:225-232 (2002); Hammond et al. (2001) Nat. Rev. Gen. 2:110-119 (2001); and Sharp Genes. Dev. 15:485-90 (2001)]. Although the mechanism of cleavage is still to be elucidated, research indicates that each RISC contains a single siRNA and an RNase [Hutvagner and Zamore Curr. Opin. Genetics and Development 12:225-232 (2002)].

Because of the remarkable potency of RNAi, an amplification step within the RNAi pathway has been suggested. Amplification could occur by copying of the input dsRNAs which would generate more siRNAs, or by replication of the siRNAs formed. Alternatively or additionally, amplification could be effected by multiple turnover events of the RISC [Hammond et al. Nat. Rev. Gen. 2:110-119 (2001), Sharp Genes. Dev. 15:485-90 (2001); Hutvagner and Zamore Curr. Opin. Genetics and Development 12:225-232 (2002)]. For more information on RNAi see the following reviews Tuschl ChemBiochem. 2:239-245 (2001); Cullen Nat. Immunol. 3:597-599 (2002); and Brantl Biochem. Biophys. Act. 1575:15-25 (2002).

Synthesis of RNAi molecules suitable for use with the present invention can be effected as follows. First, the mRNA sequence encoding the MCD-containing protein (e.g., Mtch2; SEQ ID NO:28; GenBank Accession No. NM_{—}014342) is scanned downstream of the AUG start codon for AA dinucleotide sequences. Occurrence of each AA and the 3′ adjacent 19 nucleotides is recorded as potential siRNA target sites. Preferably, siRNA target sites are selected from the open reading frame, as untranslated regions (UTRs) are richer in regulatory protein binding sites. UTR-binding proteins and/or translation initiation complexes may interfere with binding of the siRNA endonuclease complex [Tuschl ChemBiochem. 2:239-245]. It will be appreciated though, that siRNAs directed at untranslated regions may also be effective, as demonstrated for GAPDH wherein siRNA directed at the 5′ UTR mediated about 90% decrease in cellular GAPDH mRNA and completely abolished protein level (www.ambion.com/techlib/tn/91/912.html).

Second, potential target sites are compared to an appropriate genomic database (e.g., human, mouse, rat etc.) using any sequence alignment software, such as the BLAST software available from the NCBI server (www.ncbi.nlm.nih.gov/BLAST/). Putative target sites which exhibit significant homology to other coding sequences are filtered out.

Qualifying target sequences are selected as template for siRNA synthesis. Preferred sequences are those including low G/C content as these have proven to be more effective in mediating gene silencing as compared to those with G/C content higher than 55%. Several target sites are preferably selected along the length of the target gene for evaluation. For better evaluation of the selected siRNAs, a negative control is preferably used in conjunction. Negative control siRNA preferably include the same nucleotide composition as the siRNAs but lack significant homology to the genome. Thus, a scrambled nucleotide sequence of the siRNA is preferably used, provided it does not display any significant homology to any other gene.

The selected siRNAs can be chemically synthesized oligonucleotides (using e.g., solid phase synthesis) or can be encoded from plasmids in order to induce RNAi in cells following transfection (using e.g., the pRETRO-SUPER vector as described in the Examples section which follows). Recently, retrovirus- or lentivirus-delivered RNAi were developed and were found efficient in long-term gene silencing in vivo [Hao DL., et al., 2005, Acta. Biochim. Biophys. Sin. (Shanghai), 37(11): 779-83].

For example, a suitable Mtch2 siRNA can be the siRNA set forth by SEQ ID NO:27 which was found efficient in downregulating the expression level of Mtch2 in HeLa cells (see Example 10 of the Examples section which follows).

Another agent capable of downregulating the MCD-containing protein of the present invention is a DNAzyme molecule capable of specifically cleaving an mRNA transcript or DNA sequence of the MCD-containing protein. DNAzymes are single-stranded polynucleotides which are capable of cleaving both single and double stranded target sequences (Breaker, R. R. and Joyce, G. Chemistry and Biology 1995; 2:655; Santoro, S. W. & Joyce, G. F. Proc. Natl, Acad. Sci. USA 1997; 943:4262) A general model (the “10-23” model) for the DNAzyme has been proposed. “10-23” DNAzymes have a catalytic domain of 15 deoxyribonucleotides, flanked by two substrate-recognition domains of seven to nine deoxyribonucleotides each. This type of DNAzyme can effectively cleave its substrate RNA at purine:pyrimidine junctions (Santoro, S. W. & Joyce, G. F. Proc. Natl, Acad. Sci. USA 199; for rev of DNAzymes see Khachigian, L M [Curr Opin Mol Ther 4:119-21 (2002)].

Examples of construction and amplification of synthetic, engineered DNAzymes recognizing single and double-stranded target cleavage sites have been disclosed in U.S. Pat. No. 6,326,174 to Joyce et al. DNAzymes of similar design directed against the human Urokinase receptor were recently observed to inhibit Urokinase receptor expression, and successfully inhibit colon cancer cell metastasis in vivo (Itoh et al., 20002, Abstract 409, Ann Meeting Am Soc Gen Ther www.asgt.org). In another application, DNAzymes complementary to bcr-ab1 oncogenes were successful in inhibiting the oncogenes expression in leukemia cells, and lessening relapse rates in autologous bone marrow transplant in cases of CML and ALL.

Downregulation of the MCD-containing protein of the present invention can also be effected by using an antisense polynucleotide capable of specifically hybridizing with an mRNA transcript encoding the MCD-containing protein (e.g., Mtch2).

Design of antisense molecules which can be used to efficiently down-regulate the MCD-containing protein must be effected while considering two aspects important to the antisense approach. The first aspect is delivery of the oligonucleotide into the cytoplasm of the appropriate cells, while the second aspect is design of an oligonucleotide which specifically binds the designated mRNA within cells in a way which inhibits translation thereof.

The prior art teaches of a number of delivery strategies which can be used to efficiently deliver oligonucleotides into a wide variety of cell types [see, for example, Luft J Mol Med 76: 75-6 (1998); Kronenwett et al. Blood 91: 852-62 (1998); Rajur et al. Bioconjug Chem 8: 935-40 (1997); Lavigne et al. Biochem Biophys Res Commun 237: 566-71 (1997) and Aoki et al. (1997) Biochem Biophys Res Commun 231: 540-5 (1997)].

In addition, algorithms for identifying those sequences with the highest predicted binding affinity for their target mRNA based on a thermodynamic cycle that accounts for the energetics of structural alterations in both the target mRNA and the oligonucleotide are also available [see, for example, Walton et al. Biotechnol Bioeng 65: 1-9 (1999)].

Such algorithms have been successfully used to implement an antisense approach in cells. For example, the algorithm developed by Walton et al. enabled scientists to successfully design antisense oligonucleotides for rabbit beta-globin (RBG) and mouse tumor necrosis factor-alpha (TNF alpha) transcripts. The same research group has more recently reported that the antisense activity of rationally selected oligonucleotides against three model target mRNAs (human lactate dehydrogenase A and B and rat gp130) in cell culture as evaluated by a kinetic PCR technique proved effective in almost all cases, including tests against three different targets in two cell types with phosphodiester and phosphorothioate oligonucleotide chemistries.

In addition, several approaches for designing and predicting efficiency of specific oligonucleotides using an in vitro system were also published (Matveeva et al., Nature Biotechnology 16: 1374-1375 (1998)].

For example, a suitable antisense oligonucleotides targeted against the mRNA coding for the MCD-containing protein of the present invention (e.g., Mtch2) would be of the following sequences: 5′-TCCTCACCCTTGTCACTCTCC-3′ [SEQ ID NO:25; corresponds to nucleic acids 314-334 of SEQ ID NO:28; designed using the IDT design tool (http://www.idtdna.com) according to the Matveeva rule set].

Several clinical trials have demonstrated safety, feasibility and activity of antisense oligonucleotides. For example, antisense oligonucleotides suitable for the treatment of cancer have been successfully used [Holmund et al., Curr Opin Mol Ther 1:372-85 (1999)], while treatment of hematological malignancies via antisense oligonucleotides targeting c-myb gene, p53 and Bcl-2 had entered clinical trials and had been shown to be tolerated by patients [Gerwitz Curr Opin Mol Ther 1:297-306 (I 999)].

More recently, antisense-mediated suppression of human heparanase gene expression has been reported to inhibit pleural dissemination of human cancer cells in a mouse model [Uno et al., Cancer Res 61:7855-60 (2001)].

Thus, the current consensus is that recent developments in the field of antisense technology which, as described above, have led to the generation of highly accurate antisense design algorithms and a wide variety of oligonucleotide delivery systems, enable an ordinarily skilled artisan to design and implement antisense approaches suitable for downregulating expression of known sequences without having to resort to undue trial and error experimentation.

Another agent capable of downregulating the expression of the MCD-containing protein is a ribozyme molecule capable of specifically cleaving an mRNA transcript encoding the MCD-containing protein (e.g., Mtch2, SEQ ID NO:28). Ribozymes are being increasingly used for the sequence-specific inhibition of gene expression by the cleavage of mRNAs encoding proteins of interest [Welch et al., Curr Opin Biotechnol. 9:486-96 (1998)]. The possibility of designing ribozymes to cleave any specific target RNA has rendered them valuable tools in both basic research and therapeutic applications. In the therapeutics area, ribozymes have been exploited to target viral RNAs in infectious diseases, dominant oncogenes in cancers and specific somatic mutations in genetic disorders [Welch et al., Clin Diagn Virol. 10:163-71 (1998)]. Most notably, several ribozyme gene therapy protocols for HIV patients are already in Phase 1 trials. More recently, ribozymes have been used for transgenic animal research, gene target validation and pathway elucidation. Several ribozymes are in various stages of clinical trials. ANGIOZYME was the first chemically synthesized ribozyme to be studied in human clinical trials. ANGIOZYME specifically inhibits formation of the VEGF-r (Vascular Endothelial Growth Factor receptor), a key component in the angiogenesis pathway. Ribozyme Pharmaceuticals, Inc., as well as other firms have demonstrated the importance of anti-angiogenesis therapeutics in animal models. HEPTAZYME, a ribozyme designed to selectively destroy Hepatitis C Virus (HCV) RNA, was found effective in decreasing Hepatitis C viral RNA in cell culture assays (Ribozyme Pharmaceuticals, Incorporated—WEB home page).

Another agent which can be used to downregulate the expression level of the MCD-containing protein of the present invention in cells is a triplex forming oligonucleotide (TFO). Recent studies have shown that TFOs can be designed which can recognize and bind to polypurine/polypirimidine regions in double-stranded helical DNA in a sequence-specific manner. These recognition rules are outlined by Maher III, L. J., et al., Science, 1989, 245:725-730; Moser, H. E., et al., Science, 1987, 238:645-630; Beal, P. A., et al, Science, 1992, 251:1360-1363; Cooney, M., et al., Science, 1988, 241:456-459; and Hogan, M. E., et al., EP Publication 375408. Modification of the oligonucleotides, such as the introduction of intercalators and backbone substitutions, and optimization of binding conditions (pH and cation concentration) have aided in overcoming inherent obstacles to TFO activity such as charge repulsion and instability, and it was recently shown that synthetic oligonucleotides can be targeted to specific sequences (for a recent review see Seidman and Glazer, J Clin Invest 2003; 112:487-94).

In general, the triplex-forming oligonucleotide has the sequence correspondence:

| | | | | |
|---|---|---|---|---|
| oligo | 3′--A | G | G | T |
| duplex | 5′--A | G | C | T |
| duplex | 3′--T | C | G | A |

However, it has been shown that the A-AT and G-GC triplets have the greatest triple helical stability (Reither and Jeltsch, BMC Biochem, 2002, 3: 27.). The same authors have demonstrated that TFOs designed according to the A-AT and G-GC rule do not form non-specific triplexes, indicating that the triplex formation is indeed sequence specific.

Thus for any given sequence in the MCD-containing protein regulatory region (e.g., the tBID-binding domain included in amino acids 205-303 of SEQ ID NO:7) a triplex forming sequence may be devised. Triplex-forming oligonucleotides preferably are at least 15, more preferably 25, still more preferably 30 or more nucleotides in length, up to 50 or 100 bp.

Transfection of cells (for example, via cationic liposomes) with TFOs, and formation of the triple helical structure with the target DNA induces steric and functional changes, blocking transcription initiation and elongation, allowing the introduction of desired sequence changes in the endogenous DNA and resulting in the specific downregulation of gene expression. Examples of such suppression of gene expression in cells treated with TFOs include knockout of episomal supFG1 and endogenous HPRT genes in mammalian cells (Vasquez et al., Nucl Acids Res. 1999; 27:1176-81, and Puri, et al, J Biol Chem, 2001; 276:28991-98), and the sequence- and target specific downregulation of expression of the Ets2 transcription factor, important in prostate cancer etiology (Carbone, et al, Nucl Acid Res. 2003; 31:833-43), and the pro-inflammatory ICAM-1 gene (Besch et al, J Biol Chem, 2002; 277:32473-79). In addition, Vuyisich and Beal have recently shown that sequence specific TFOs can bind to dsRNA, inhibiting activity of dsRNA-dependent enzymes such as RNA-dependent kinases (Vuyisich and Beal, Nuc. Acids Res 2000; 28:2369-74).

Additionally, TFOs designed according to the abovementioned principles can induce directed mutagenesis capable of effecting DNA repair, thus providing both downregulation and upregulation of expression of endogenous genes (Seidman and Glazer, J Clin Invest 2003; 112:487-94). Detailed description of the design, synthesis and administration of effective TFOs can be found in U.S. Patent Application Nos. 2003 017068 and 2003 0096980 to Froehler et al, and 2002 0128218 and 2002 0123476 to Emanuele et al, and U.S. Pat. No. 5,721,138 to Lawn.

According to another preferred embodiment of this aspect of the present invention, upregulation of apoptosis is achieved using an agent capable of stabilizing the MCD-containing protein-tBID complex. It will be appreciated that such agents can be for example a peptide, an antibody (e.g., a stabilizing antibody) or a small molecule which, when added to cells expressing the MCD-containing protein-tBID complex, is capable of stabilizing (i.e., prolonging the half-life) of such a complex.

The term “peptide” as used herein encompasses synthetic or naturally occurring peptides, peptide analogues or mimetics thereof. The term “peptide” preferably refers to short amino acid sequences of at least 2 or 3, preferably at least 4, more preferably, at least 5, more preferably, in the range of 5-30, even more preferably in the range of 5-25 natural or non-natural amino acids which are capable of the biological activity (i.e., stabilizing the MCD-containing protein-tBID complex in this case). Further description of natural and non-natural amino acids is provided in PCT Appl. No. IL2004/000744, which is fully incorporated herein by reference.

As used herein the term “mimetics” refers to molecular structures, which serve as substitutes for the peptide of the present invention in performing the biological activity (Morgan et al. (1989) Ann. Reports Med. Chem. 24:243-252 for a review of peptide mimetics). Peptide mimetics, as used herein, include synthetic structures (known and yet unknown), which may or may not contain amino acids and/or peptide bonds, but retain the structural and functional features of the peptide. Types of amino acids which can be utilized to generate mimetics are further described hereinbelow. The term, “peptide mimetics” also includes peptoids and oligopeptoids, which are peptides or oligomers of N-substituted amino acids [Simon et al. (1972) Proc. Natl. Acad. Sci. USA 89:9367-9371]. Further included as peptide mimetics are peptide libraries, which are collections of peptides designed to be of a given amino acid length and representing all conceivable sequences of amino acids corresponding thereto. Methods of producing peptide mimetics are described hereinbelow.

The peptides of present invention can be biochemically synthesized such as by using standard solid phase techniques. These methods include exclusive solid phase synthesis, partial solid phase synthesis methods, fragment condensation and classical solution synthesis. These methods are preferably used when the peptide is relatively short (i.e., 10 kDa) and/or when it cannot be produced by recombinant techniques (i.e., not encoded by a nucleic acid sequence) and therefore involve different chemistry.

Solid phase peptide synthesis procedures are well known in the art and further described by John Morrow Stewart and Janis Dillaha Young, Solid Phase Peptide Syntheses (2nd Ed., Pierce Chemical Company, 1984).

Synthetic peptides can be purified by preparative high performance liquid chromatography [Creighton T. (1983) Proteins, structures and molecular principles. WH Freeman and Co. N.Y.] and the composition of which can be confirmed via amino acid sequencing.

In cases where large amounts of the peptides of the present invention are desired, the peptides of the present invention can be generated using recombinant techniques such as described by Bitter et al., (1987) Methods in Enzymol. 153:516-544, Studier et al. (1990) Methods in Enzymol. 185:60-89, Brisson et al. (1984) Nature 310:511-514, Takamatsu et al. (1987) EMBO J. 6:307-311, Coruzzi et al. (1984) EMBO J. 3:1671-1680, Brogli et al., (1984) Science 224:838-843, Gurley et al. (1986) Mol. Cell. Biol. 6:559-565 and Weissbach & Weissbach, 1988, Methods for Plant Molecular Biology, Academic Press, NY, Section VIII, pp 421-463.

Combinatorial chemical, antibody or peptide libraries may be used to screen a plurality of agents.

It will be appreciated that when utilized along with automated equipment, the above-described method can be used to screen multiple agents both rapidly and easily.

According to other preferred embodiments of the present invention, regulating apoptosis is downregulating apoptosis.

Preferably, the agent which can be used to downregulate apoptosis is capable of increasing (i.e., upregulating) the expression level of the MCD-containing protein.

Upregulation of the expression level of the MCD-containing protein of the present invention can be effected at the genomic level (i.e., activation of transcription via promoters, enhancers, regulatory elements), at the transcript level (i.e., correct splicing, polyadenylation, activation of translation) or at the protein level (i.e., post-translational modifications).

Preferably, upregulation of the expression level of the MCD-containing protein of the present invention is effected by contacting the cell with an exogenous polypeptide including at least a functional portion of the MCD-containing protein.

As used herein, the phrase “polypeptide” encompasses a naturally occurring polypeptide which is comprised solely of natural amino acid residues or synthetically prepared polypeptides, comprised of a mixture of natural and modified (non-natural) amino acid residues as described hereinabove.

The phrase “functional portion” as used herein refers to at least a portion of the polypeptide of the present invention which is sufficient to down-regulate apoptosis.

It will be appreciated that for large polypeptides (e.g., above 25 amino acids), the exogenous polypeptide is preferably prepared using recombinant techniques.

For example, to generate the MCD-containing protein of the present invention (a recombinant polypeptide such as human Mtch2; SEQ ID NO:7), a polynucleotide sequence encoding the MCD-containing protein (e.g., GenBank Accession number NM_{—}014342; SEQ ID NO:28) or a functional portion thereof is preferably ligated into a nucleic acid construct suitable for expression in a host cell. Such a nucleic acid construct includes a promoter sequence for directing transcription of the polynucleotide sequence in the cell in a constitutive or inducible manner.

Constitutive promoters suitable for use with the present invention are promoter sequences which are active under most environmental conditions and most types of cells such as the cytomegalovirus (CMV) and Rous sarcoma virus (RSV). Inducible promoters suitable for use with the present invention include for example the tetracycline-inducible promoter [Zabala M, et al., Cancer Res. 2004, 64(8): 2799-804].

The nucleic acid construct (also referred to herein as an “expression vector”) of the present invention includes additional sequences which render this vector suitable for replication and integration in prokaryotes, eukaryotes, or preferably both (e.g., shuttle vectors). In addition, a typical expression vector may also contain a transcription and translation initiation sequence, enhancers (e.g., SV40 early gene enhancer; see also Enhancers and Eukaryotic Expression, Cold Spring Harbor Press, Cold Spring Harbor, N.Y. 1983), transcription and translation terminator, and a polyadenylation signal which may increase the efficiency of mRNA translation (e.g., the GU or U rich sequences located downstream from the polyadenylation site and a highly conserved sequence of six nucleotides, AAUAAA, located 11-30 nucleotides upstream). It will be appreciated that in order to secret the recombinant polypeptide from the host cell (i.e., a cell in which the polynucleotide of the present invention is expressed) the expression vector of the present invention typically includes a signal sequence for secretion.

The expression vector of the present invention can further include additional polynucleotide sequences that allow, for example, the translation of several proteins from a single mRNA such as an internal ribosome entry site (IRES) and sequences for genomic integration of the promoter-chimeric polypeptide.

Other than containing the necessary elements for the transcription and translation of the inserted coding sequence, the expression construct of the present invention can also include sequences engineered to enhance stability, production, purification, yield or toxicity of the expressed polypeptide.

As mentioned hereinabove, a variety of cells can be used as host-expression systems to express the recombinant polypeptide of the present invention (e.g., human Mtch2). These include, but are not limited to, microorganisms, such as bacteria transformed with a recombinant bacteriophage DNA, plasmid DNA or cosmid DNA expression vector containing the polypeptide coding sequence, mammalian expression systems, yeast transformed with recombinant yeast expression vectors containing the coding sequence (see for example, U.S. Pat. Application No: 5,932,447); plant cell systems infected with recombinant virus expression vectors (e.g., cauliflower mosaic virus, CaMV; tobacco mosaic virus, TMV) or transformed with recombinant plasmid expression vectors, such as Ti plasmid, containing the coding sequence [for suitable plant expression vectors see for example, Brisson et al. (1984) Nature 310:511-514; Takamatsu et al. (1987) EMBO J. 6:307-311; Coruzzi et al. (1984) EMBO J. 3:1671-1680; Brogli et al., (1984) Science 224:838-843; Gurley et al. (1986) Mol. Cell. Biol. 6:559-565; Weissbach & Weissbach, 1988, Methods for Plant Molecular Biology, Academic Press, NY, Section VIII, pp 421-463]. Bacterial systems are preferably used to produce recombinant polypeptides since they enable a high production volume at low cost.

In bacterial systems, a number of expression vectors can be advantageously selected depending upon the use intended for the polypeptide expressed. For example, when large quantities of polypeptide are desired, vectors that direct the expression of high levels of the protein product, possibly as a fusion with a hydrophobic signal sequence, which directs the expressed product into the periplasm of the bacteria or the culture medium where the protein product is readily purified may be desired. Certain fusion protein engineered with a specific cleavage site to aid in recovery of the polypeptide may also be desirable. Such vectors adaptable to such manipulation include, but are not limited to, the pET series of E. coli expression vectors [Studier et al., Methods in Enzymol. 185:60-89 (1990)].

Examples for mammalian expression vectors include, but are not limited to, pcDNA3, pcDNA3.1 (+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Stratagene, pTRES which is available from Clontech, and their derivatives.

Expression vectors containing regulatory elements from eukaryotic viruses such as retroviruses can be also used. SV40 vectors include pSVT7 and pMT2. Vectors derived from bovine papilloma virus include pBV-1MTHA, and vectors derived from Epstein Bar virus include pHEBO, and p205. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

Various methods can be used to introduce the expression vector of the present invention into host cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

Introduction of nucleic acids by viral infection offers several advantages over other methods such as lipofection and electroporation, since higher transfection efficiency and specificity can be obtained due to the infectious nature of viruses.

Transformed cells are cultured under effective conditions, which allow for the expression of high amounts of the recombinant polypeptide. Effective culture conditions include, but are not limited to, effective media, bioreactor, temperature, pH and oxygen conditions that permit protein production. An effective medium refers to any medium in which a cell is cultured to produce the recombinant polypeptide of the present invention. Such a medium typically includes an aqueous solution having assimilable carbon, nitrogen and phosphate sources, and appropriate salts, minerals, metals and other nutrients, such as vitamins. Cells of the present invention can be cultured in conventional fermentation bioreactors, shake flasks, test tubes, microtiter dishes and petri plates. Culturing can be carried out at a temperature, pH and oxygen content appropriate for a recombinant cell. Such culturing conditions are within the expertise of one of ordinary skill in the art.

Depending on the vector and host system used for production, resultant polypeptides of the present invention may either remain within the cell, secreted into the fermentation medium, secreted into a space between two cellular membranes, such as the periplasmic space in E. coli; or retained on the outer surface of a cell or viral membrane.

Following a predetermined time in culture, recovery of the recombinant polypeptide is effected. The phrase “recovery of the recombinant polypeptide” used herein refers to collecting the whole fermentation medium containing the polypeptide and need not imply additional steps of separation or purification.

Thus, polypeptides of the present invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential solubilization.

To facilitate recovery, the expressed coding sequence can be engineered to encode the polypeptide of the present invention and a fused cleavable moiety. Such a fusion protein can be designed so that the polypeptide can be readily isolated by affinity chromatography; e.g., by immobilization on a column specific for the cleavable moiety. Where a cleavage site is engineered between the polypeptide and the cleavable moiety, the polypeptide can be released from the chromatographic column by treatment with an appropriate enzyme or agent that specifically cleaves the fusion protein at this site [e.g., see Booth et al., Immunol. Lett. 19:65-70 (1988); and Gardella et al., J. Biol. Chem. 265:15854-15859 (1990)].

The polypeptide of the present invention is preferably retrieved in “substantially pure” form. As used herein, the phrase “substantially pure” refers to a purity that allows for the effective use of the recombinant polypeptide (i.e., the MCD-containing protein of the present invention) in downregulating apoptosis.

Another agent capable of upregulating the expression level of the MCD-containing protein of the present invention (e.g., Mtch2) is an exogenous polynucleotide sequence designed and constructed to express in cells at least a functional portion of the MCD-containing protein of the present invention. Accordingly, the exogenous polynucleotide sequence may be a DNA or RNA sequence encoding the MCD-containing protein of the present invention.

It will be appreciated that for ex vivo or in vivo gene therapy applications which are further described hereinunder the exogenous polynucleotide of the present invention is administered to the cell-of-interest (e.g., a cell in which apoptosis is upregulated) to thereby downregulate apoptosis. As used herein, the phrase “ex vivo gene therapy” refers to the process of expressing the polypeptide of the present invention in cell cultures derived from a subject (e.g., autologous or allogeneic cells) followed by administration of such cells (which express the recombinant polypeptide of the present invention) back into the subject in need of therapy. The phrase “in vivo gene therapy” refers to the process of expressing the polypeptide of the present invention in cells of the subject in need of therapy.

It will be appreciated that the type of viral vector and the specific promoter used for ex vivo or in vivo gene therapy will depend on the cell type transformed. The ability to select suitable vectors according to the cell type transformed is well within the capabilities of the ordinary skilled artisan and as such no general description of selection consideration is provided herein.

Recombinant viral vectors are useful for in vivo expression of recombinant proteins since they offer advantages such as lateral infection and targeting specificity. Lateral infection is inherent in the life cycle of, for example, retrovirus and is the process by which a single infected cell produces many progeny virions that bud off and infect neighboring cells. The result is that a large area becomes rapidly infected, most of which was not initially infected by the original viral particles. This is in contrast to vertical-type of infection in which the infectious agent spreads only through daughter progeny. Viral vectors can also be produced that are unable to spread laterally. This characteristic can be useful if the desired purpose is to introduce a specified gene into only a localized number of targeted cells.

Currently preferred in vivo nucleic acid transfer techniques include transfection with viral or non-viral constructs, such as adenovirus, lentivirus, Herpes simplex I virus, or adeno-associated virus (AAV) and lipid-based systems. Useful lipids for lipid-mediated transfer of the gene are, for example, DOTMA, DOPE, and DC-Chol [Tonkinson et al., Cancer Investigation, 14(1): 54-65 (1996)]. The most preferred constructs for use in gene therapy are viruses, most preferably adenoviruses, AAV, lentiviruses, or retroviruses. A viral construct such as a retroviral construct includes at least one transcriptional promoter/enhancer or locus-defining element(s), or other elements that control gene expression by other means such as alternate splicing, nuclear RNA export, or post-translational modification of messenger. Such vector constructs also include a packaging signal, long terminal repeats (LTRs) or portions thereof, and positive and negative strand primer binding sites appropriate to the virus used, unless it is already present in the viral construct. By way of example, such constructs will typically include a 5′ LTR, a tRNA binding site, a packaging signal, an origin of second-strand DNA synthesis, and a 3′ LTR or a portion thereof. Other vectors can be used that are non-viral, such as cationic lipids, polylysine, and dendrimers.

Downregulation of apoptosis can be also achieved using an agent capable of decreasing (i.e., downregulating) the tBID binding activity of the MCD-containing protein of the present invention. Such an agent can be, for example, an antibody, a peptide or a small molecule capable of preventing the formation or destabilizing the MCD-containing protein-tBID complex.

It will be appreciated that such an antibody which is used to decrease the tBID-binding activity of the MCD-containing protein or to destabilize the complex therebetween can be directed against the MCD-containing protein (e.g., the Ab1, Ab2 or the Ab3 anti-Mtch2 antibodies generated by the present inventors and which are described in Example 6 of the Examples section which follows), and preferably, against the tBID-binding domain on the MCD-containing protein of the present invention. A non-limiting example of such an epitope can be any of the peptides set forth by SEQ ID NOs:32-111 which were selected from the tBID-binding domain on Mtch2 (amino acids 205-303 of SEQ ID NO:7). Additionally or alternatively, the antibody which is used to destabilize the MCD-containing protein-tBID complex can be directed against the complex itself. It will be appreciated that such antibodies can be generated using methods known in the art by injecting a substantially pure preparation of the MCD-containing protein-tBID complex into an immunizing animal.

The peptide used to decrease the tBID-binding activity of the MCD-containing protein or to destabilize the MCD-containing protein-tBID complex can be any peptide (from e.g., 5 amino acids to 25 amino acids in length) having the affinity to both proteins at the site of interaction therebetween. According to presently preferred configurations, such a peptide is derived from the C-terminal region of Mtch2 (i.e., from amino acids 205-303 of SEQ ID NO:7) which was found by the present inventors to be the tBID-binding domain on Mtch2 (see Example 6 of the Examples section which follows). Non-limiting examples of such peptides are set forth by SEQ ID NOs:32-111.

It will be appreciated that various methods can be used to qualify the ability of the agents of the present invention to upregulate or downregulate apoptosis as needed. These include detecting the expression level of the RNA encoding the MCD-containing protein (using e.g., Northern Blot analysis, RT-PCR analysis, RNA in situ hybridization stain, In situ RT-PCR stain) or the MCD-containing protein itself (using e.g., Western blot, Enzyme linked immunosorbent assay (ELISA), Radio-immunoassay (RIA), Fluorescence activated cell sorting (FACS), Immunohistochemical analysis) following contacting the cells with the agent of the present invention.

Following is a non-limiting description of a method of qualifying the agents capable of modulating the tBID-binding activity of the MCD-containing protein.

In order to qualify agents for the ability to increase or decrease the tBID binding activity of the MCD-containing protein or to stabilize or destabilize the MCD-containing protein-tBID complex, the complex can be formed in vitro by transfecting cells with expression vectors harboring the coding sequences of the MCD-containing protein and the tBID, essentially as described in Example 2 of the Examples section which follows. Briefly, cells are transfected with expression vectors encoding recombinant MCD-containing protein (e.g., Mtch2) and recombinant tBID. Confirmation of complex formation can be achieved by subjecting a protein extract derived from the transfected cells to Western blot analysis using antibodies specific to each component of the complex (i.e., an anti-MCD-containing protein and an anti-tBID antibody). Alternatively, the candidate agent can be administered to the cells prior to or along with the induction of expression of the recombinant proteins. Still alternatively, the agents can be contacted with the MCD-containing protein or tBID. The MCD-containing protein or tBID proteins are preferably bound to a solid support to monitor binding of the agent to the MCD-containing protein or the tBID proteins or to monitor stabilization (e.g., in the presence of alkali pH) of the pre-established complex, respectively. The solid support may be any material known to those of ordinary skill in the art to which a specific antibody which can recognize the MCD-containing protein or the tBID proteins may be attached, such as a test well in a microtiter plate, a nitrocellulose filter or another suitable membrane. Alternatively, the support may be a bead or disc, such as glass, fiberglass, latex or a plastic such as polystyrene or polyvinylchloride. Molecular immobilization on a solid support is effected using a variety of techniques known to those in the art.

Qualified agents which are capable of modulating the expression level or the tBID-binding activity of the MCD-containing protein can be further qualified by functional assays, such as by monitoring the effect of the agent on apoptosis in cells. The level of apoptosis in cells and tissues can be determined using various methods such as the Ethidium homodimer-1 staining (Invitrogen-Molecular Probes), the Tunnel assay (Roche, Basel, Switzerland), the Live/dead viability/cytotoxicity two-color fluorescence assay (Molecular Probes, Inc., L-3224, Eugene, Oreg., USA), FACS analysis [using molecules capable of specifically binding cells undergoing apoptosis, such as propidium iodide (see FIG. 18c) and Annexin V], and those of skills in the art are capable of assessing such levels in order to determine the standards of normal levels.

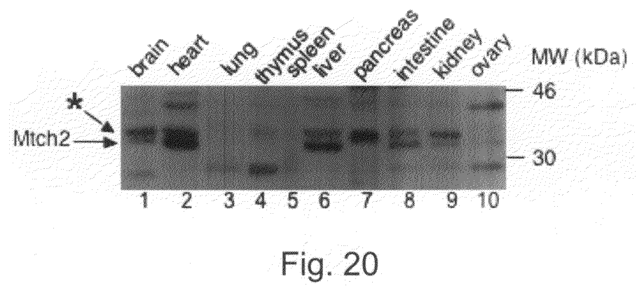

For example, as is shown in FIGS. 18a-c and is described Example 10 of the Examples section which follows the present inventors showed that the Mtch2 siRNA agent (SEQ ID NO:27) is capable of efficiently decreasing the expression level of Mtch2 and upregulating apoptosis in HeLa cells. In addition, as is shown in FIG. 20 and is described in Example 11 of the Examples section which follows, Mtch2 is highly expressed in the heart and liver and moderately expressed in the brain, intestine and kidney. Thus, the effect of the agents of the present invention in regulating apoptosis can be determined in the relevant tissues or cell lines derived therefrom (e.g., liver cancer cell lines, neuroblastoma or glioblastoma cells lines, and cardiac cancer). In addition, the effect of the agents of the present invention on apoptosis can be tested in vivo using the xenograft approach (Voskoglou-Nomikos T, et al., Clin. Cancer Res. 2003, 9: 4227-39) or in animal models for diseases such as Parkinson's, Alzheimer's and the like.

It will be appreciated that the agents described hereinabove which are capable of upregulating or downregulating apoptosis can be used to treat pathologies associated with disregulated apoptosis.

Thus, according to an additional aspect of the present invention there is provided a method of treating a pathology associated with disregulated apoptosis in a subject. The method is effected by administering to the subject the agent capable of modulating the expression of the MCD-containing protein capable of tBID binding activity; and/or the tBID binding activity of the MCD-containing protein.

The term “treating” refers to inhibiting, preventing, curing, reversing, attenuating, alleviating, minimizing, suppressing or halting the deleterious effects of a pathology and/or causing the reduction, remission, or regression of a pathology. Those of skill in the art will understand that various methodologies and assays can be used to assess the development of a pathology, and similarly, various methodologies and assays may be used to assess the reduction, remission or regression of the pathology.

The term “preventing” refers to keeping a pathology from occurring in a subject who may be at risk for having the pathology, but has not yet been diagnosed as having the pathology.

As used herein, the term “subject” (or “individual” which is interchangeably used herein) refers to an animal subject e.g., a mammal, e.g., a human being at any age who suffers from or is at risk of developing the pathology. Non-limiting examples of individuals who are at risk to develop the pathology of the present invention include individuals who are genetically predisposed to develop the pathology (e.g., individuals who carry a mutation or a DNA polymorphism which is associated with high prevalence of the pathology), and/or individuals who are at high risk to develop the pathology due to other factors such as environmental hazard or other pathologies.

As used herein the term “pathology” refers to any deviation from the normal structure and/or function of a particular cell, cell type, group of cells, tissue or organ leading to a disease, a disorder, a syndrome or an abnormal condition.

According to the method of this aspect of the present invention the pathology is associated with disregulated apoptosis. As used herein, the phrase “a pathology associated with disregulated apoptosis” relates to any pathology which is caused by or characterized by disregulated apoptosis. The phrase “disregulated apoptosis” relates to a rate and/or level of apoptosis which is above (i.e., abnormally high) or below (i.e., abnormally low) the level present in normal or unaffected cells of the same type or developmental stage.

Pathologies which are associated or characterized with abnormally high levels of apoptosis and which can be treated using the agents of the present invention include, but are not limited to, degenerative disorders such as neurological disorders [e.g., Alzheimer's disease, Parkinson's disease, amyotrophic lateral sclerosis (ALS) and retinitis pigmentosa], hematological disorders and viral infections including human immunodeficiency virus (HIV)-induced acquired immunodeficiency syndrome (AIDS).

Pathologies which are associated or characterized with abnormally low levels of apoptosis and which can be treated using the agents of the present invention include, but are not limited to, cancer, autoimmune disorders, bacterial infections, and viral infections.

Thus, in order to treat a pathology characterized by abnormally low level of apoptosis, the agent used by the method according to this aspect of the present invention is capable of upregulating apoptosis (e.g., by decreasing the expression level of the MCD-containing protein or by increasing the tBID binding activity of the MCD-containing protein). On the other hand, in order to treat a pathology characterized by abnormally high level of apoptosis, the agent used by the method according to this aspect of the present invention is capable of down-regulating apoptosis (e.g., by increasing the expression of the MCD-containing protein or by decreasing the tBID binding activity of the MCD-containing protein).

For example, in order to treat degenerated neuronal cells associated with abnormally high level of apoptosis (as in the case, for example, of Alzheimer's disease) an expression vector containing the Mtch2 polynucleotide (e.g., SEQ ID NO:28) can be targeted to the brain using liposomal and viral vectors as described in de Lima MC., et al., 2005, Curr Drug Targets CNS Neurol Disord. 4(4):453-65, which is fully incorporated herein by reference, and/or using a neuron-specific promoter such as the neurofilament promoter [Byrne et al. (1989) Proc. Natl. Acad. Sci. USA 86:5473-5477].

It will be appreciated that the agent of the present invention (e.g., the antibody, the siRNA, the exogenous polypeptide, the exogenous polynucleotide or the expression vector encoding same) can be administered to the subject per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the agent of the present invention (e.g., the antibody, the siRNA, the exogenous polypeptide, the exogenous polynucleotide or the expression vector encoding same) which is accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, neurosurgical strategies (e.g., intracerebral injection, intrastriatal infusion or intracerebroventricular infusion, intra spinal cord, epidural), transmucosal, intestinal or parenteral delivery, including intramuscular, subcutaneous and intramedullary injections as well as intrathecal, direct intraventricular, intracardiac, intravenous, intraperitoneal, intranasal, or intraocular injections.

Alternately, one may administer the pharmaceutical composition in a local rather than a systemic manner, for example, via injection of the pharmaceutical composition directly into a tissue region of a patient.

Intra-brain infusions can be performed using an implanted device as described in Sanftner L M, et al., 2005 (Exp Neurol. 194(2):476-83) for administration of recombinant adeno-associated virus (AAV2) to the brain.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichlorotetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose (i.e., a therapeutically effective amount as described hereinabove).

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide tissue levels of the active ingredient that are sufficient to regulate apoptosis (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions for human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

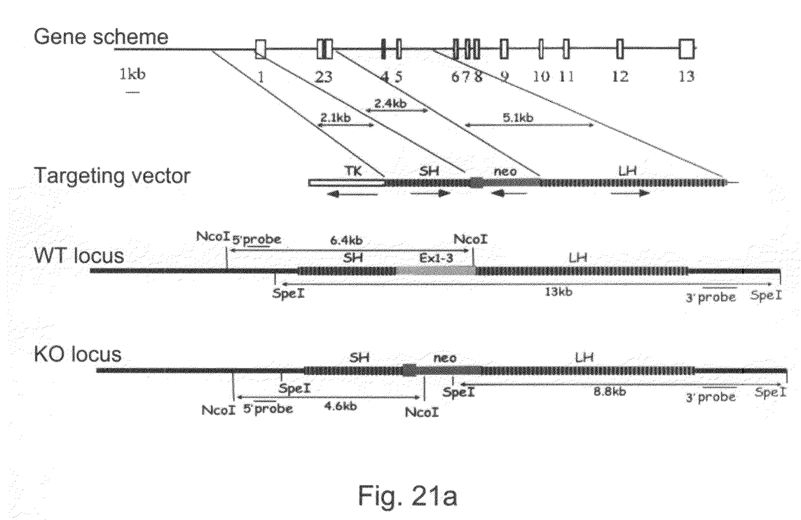

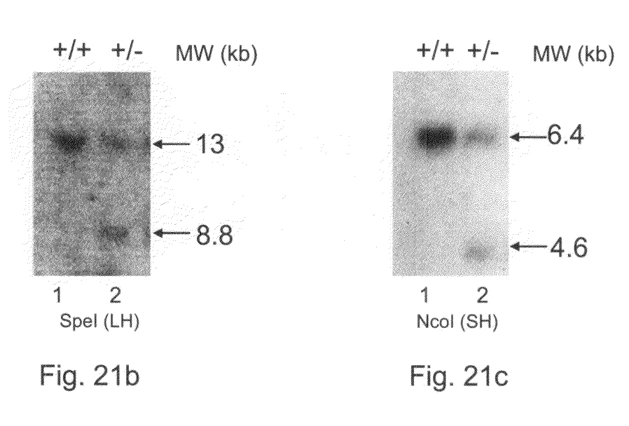

As is further shown in FIGS. 21a-c and is described in Example 11 of the Examples section which follows, the present inventors have generated genetically modified mice in which part of the gene encoding the Mtch2 is deleted (i.e., knock-out mice).

Thus, according to another aspect of the present invention, there is provided a multicellular organism comprising a genome which comprises a genetically modified Mtch2 gene being incapable of encoding a functional Mtch2 protein.

As used herein the phrase “multicellular organism” refers to any organism having more than one cell, preferably, with differentiated cells that perform specialized functions (e.g., neuronal system, digestive system, cardiovascular system and the like). Preferably, the phrase “multicellular organism” refers to mammals and their fetuses, but human, such as rodents (e.g., mouse, rat, guinea pig), monkeys (e.g., gorilla, chimpanzee, gibbon, rhesus, apes in particular), pigs, sheep, cattle etc. According to presently preferred embodiments of this aspect of the present invention, the multicellular organism is a mouse.

The phrase “genetically modified” refers to a sequence alteration which results in altered expression as compared with a wild type equivalent sequence. The sequence alteration may be natural or man-made. Preferably, it is a mutation in a structural part of a gene, however, control sequence (e.g., promoter, enhancer) mutations are not excluded. The alteration can be insertion, deletion and/or substitution of one or more nucleotides in one or more locations.

As used here the term “gene” refers to a nucleic acid sequence from which a protein can be expressed or a part thereof suitable, for example, for directing homologous recombination. Thus, a gene can include, for example, a complementary DNA sequence, a genomic DNA sequence or a mixed sequence of genomic and cDNA. Additional sequences can be included, such as, polylinkers, positive and negative selection sequences, and genetically modified sequences such as sequence alterations and the like. The introns of the gene, can be for example modified, e.g., shortened. However, the term gene as used herein further relates to the control sequences flanking the nucleic acid sequence from which a protein can be expressed, in particular upstream (5′) control sequences. Since a gene according to the present invention is expected to undergo homologous recombination, the term as used herein refers also to any portion of a gene that following the homologous recombination is capable of combining with endogenous sequences to reconstruct a nucleic acid sequence from which a protein can be expressed.

Preferably, the Mtch2 gene which is genetically modified in the multicellular organism of this aspect of the present invention is derived from mouse (nucleic acids 90697871-90717350 of GenBank Accession No. NC_{—}000068).

Following homologous recombination (using e.g., the Cre/LoxP system as described in U.S. Pat. No. 4,959,317 to Sauer, U.S. Pat. No. 6,924,146 to Wattler, et al. which are fully incorporated herein by reference), the genetically modified Mtch2 gene is incapable of encoding a functional Mtch2 protein. As used herein, the term “functional” refers to at least one function of the Mtch2 protein which involves binding tBID in the mitochondria and regulating apoptosis.

It will be appreciated that in cases the endogenous Mtch2 gene is necessary for survival in certain developmental stages (e.g., embryonic stages), the integration of the genetically modified Mtch2 gene in the genome of the targeted multicellular organism can be induced postnatally (i.e., after birth) or in the adult animal (e.g., in mice older than 1 month, 2 months, three months and the like).

For a delayed induction of homologous recombination (i.e., postnatally or in the adult animal), the targeting vector used for homologous recombination preferably includes additional elements such as Lox-P sites, which are used to delete the region/exons of interest. Such elements are generally described in Kuhn, R. and Schwenk, F., 2003. Methods Mol Biol 209:159-185.

Following homologous recombination, the modified Mtch2 gene comprises a deleted Mtch2 gene. For example, as is shown in FIGS. 21a-c the modified Mtch2 is lacking exons 1-3 of the 13 exons of the Mtch2 gene.

It will be appreciated that such genetically modified Mtch2 organism (e.g., mouse) can be used as an in vivo system to test the efficacy of the various agents of the present invention in regulating apoptosis. For example, an agent capable of upregulating the expression level of Mtch2 such as an exogenous Mtch2 polynucleotide (e.g., SEQ ID NO: 28) or an exogenous Mtch2 polypeptide (e.g., SEQ ID NO:7) can be administered to the Mtch2 knock-out mouse and the effect on apoptosis can be tested on biological samples (e.g., heart tissue, brain tissue, liver tissue) derived from the mouse using e.g., the Live/Dead viability/cytotoxicity two-color fluorescence assay (Molecular Probes, Inc., L-3224, Eugene, Oreg., USA) as described hereinabove. In addition, cells derived from Mtch2-knock-out (KO) mice can be used to test the specificity of agents that are targeting Mtch2 but not tBID and are designed to downregulate the tBID-binding activity of Mtch2. It will be appreciated that if such agents are specific to Mtch2 they are not expected to induce any effect on the Mtch2-KO mice.

As is mentioned before, in case the genetically modified gene is required for embryonic development and the homologous recombination is induced postnatally or in the adult animal, such an assay, which qualifies the efficacy of the agents of the present invention, can be performed also on the same animal or on parallel animal before and after the induction of homologous recombination.

As used herein the term “about” refers to +10%.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W.H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization-A Laboratory Course Manual” CSHL Press (1996); all of which are incorporated by reference as if fully set forth herein. Other general references are provided throughout this document. The procedures therein are believed to be well known in the art and are provided for the convenience of the reader.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

### General Materials and Experimental Methods

Cell lines—FL5.12, an IL-3-dependent murine early hematopoietic cell line, was maintained in ISCOVE's medium (Biological Industries, Beit Haemek, Israel) containing 10% fetal bovine serum supplemented with 10% WEHI-3B conditioned medium, as a source of IL-3. FL5.12-BCL-X_{L} cells, are FL5.12 cells that stably express mouse BCL-X_{L} (Gross A, 1999; J. Biol. Chem. 274:1156-1163). To induce apoptosis by means of TNFα, FL5.12 and FL5.12-BCL-X_{L} cells were treated for 5 hours with recombinant mouse TNFα (40 ng/ml; Sigma) and cycloheximide (CHX) (1 μg/ml; Sigma). 293T, an embryonic kidney cell line, 293T-T-Rex, a 293T stable clone expressing the tetracycline repressor (Invitrogene), and HeLa, a human cervical adenocarcinoma cell line, were maintained in DMEM containing 10% fetal bovine serum. Mouse embryonic fibroblasts (MEFs) were prepared from 11-13 day-old embryos, and maintained in ISCOVE's medium containing 10% fetal bovine serum. Bax, Bak double knockout MEFs were a generous gift from Stanley J. Korsmeyer (Dana-Farber Cancer Institute).

TBID expression plasmids—Wild-type p15 tBID was amplified by PCR from wild-type p22 BID (Wang, K., et al., 1996, Genes Dev 10:2859-6928). The pcDNA3 plasmid (Invitrogen Carlsbad Calif.) containing an HA epitope tag was ligated in-frame using the EcoRI-BamHI restriction sites to the tBID PCR product under the CMV promoter, to create the HA-tBID plasmid (pcDNA3-HA-tBID). To create the pT-Rex-HA-tBID inducible expression vector, the HA-tBID cDNA was PCR amplified from the pcDNA3-HA-tBID plasmid and the resulting PCR product was ligated using the EcoRI-BamHI restriction sites to the pCA14T-Rex vector, to create pT-Rex-HA-tBID.

Preparation of Mitochondria from Cultured Cells—Cells were Suspended in isotonic HIM buffer (200 mM mannitol, 70 mM sucrose, 1 mM EGTA, 10 mM HEPES, pH 7.5) and homogenized using either a polytron homogenizer (Brinkmann Instruments) at setting 6.5 for 10 seconds, or by passing them 20 times through a 25G (0.5×16) needle. Nuclei and unbroken cells were removed by centrifugation at 120 g for 5 minutes. The supernatant was centrifuged for 10 minutes at 10,000 g to collect the mitochondria-enriched fraction and the supernatant (cytosol).

Crude mitochondria were further purified according to Da Cruz et al. (Da Cruz, S., et al., 2003. J. Biol. Chem. 278: 41566-71). Briefly, 5-to-10 mg of crude mitochondria were suspended in 2.5 ml HIM buffer and loaded on a discontinuous nycodenz gradient. The gradient was prepared by stepwise layering of 3.5 ml of 22.5% nycodenz solution and 5.6 ml of 9.5% nycodenz solution in a 12 ml sw 45 Ti centrifuge tube. The nycodenz solutions themselves were prepared by diluting a 36% nycodenz solution with HIM buffer. The gradient was centrifuged (141,000 g at 4° C. for 1 hour) and the pellet obtained was resuspended in 10 ml HIM and centrifuged twice more (100,000 g at 4° C. for 10 minutes each) to obtain the final pellet.

Purification of the HA-tBID cross-linked complex—1000×10 cm plates of 293T cells were transiently transfected with pcDNA3-HA-tBID. Eighteen hours post-transfection, cells were harvested and subcellularly fractionated by differential centrifugation, as described above. The mitochondria-enriched heavy membrane fractions were treated with sulfo-Bis [2-(sulfosuccinimidooxy-carbonyloxy)ethyl]sulfone (sulfo-BSOCOES) at a final concentration of 10 mM. The cross-linking approach is preferable to co-immunoprecipitation from detergent-lysed cells or to yeast two-hybrid screening since tBID is an integral membrane protein and its interactions with membrane proteins are likely to depend on the presence of an intact membrane. After incubation for 30 minutes at room temperature, the cross-linker was quenched by the addition of 1 M Tris-HCl (pH 7.5) to a final concentration of 20 mM.

Following quenching, the membrane fraction was separated by centrifugation from the soluble fraction, and lysed in Laemli sample buffer (0.05 M TRIS pH 6.8, 3% SDS, 5% β-mercaptoethanol, 10% glycerol) without reducing agents. The resulting lysate was diluted in binding buffer [20 mM Tris (pH 7.5), 0.1 M NaCl, 0.1 M EDTA] to reach a final concentration of 0.2% SDS. The diluted lysate was incubated for 16 hours with 5 mg anti-HA mAbs coupled to agarose beads (Roche), followed by extensive washing of the beads with binding buffer containing 0.05% Tween 20. The material that remained bound to the beads was eluted by incubation for 15 minutes at 37° C. with 1 ml (1 mg/ml) HA peptide. Elution was repeated twice more, and the three eluates were pooled and concentrated, using a Centricon tube with a 3K cutoff (Amicon). The concentrated material was loaded onto a single lane, separated by SDS-PAGE, and then stained with Coomassie blue.

In gel proteolysis and mass spectrometry analysis—The stained protein bands or spots in the gel were cut with a clean razor blade. The proteins in the gel were then reduced with 10 mM DTT, and modified with 100 mM iodoacetamide in 10 mM ammonium bicarbonate. The gel pieces were treated with 50% acetonitrile in 10 mM ammonium bicarbonate to remove the stain from the proteins; the gel pieces were then dried. The dried gel pieces were rehydrated with 10% acetonitrile in 10 mM ammonium bicarbonate containing about 0.1 μg trypsin per sample. The gel pieces were then incubated overnight at 37° C. and the resulting peptides were recovered with 60% acetonitrile with 0.1% trifluoroacetate. The tryptic peptides were resolved by reverse-phase chromatography on 0.1×300-mm fused silica capillaries (100 micrometer ID, J&W) filled with porous R2 (Perspective). The peptides were then eluted using a 80-min linear gradient of 5 to 95% acetonitrile with 0.1% acetic acid in water, at a flow rate of 1 μl/minute. The liquid from the column was electrosprayed into an ion-trap mass spectrometer (LCQ, Finnegan, San Jose).

Mass spectrometry was performed in the positive ion mode, utilizing a repetitively full MS scan, followed by collision-induced dissociation (CID) of the most dominant ion selected from the first MS scan. The mass spectrometry data was compared to simulated proteolysis and CID of the proteins in the NR-NCBI database, using Sequest software (J. Eng, University of Washington, and J. Yates, Finnegan, San Jose). The amino terminal of the protein was sequenced on Peptide Sequencer 494A (Perkin Elmer) according to the manufacturer's instructions.

Cloning of the human Mtch2 gene and construction of Mtch2 expression plasmids—Single-stranded oligo-dT primed cDNA prepared from total RNA of HeLa cells was used as a template for PCR amplification of the human Mtch2 cDNA. The following primers were used for the PCR reaction: 5′-GGGAATTCATGGCGGACGCGGCCAG-3′ (sense primer; SEQ ID NO:1) and 5′-CCGGCTCCAATTAACATTTTCAGGTCAC-3′ (anti-sense primer; SEQ ID NO:2). The resulting PCR product was digested with EcoR1/BamH1, and subcloned into the EcoR1/BamH1 sites of pcDNA3.1/myc/His (Invitrogene). The sequence of human Mtch2 was confirmed by DNA sequencing. To create the Mtch2-GFP chimera, the human Mtch2 PCR product was ligated in-frame using the EcoRI-BamHI restriction sites to the N-terminus of EGFP in the pEGFP plasmid (Clontech).

Transient transfection system and adenoviral infections—Transient transfections were performed by the calcium phosphate method (Graham, F. L., and A. J. van der Eb., 1973, Virology 52: 456-67) or using lipofectamine 2000 (Gibco BRL) according to manufacturer's instructions. tBID adenoviral infection experiments were effected using an adenoviral vector with a constitutive expression of tBID produced as previously described (Sarig, R., Y. et al., 2003, J. Biol. Chem. 278: 10707-15). Viral preparations were made from freeze/thaw lysis of the cells, and viral titers were performed on 293T cells. For infection, cells were generally seeded at 70-80% confluence. Cells were infected with a multiplicity of infection (MOI) of 50 in a minimal culture medium volume for 1 hour; the medium volume was then increased until the cells were harvested.

Con-focal microscopy—To detect Mtch2-GFP and mitochondria, HeLa cells were transfected with Mtch2-GFP, and were incubated for 30 minutes at 37° C. with 100 nM mitotracker red (MTR; Molecular Probes) prior to fixation. To detect tBID and mitochondria, HeLa cells were transfected with tBID in the presence of the broad caspase inhibitor zVAD-fmk (50 μM; Biomol Research Laboratories), prestained with MTR, fixed, and immunostained with anti-BID Abs. To view Mtch2-GFP and tBID, HeLa cells were transfected with both Mtch2-GFP and tBID in the presence of zVAD-fink (50 μM), fixed, and immunostained with anti-BID Abs. The cover slips were mounted with elvanol, and the cells were viewed under a Nikon fluorescence microscope at a magnification of 400×. Pictures were taken with a 1310 digital camera (DVC). Confocal microscopy was performed using a Zeiss Axiovert 100 TV microscope (Oberkochen, Germany), attached to the Bio-Rad Radiance 2000 laser scanning system (Bio-Rad), operated by LaserSharp software.

Blue-native polyacrylamide gel electrophoresis (BN-PAGE)—BN-PAGE was performed according to Schagger (Schagger, H. 2001. Methods Cell Biol 65: 231-44). Briefly, purified mitochondria from FL5.12 cells were treated for 5 hours with TNFα/CHX or remained untreated, following which the mitochondria were solubilized with 0.3% n-Dodecyl β-D-maltoside (DM). Following high-speed centrifugation (217,000 g at 4° C. for 20 minutes) solubilized complexes were mixed with Coomassie blue G-250 prepared in 750 mM γ-caproic acid, so that the concentration of the dye was ¼ of the DM. The complexes were resolved on a 5-13% gradient native gel. In order to separate the complexes into their individual components, lanes from the native gels were incubated for 2 hours at room temperature while shaking with a buffer containing 50 mM Tris (pH 8.8), 6 M Urea, 30% Glycerol, 2% SDS, 1% DTT, and a trace of bromo phenol blue. The lanes were horizontally laid on an 8-20% gradient SDS-gel to separate the native complexes into their individual protein constitutes.

Mitochondrial import, alkali/urea extraction, and proteinase K treatment—In vitro mitochondrial import assays, urea extraction, and proteinase K treatment were performed as previously described (Goping, I. S., et al., 1998, J. Cell Biol. 143: 207-15). Briefly, mitochondria were isolated from mouse liver that was homogenized in HIM buffer, with two up and down strokes in a glass homogenizer. The homogenate was centrifuged for 10 minutes at 600 g. The supernatant was centrifuged for 15 minutes at 7,000 g. The pellet containing mitochondria was then resuspended in HIM and recentrifuged for 10 minutes at 600 g. The supernatant was centrifuged for 15 minutes at 7,000 g, and the mitochondrial pellet was resuspended in MRM-S (250 mM sucrose, 10 mM HEPES, 1 mM ATP, 5 mM succinate, 0.08 mM ADP, 2 mM K2HPO4 at pH 7.4).

For mitochondrial import experiments, mitochondria were incubated for 30 minutes at 30° C. with the cytosolic fraction of 293T cells (containing HA-tBID). At the end of the reaction, mitochondria were centrifuged, the pellet and supernatant fractions were solubilized in Laemli sample buffer (0.05 M TRIS pH 6.8, 3% SDS, 5% β-mercaptoethanol, 10% glycerol), and then analyzed by Western blot. For alkali extraction, the mitochondrial pellet was resuspended in 0.1 M Na₂CO₃ (pH 11.5). For urea extraction, the mitochondrial pellet was resuspended in 8 M urea. In both instances, mitochondria were incubated for 30 minutes at 4° C., followed by high-speed centrifugation (75,000 g) for 10 minutes. The resulting pellet and supernatant were solubilized in Laemli sample buffer, and analyzed by Western blot. For proteinase K treatment, the mitochondrial pellet was resuspended in SEM (250 mM sucrose, 10 mM MOPS/KOH, 2.5 mM EDTA) together with 0.1 or 1 μg/ml proteinase K, and incubated for 20 minutes at 4° C. The reaction was stopped with 1 mM PMSF and the mitochondria were centrifuged for 10 minutes at 10,000 g, resuspended in HIM containing 1 mM PMSF, and again recentrifuged for 10 minutes at 10,000 g. For performing import experiments with mitochondria pretreated with proteinase K, the mitochondrial pellet was resuspended in MRM-S buffer prior to the experiment.

Cross-linking and dissociation of the cross-link bond—Sulfo-BSOCOES (Sulfo-Bis[2-(sulfosuccinimidooxy-carbonyloxy)ethyl]sulfone (Pierce)) from a 10-fold stock solution was added to obtain a final concentration of 10 mM. The cross-linker was added to either the mitochondrial-enriched fraction suspended in isotonic HIM buffer, or to purified mouse liver mitochondria suspended in MRM-S. After incubation for 30 minutes at room temperature, the cross-linker was quenched by the addition of 1 M Tris-HCl (pH 7.5) to a final concentration of 20 mM. After quenching, samples were lysed and Western blot-analyzed with the indicated antibody. Dissociation of the cross-link bond was performed according to the manufacturer's instructions. Briefly, the cross-linked product was incubated for 2 hours at 37° C. in alkaline conditions (0.1 M Na₂CO3, pH 11).

Western blot analysis and antibodies—Proteins were size-fractionated by SDS-PAGE, and then transferred to PVDF membranes (Immobilon-P, Bio-Rad). Western blots were developed by use of the enhanced chemiluminescence reagent (NEN). Purified recombinant histidine-tagged murine BID (Zha, J. et al., 2000. Science 290:1761-1765) was used as an immunogen to generate polyclonal anti-mBID Abs that were used for Western blotting. Additional antibodies included anti-mBAX Ab [651 (Wang, K. et al., 1996. Genes Dev. 10:2859-2869)], anti-BCL-X_{L} Ab (SC-634; Santa Cruz), anti-myc mAb (9E10; Santa Cruz), anti-HA mAb (3F10; Roche), anti-Cyt c (Cat. No. 556433; BD PharMingen, San Diego, Calif.).

Preparation of anti-Mtch2 Abs—Anti-Mtch2 Abs were prepared by subcutaneously injecting each of the peptides [aa93-106 (SEQ ID NO:29 for Ab1), aa110-127 (SEQ ID NO:30 for Ab2), and aa274-288 (SEQ ID NO:31 for Ab3)], conjugated to KLH (Imject Maleimide-activated mcKLH from Pierce, according to the manufacturer's protocol) in several places on the back of three white female rabbits. Two weeks after the injection, a 200 μg booster prepared with incomplete Freund's adjuvant (Sigma) was injected. Three weeks after boosting, blood was collected and incubated for two hours at room temperature, before being stored for overnight at 4° C. to enable clotting of the red blood cells. After the clot was removed, the serum was centrifuged for 30 minutes at 1500 g. The supernatant which resulted was then divided into aliquots, and stored at −20° C.

FAS induced apoptosis assay—Cells are treated with Fas ligand (100 nM together with 1 μg/ml cyclohexamide). At the indicated time points, cells were collected and cell death was monitored by FACScan using propidium iodide (PI) dye exclusion.

Additional information regarding the materials and methods used by the present invention can be found in Grinberg et al., 2002 (Supra) and 2005 (Supra).

### Example 1

### Identification of MTCH2 in a 45 kDa tBID-Containing Complex

The pathogenesis of numerous lethal and/or highly debilitating diseases is associated with disregulated, excessive or insufficient apoptosis. One approach for controlling apoptosis involves identifying molecules whose interaction with the key apoptotic mediator tBID is involved in mediating apoptosis. Once identified, the expression levels or activity of such molecules can be modulated to thereby regulate apoptosis. However, to date, no satisfactory methods of regulating apoptosis have been described. As described below, while reducing the present invention to practice, the present inventors have unexpectedly identified the key ligand of tBID for the tBID-induced remodeling of mitochondrial cristae of the mitochondrial apoptotic program.

Prior studies performed by the present inventors have shown that in TNFα-activated hematopoietic FL5.12 cells, tBID becomes part of a 45 kDa cross-linkable mitochondrial complex (FIG. 1 and Grinberg, M., et al., 2002, J. Biol. Chem. 277: 12237-12245), however the components of this complex were not yet identified.

Experimental Results

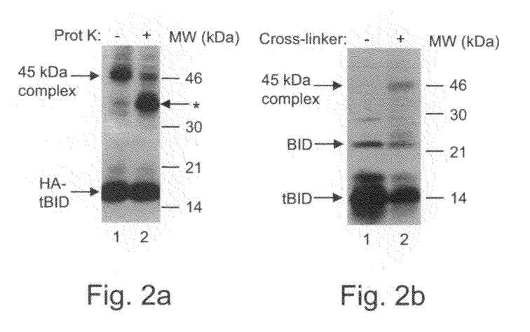

Formation of the 45 kDa tBID-containing complex relies on a surface-exposed mitochondrial protein—To determine whether the formation of the 45 kDa tBID cross-linkable complex requires the presence of a surface-exposed mitochondrial protein, purified and intact mouse liver mitochondria were incubated with a cytosolic extract prepared from 293T cells transfected with hemagglutinin (HA)-tagged tBID and prior to cross-linking the complex with BSOCOES the mitochondria were treated with proteinase K. As is shown in FIG. 2a, proteinase K pre-treatment led to a significant decrease in the intensity of the tBID-containing 45 kDa band, and the appearance of a new, ˜35 kDa BID-immunoreactive band. These results suggest that pre-treatment of naive mitochondria with proteinase K cuts off an exposed ˜10 kDa region from a ˜30 kDa mitochondrial protein, resulting in a 20 kDa fragment that cross-links with p15 tBID to form a ˜35 kDa complex. Thus, the 45 kDa BID-immunoreactive band most likely represents a complex between tBID and a ˜30 kDa surface-exposed mitochondrial protein.

BAX and BAK are not part of the tBID-containing 45 kDa complex—BAX and BAK are known to be essential downstream effectors of tBID, since Bax, Bak double knockout (DKO) mouse embryonic fibroblasts (MEFs) are resistant to tBID-induced apoptosis [Wei, M. C., 2001 (Supra); Zong W X, 2001 (Supra)]. It was previously demonstrated that tBID heterodimerizes with BAX or BAK to induce their oligomerization in the mitochondrial membrane [Eskes, R., et al., 2000, Mol. Cell. Biol. 20: 929-35; Wei MC, 2000 (Supra)]. In order to determine whether BAX and/or BAK are part of the 45 kDa complex or play an essential role in its formation, Bax and Bak DKO MEFs were infected with adenoviruses containing tBID (Ad-tBID). The heavy membrane fraction was then prepared, subsequently treated with cross-linker and subjected to tBID-Western blot analysis. As is shown in FIG. 2b, the 45 kDa cross-linked complex is formed in heavy membranes prepared from Ad-tBID-infected Bax and Bak DKO MEFs. Thus, these results indicate that BAX and BAK are not part of this complex, and are not essential to its formation.

The 45 kDa species represents a complex between tBID and Mtch2, a previously uncharacterized mitochondrial protein—In order to determine the components of the tBID-cross linkable complex, HA-tagged tBID fusion protein (pCA14-HA-tBID) was used to transfect 293T cells and the mitochondria-enriched heavy membrane fraction prepared from such cells was treated with cross-linker and then lysed. The mitochondrial lysate was then incubated with anti-HA antibody-coupled agarose beads, and the HA-tBID monomers/complexes were eluted by incubation in the presence of HA peptide. Initial results indicated that the anti-HA antibody column efficiently captured the 45 kDa complex, and that the HA peptide was efficient in releasing it from the column (data not shown). Next, the starting material for purification of the complex was scaled up so as to produce a quantity of putative 45 kDa tBID-containing complex visible by Coomassie blue staining (FIG. 3a). In this gel, HA-tBID was clearly visible as a protein species of about 15 kDa whose identity as such was further confirmed by mass spectrometry. A small portion of the eluted material was also taken for Western blot analysis using anti-HA Abs. As is shown in FIG. 3b, the eluted Coomassie blue band was tBID positive. Mass spectrometry analysis of the 45 kDa species revealed that the 45 kDa complex included the two tBID-derived peptides IEPDSESQEEIIHNIA (SEQ ID NO:3) and HLAQIGDEMDHNIQPTLVR (SEQ ID NO:4; FIG. 3c), and, unexpectedly, the peptide VLIQVGYEPLPPTIG (SEQ ID NO:5) derived from human mitochondrial carrier homolog 2 (Mtch2), an uncharacterized 33.4 kDa protein (FIG. 3c).

Thus, these findings demonstrate that the 45 kDa BID-immunoreactive complex represent a complex between tBID and the human mitochondrial carrier homolog 2 (Mtch2).

### Example 2

### Affinity Tagged tBID and Mtch2 are Capable of Forming a ˜48 kDa Complex In Vitro

Mtch2 is a novel and previously uncharacterized 33 kDa protein first identified in a screen for human cDNAs, and which was named after the conserved mitochondrial carrier domain (MCD) it contains (Zhang, Q. H., et al., 2000, Genome Res. 10: 1546-1560). To determine if recombinant Mtch2 and tBID are capable of forming a cross-linkable complex, Mtch2 was first cloned and affinity tagged tBID and Mtch2 were expressed in 293T cells, as follows.

Experimental Results

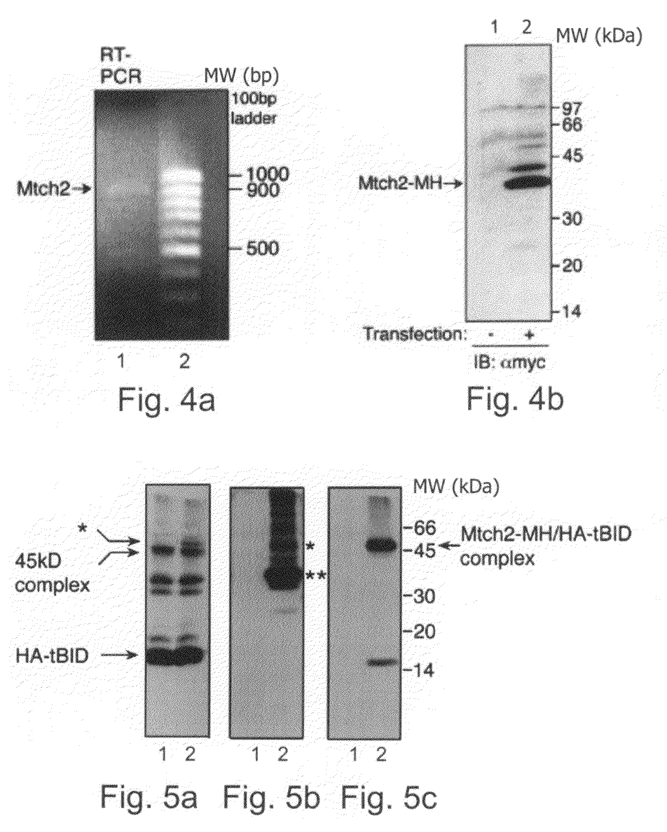

Construction of myc and His-tagged Mtch2 (Mtch2-MH)— To generate recombinant affinity-tagged Mtch2 for experiments investigating the site of interaction of tBID with Mtch2, the human Mtch2 gene was cloned from total RNA prepared from HeLa cells. Agarose-gel analysis indicated that the RT-PCR reaction using specific primers to the Mtch2 gene (SEQ ID NOs:1 and 2) resulted in a single transcript of 900 bp (FIG. 4a). To express recombinant myc- and His-tagged Mtch2 fusion protein, the Mtch2 cDNA was cloned into the pcDNAIII.1-myc-His construct (pcDNAIII.1-Mtch2-myc-His) and further expressed in 293T cells. Western blot analysis of transfectants using anti-myc antibodies detected a single major protein species of the expected 36 kDa molecular weight (Mtch2-MH; FIG. 4b).

Co-expression of tBID (induced by tetracycline) and Mtch2 revealed a novel protein species of about 48 kDa—To assess whether tBID interacts with Mtch2 at the mitochondrion, Mtch2-MH and HA-tBID were co-expressed in 293T cells. Since co-expression of Mtch2-MH with HA-tBID in normal 293T cells resulted in very low expression levels of Mtch2-MH (data not shown), 293T-T-Rex cells (Invitrogen) were transiently transfected with pCA14-HA-tBID to generate cell line T-Rex-HA-tBID for tetracycline-inducible expression of tBID alone, and T-Rex-HA-tBID cells were transfected with pcDNAIII.1-Mtch2-myc-His for co-expression of Mtch2-MH and tetracycline-inducible HA-tBID. For inducing HA-tBID expression in these cell lines, 18 hours post-transfection the transfectants were incubated for 5 hours with the tetracycline doxycyclin. Mitochondria-enriched heavy membrane fractions prepared from these cells were treated with cross-linker, lysed, and analyzed by Western blot using anti-HA antibodies (FIG. 5a). Western blot analysis using anti-HA antibodies detected a novel protein species of about 48 kDa in cells expressing both proteins, but not in cells expressing only HA-tBID (FIG. 5a). To determine whether this species indeed represented a complex between HA-tBID and Mtch2-MH, the mitochondrial lysates were loaded onto nickel columns and the eluted proteins were separated by SDS-PAGE and analyzed by Western blot using either anti-myc (FIG. 5b) or anti-HA (FIG. 5c) antibodies. As expected, Mtch2-MH bound to the nickel column was detected in the eluted material using anti-myc antibodies (FIG. 5b, lane 2). Western blot analysis using anti-HA antibodies clearly indicated that the approximately 48 kDa species that appeared in lanes 2 of FIG. 5a and in lane 2 of FIG. 5b represent a complex between HA-tBID and Mtch2-MH (FIG. 5c, lane 2).

Altogether, these results demonstrate that co-expression of the affinity-tagged tBID and Mtch2 proteins reveals the formation of a cross-linkable ˜48 kDa protein complex as expected from using affinity tagged proteins.

### Example 3

### tBID Destabilizes Mtch2

To further characterize the biochemical properties of Mtch2, Mtch2-MH was expressed alone or with HA-tBID in 293T cells, and biochemical assays were performed on the mitochondria-enriched fractions of transfectants.

Experimental Results

Proteinase K treatment reduces the size of an Mtch2-containing protein complex—In mitochondria prepared from Mtch2-MH-expressing cells treated with proteinase K, Mtch2-MH showed a reduction in molecular weight of about 10 kDa (i.e., from about 35 kDa to about 25 kDa; FIG. 6a, lane 2). This reduction in size is similar to the reduction observed with the tBID-cross-linked complex using mouse liver mitochondria pretreated with proteinase K (FIG. 2a). Thus, these results suggest that mouse Mtch2 is the protein in liver mitochondria that cross-links to tBID. Proteinase K treatment was also performed on mitochondria prepared from cells expressing both Mtch2-MH and HA-tBID and it was found that the presence of HA-tBID did not alter the proteinase sensitivity of Mtch2-MH (FIG. 6a, lane 4).

Mtch2 is a surface-exposed integral membrane protein destabilized by tBID In further experiments, the mitochondria were treated with urea, which releases all except for the most integral membrane proteins. Following treatment, the membranes were separated from the soluble fraction by centrifugation and both urea-soluble and -insoluble protein fractions were analyzed by Western blot using anti-myc antibodies. The results clearly demonstrate that when using mitochondria prepared from cells expressing Mtch2-MH but not HA-tBID, Mtch2-MH was largely resistant to urea extraction (FIG. 6b, lanes 1 and 2). In contrast, when using mitochondria prepared from cells expressing both Mtch2-MH and HA-tBID, Mtch2-MH was more sensitive to urea extraction (FIG. 6b, lanes 3 and 4). Thus, these results demonstrate that the presence of tBID at the mitochondria destabilizes the membrane-attachment of Mtch2-MH.

These experiments established that tBID binds Mtch2 integrally within the mitochondrial membrane in such a way as to destabilize membrane attachment of Mtch2.

### Example 4

### Sequence Comparison Between Mtch2 and the Mitochondrial Carrier Protein Family

The mitochondrial carrier protein (MCP) family comprises a variety of proteins that catalyze the exchange of substrates across the IMM (Walker, J. E. and Runswick, M. J. 1993, J. Bioenerg. Biomembr. 25: 435-446). All MCP family members are relatively small proteins, with a molecular mass ranging from 28 to 34 kDa in size. Comparison of the amino acid sequences of the different carriers has shown that they are made up of three tandem repeats, each about 100 amino acids in length and known as the mitochondrial carrier domain (MCD). Each repetitive element contains two hydrophobic stretches that are of sufficient length to span the membrane as α-helices, separated by an extensive hydrophilic region. Based on this information, it was proposed that the overall structure of this protein family consists of six transmembrane α-helices, in which both the N- and the C-termini of the proteins are facing the IMS, and the three long hydrophilic segments (connecting the two transmembrane regions of each domain) are facing the matrix (Palmieri, F. 1994, FEBS Lett, 346: 48-54). From the functional characterization of several different carriers that were reconstituted in liposomes, it would appear that the mitochondrial carriers are not only similar in structure but also in function, and are characterized by a common kinetic mechanism [Palmieri, 1994 (Supra)].

To further understand the function of the Mtch2 protein, the amino acid sequence of Mtch2 was aligned with various MCP protein members and Mtch2 homologues using the Standard protein-protein BLAST [blastp] software of the NCBI, as follows.

Bioinformatic Results

Mtch2 shares amino acid sequence homology with members of the MCP family of molecules—Mtch2 was named after the conserved mitochondrial carrier protein domain (MCD) included therein. This conserved 100-residue domain appears in members of the mitochondrial carrier protein (MCP) family which comprises various types of inner mitochondrial membrane substrate carrier proteins, such as ADP/ATP translocase [Palmieri, 1994 (Supra)], involved in transfer of energy metabolites across the inner mitochondrial membrane. The MCD is composed of two transmembrane regions connected by a linker region. FIG. 7a shows a multiple amino acid sequence alignment of five proteins with known function most similar to human Mtch2. These are: human carnitine/acylcarnitine translocase (CAC), yeast RIM2, bovine citrate transport protein (CTP), mouse mitochondrial uncoupling protein 2 (UCP2), and human mitochondrial uncoupling protein 1 (UCP1). The NCBI GenInfo Identifier (gi) sequence identification numbers of these sequences are: human Mtch2, 7657347 (SEQ ID NO:7); human carnitine/acylcarnitine translocase (CAC), 3914023 (SEQ ID NO:9); yeast RIM2, 585856 (SEQ ID NO:10); bovine citrate transport protein (CTP), 2497986 (SEQ ID NO:11); mouse mitochondrial uncoupling protein 2 (UCP2), 2497982 (SEQ ID NO:12); and human mitochondrial uncoupling protein 1 (UCP1), 1351353 (SEQ ID NO:13). As is shown in FIG. 7a, all MCP family members have three MCDs, whereas Mtch2 has only one. Based on this and other criteria (see below), Mtch2 proteins seem to be distinct from MCPs. Moreover, Mtch2 has several close relatives, which together form a separate subfamily. In chordates such as mammals and birds the Mtch family includes two members, Mtch1 and Mtch2, while in fish and tunicates there is apparently only one Mtch2 ortholog. Among invertebrates, C. elegans has one, and D. melanogaster has two, Mtch proteins. All family members are predicted to have a single MCD domain. Mtch2 proteins form a clear sub-group within the MCP family and can be aligned across almost their entire length. FIG. 7b shows alignment of human Mtch2 with related Mtch-family proteins from various species. As is shown in FIG. 7b, the Mtch2 protein whose amino acid sequence has the lowest identity to that of human Mtch2 is zebrafish Mtch2, with only 69% identity, as determined by the Standard protein-protein BLAST [blastp] software of the NCBI. Mtch2 proteins are predicted to contain an N-terminal mitochondrial targeting sequence and three transmembrane domains, in which the first two are part of the MCD (FIGS. 7a-b). Bioinformatic analysis suggested that the closest relative of Mtch2 in the MCP family is brown fat uncoupling protein (UCP), which dissipates oxidative energy into heat by transporting protons from the mitochondrial intermembrane space into the mitochondrial matrix. A search for the nearest homolog to human Mtch2 using Standard protein-protein BLAST [blastp] software of the NCBI revealed that the polypeptide most similar to human Mtch2 is bovine (Bos taurus) Mtch2 (gi/28603744), having an amino acid sequence 94% similar to that of human Mtch2. The amino acid sequence of human Mtch2 (NCBI gi/7657347) was searched against the NCBI Conserved Domain Database (CDD) version 1.60 using the RPS-BLAST program (Altschul, S. F. et al., 1997. Nucleic Acids Res. 25:3389-3402). The segment corresponding to amino acid residues 128-192 of human Mtch2 were found to correspond to the mitochondrial carrier protein (MCP) domain (CDD|9082) with an e-value of 2⁻⁸ (53.8 bits).

Mtch1 (or presenilin-1-associated protein; PSAP) known to induce apoptosis is 48% homologous to Mtch2—A recent study has reported that the novel presenilin-1-associated protein (PSAP) is homologous to the MCP family [Xu X, 2002, J. Biol. Chem. 277:48913-48922]. The amino acid sequence of PSAP was aligned to that of the Mtch family members and was found to be identical to that of Mtch1. Amino acid sequence alignment indicated that the percent identity between human Mtch1/PSAP and human Mtch2 is 48% (FIG. 7c). Mtch1/PSAP has been reported to localize to the mitochondria and its expression in HEK293 cells induces apoptosis, which is accompanied by Cyt c release and caspase-3 activation [Xu X, 2002, (Supra)].

Altogether, these analyses revealed that Mtch2 contains a mitochondrial carrier protein domain (MCD) found in mitochondrial carrier proteins (MCPs) which catalyzes transport of metabolites across the inner mitochondrial membrane. Mtch2 was found to belong to a distinct subfamily of MCPs aligned across almost their entire length, and containing an N-terminal mitochondrial targeting sequence and three transmembrane domains, in which the first two are part of the MCD.

### Example 5

### Exogenous Mtch2 and tBID Associate in Mitochondria

Experimental Results

Mtch2 co-localizes with tBID at the mitochondria—To confirm that Mtch2 localizes to the mitochondria, the cloned human Mtch2 cDNA was fused it to green fluorescent protein (GFP) and was used to transiently transfect HeLa cells. HeLa cells transfected with the Mtch2-GFP chimeric protein were incubated with MitoTracker Red (MTR; to label mitochondria) and were analyzed by means of confocal microscopy. As is shown in FIGS. 8a-c, a major part of the chimeric Mtch2-GFP molecules co-localized with MTR, confirming their mitochondrial localization. Similarly, transfection of HeLa cells with either tBID alone or tBID together with Mtch2-GFP and overlays of the stained images revealed co-localization of tBID with MTR (FIGS. 8d-f) and tBID and Mtch2-GFP (FIG. 8g-i).

These studies demonstrate that Mtch2 co-localizes with tBID at the mitochondria.

### Example 6

### Mtch2 is an Integral Membrane Protein Exposed on the Surface of Mitochondria

To further characterize the biochemical properties of endogenous human and mouse Mtch2, the present inventors have raised three polyclonal antibodies to three separate peptides in mouse Mtch2, as follows.

Experimental Results

Preparation of anti-Mtch2 antibodies—The polyclonal antibodies were raised against the following peptides: SEQ ID NO:29 (amino acids 93-106 as set forth in SEQ ID NO:17; AAD52647 for the Ab1 antibodies), SEQ ID NO:30 (amino acids 110-127 as set forth in SEQ ID NO:17 for the Ab2 antibodies), and SEQ ID NO:31 (amino acids 274-288 as set forth in SEQ ID NO:17 for the Ab3 antibodies). These peptides appear in blue boxes in FIG. 7c. The first two mouse peptides are very similar to their human counterparts (corresponds to amino acids 93-106 and 110-127 of SEQ ID NO:7 (GenBank Accession No. NP_{—}055157), respectively; and the third mouse peptide is identical to the human peptide (amino acids 274-288 of SEQ ID NO:7).

Characterization of the anti-Mtch2 antibodies (Abs) of the present invention—To examine the ability of the anti-Mtch2 polyclonal Abs to recognize Mtch2, Western blot analysis was performed on mouse and human cells. As is shown in FIGS. 9a-c, all three antibodies recognized endogenous mouse Mtch2 from liver mitochondria, whereas only Ab2 and Ab3 recognized human Mtch2, exogenously expressed in 293T cells. In addition, Ab3 and, to a lesser extent, Ab2 also recognized endogenous human Mtch2 in 293T cells.

Mtch2 is an integral membrane protein resistant to both alkali and urea treatments—To assess whether Mtch2 is an integral membrane protein, intact mouse liver mitochondria were treated with either alkali or urea, two treatments which release all except for the most integral membrane proteins. Following treatment, the membranes were separated by centrifugation from the soluble fractions and the membrane fractions were analyzed by Western blot using anti-Mtch2 Ab2 antibodies. As is shown in FIG. 9d, Mtch2 is largely resistant to both alkali and urea extraction, indicating that it is an integral membrane protein.

Mtch2 is sensitive to proteinase K treatment—Since the results shown in FIG. 2a and in Example 1, hereinabove demonstrated that the 45 kDa complex represents an association between tBID and a mitochondrial protein that is sensitive to proteinase K, the present inventors have further examined the sensitivity of Mtch2 to proteinase K. To this end mouse liver mitochondria were treated with increasing concentrations of proteinase K and the treated proteins were subjected to Western blot analyses using the Mtch2 antibodies of the present invention (Ab1, Ab2, and Ab3). As is shown in FIGS. 9e-g, low concentration of proteinase K (0.1 μg/ml) resulted in the cleavage of Mtch2 into several fragments: a ˜10 kDa fragment recognized by Ab1 (FIG. 9e, lane 2), a 20 kDa fragment and a ˜10 kDa fragment recognized by Ab2 (FIG. 9f, lane 2), and a ˜20 kDa fragment and a ˜10 kDa fragment recognized by Ab3 (FIG. 9g, lane 2). Treatment of mitochondria with a tenfold-higher concentration of proteinase K (i.e., 1 μg/ml) resulted in a decrease in the intensity of the ˜20 kDa fragment and in an increase in the intensity of ˜10 kDa fragment recognized by Ab2 and Ab3 (FIGS. 9f and g, lane 3).

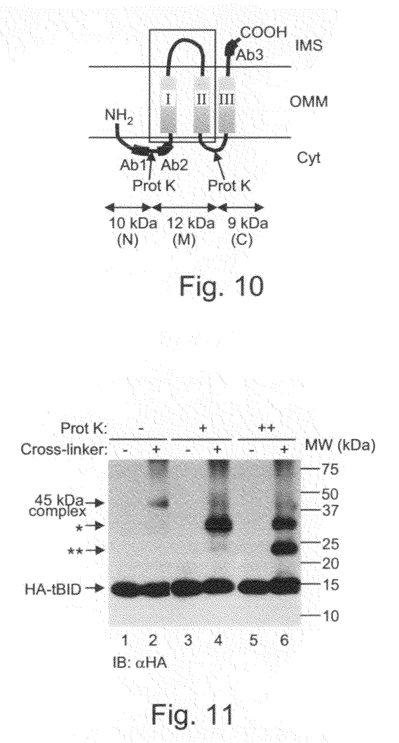

Identification of proteinase K cleavage sites in Mtch2—FIG. 10 presents a schematic model including the putative proteinase K cleavage sites in Mtch2: at lysine 100 or 111 (positioned in a hydrophilic loop between the peptides used to generate Ab1 and Ab2; the exact position of these lysines is marked in FIG. 7c) and at lysine 219 (positioned in a hydrophilic loop connecting TMII and TMIII). Cleavage at these two sites results in the generation of three fragments: a ˜10 kDa N-terminal fragment (N), a ˜12 kDa middle fragment (M), and a ˜9 kDa C-terminal fragment (C). Thus, lysines 100/111 and 219 (and the areas surrounding them) are most likely exposed on the surface of mitochondria, and therefore all three TM domains are likely to span the outer mitochondrial membrane.

tBID cross-links to the C-terminal fragment of Mtch2—The ˜10 kDa reduction in size of Mtch2 following treatment with the low concentration of proteinase K is similar to the reduction in size from ˜45 kDa to ˜35 kDa that was observed with the tBID cross-linked complex (FIG. 2a). Thus, tBID cross-links to the middle +C-terminal fragment, and not to the N-terminal fragment. To determine whether tBID cross-links to both the middle and C-terminal fragments or only to one of them, mitochondria were pre-treated with a tenfold-higher concentration of proteinase K (which results in the generation of separate middle and C-terminal fragments; see FIGS. 9e-g) prior to the addition of HA-tBID and cross-linker. Pre-treatment with the higher concentration of proteinase K led to a decrease in the intensity of the ˜35 kDa cross-linked band, and the corresponding appearance of a new HA-immunoreactive band, which migrated slightly below the 25 kDa marker (FIG. 11, lane 6). These results suggest that tBID cross-links to the C-terminal fragment (15+9=24 kDa) rather than the middle fragment (15+12 =27 kDa) of Mtch2.

Altogether, these results demonstrate the generation of anti-Mtch2 polyclonal Abs and the characterization of the tBID-interacting domain on the Mtch2 protein.

### Example 7

### The 45 kDa Complex formed in TNFα-Treated FL5.12 Cells is Composed of tBID and Mtch2

Prior studies performed by the present inventors have demonstrated that the 45 kDa cross-linked complex appears in TNFα-treated, but not in untreated (-TNFα), FL5.12 cells [Grinberg, M., 2002 (Supra)]. To assess whether the 45 kDa band detected in TNFα-treated FL5.12 cells indeed represents a complex between endogenous tBID and Mtch2, the cross-linker BSOCOES, which can be reversed in alkaline conditions, was employed, as follows.

Experimental Results

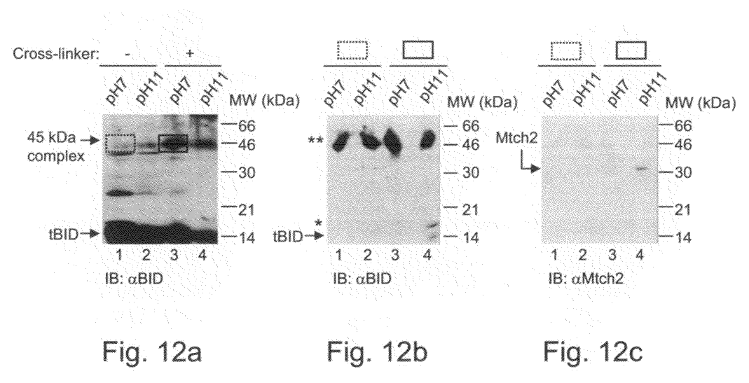

The tBID-containing complex formed in TNFα-treated FL5.12 cells is sensitive to alkaline conditions—FL5.12 cells were treated for 5 hours with TNFα/CHX following which the heavy membrane fraction was treated with the BSOCOES cross-linker or was remained untreated, and was further incubated in either neutral (pH 7) or alkaline (pH 11) conditions. At the end of the incubation, the membranes were lysed and subjected to Western blot analysis using anti-BID antibodies. As is shown in FIG. 12a, alkaline conditions significantly reduced the intensity of the 45 kDa band.

The endogenous 45 kDa complex formed in TNFα-treated FL5.12 cells is composed of tBID and Mtch2—To further characterize the endogenous 45 kDa complex formed in TNFα-treated FL5.12, the experiment described hereinabove and in FIG. 12a was repeated and the gel sections corresponding to the position of the 45 kDa complex in lanes 1 and 3 (marked by boxes in FIG. 12a) were excised, and incubated in either neutral or alkaline conditions. The sections were then layered onto a 15% denaturing gel, and the proteins were resolved by SDS-PAGE. Western blot analysis using either anti-BID antibodies (FIG. 12b) or anti-Mtch2 antibodies (Ab2; FIG. 12c) indicated that tBID and Mtch2 appear exclusively in the gel section that represents mitochondria treated with cross-linker and incubated in alkaline conditions. Altogether, these results strongly suggest that the endogenous 45 kDa complex formed in TNFα-treated FL5.12 cells is sensitive to alkaline conditions and is composed of tBID and Mtch2.

### Example 8

### Activation with TNFα Recruits tBID and Bax to the 185 kDa Resident Mitochondrial Complex

To verify the interaction between endogenous tBID and Mtch2 in mitochondria of FL5.12 cells signaled to die by TNFα, the blue-native polyacrylamide gel electrophoresis (BN-PAGE) (Schagger, H. 2001, Methods Cell Biol. 65: 231-44) method was employed. This method has been proven powerful for purifying the respiratory chain complexes.

To this end, mitochondria purified from FL5.12 cells were either treated for 5 hours with TNFα/CHX or remained untreated. Purified mitochondria were solubilized with n-dodecyl β-D-maltoside (DM), centrifuged and the solubilized complexes were mixed with Coomassie blue G-250. The resulting complexes were resolved on a 5-13% gradient gel under non-denaturing conditions (“1^{st} D BN-PAGE”). These native complexes were separated into their individual components by placing a native gel slice horizontal as a stack above an SDS-denaturing gel (“2^{nd} D SDS-PAGE”).

Experimental Results

tBID and Mtch2 reside in a ˜185 kDa mitochondrial complex in TNFα-induced FL5.12 cells —FIGS. 13a-c depict 2-D-gel analysis of FL5.12 cells treated for 5 hours with TNF-α. Coomassie blue staining of the 2-D-gel (shown in FIG. 13a) revealed the individual protein constituents of each native complex. The stained gel was then transferred to nitrocellulose and immunoblotted with either anti-Mtch2 (Ab2) or anti-BID antibodies. As can be seen in FIGS. 13b-c, in mitochondria prepared from TNFα-treated FL5.12 cells, tBID appeared in two spots (spots Nos. 1 and 2), and one of them (spot No. 1) co-migrated with the Mtch2 smeared spot. Both tBID and Mtch2 were detected on the 2^{nd} dimension gel at higher molecular weights than expected, since the migration of proteins from the horizontal native gel slice to the SDS-gel takes longer than the migration of soluble proteins that are directly loaded onto the SDS-gel [compare the positions of the tBID and Mtch2 spots in the large blots, to the tBID and Mtch2 bands in the “DM-ext” lanes (FIGS. 13b-c)].

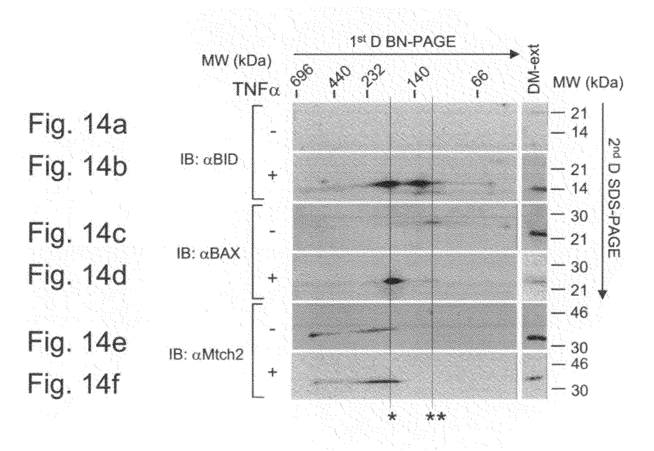

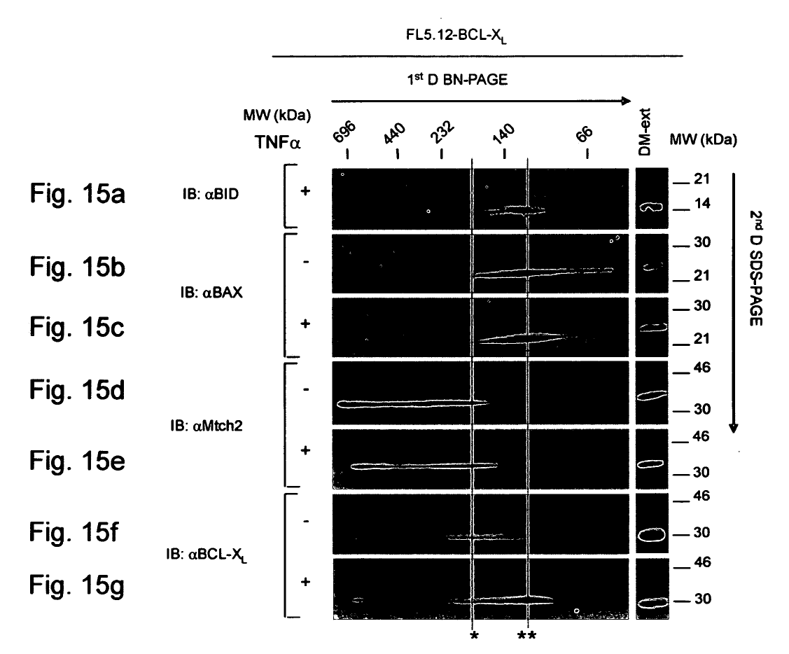

tBID is not part of the 185 kDa mitochondrial resident complex in viable FL5.12 cells—The blue-native (BN) 2-D-gel analysis was repeated on TNFα-induced (FIGS. 14b and f) or uninduced (FIGS. 14a and e) FL5.12 cells and the blot was reacted with the anti-BID or anti-Mtch2 (Ab2) antibodies. As is shown in FIGS. 14a and 14e, in mitochondria prepared from viable FL5.12 cells (i.e., TNFα-uninduced cells), Mtch2 was detected in a smeared spot that ranged from approximately 185 kDa to 230 kDa in the native gel (FIG. 14e), whereas tBID, as expected, was not detected (FIG. 14a).

Altogether, these results strongly suggest that Mtch2 resides in a relatively large mitochondrial complex in viable FL5.12 cells, and that activation with TNFα leads to the recruitment of tBID to this complex.

The 185 kDa mitochondrial resident complex in TNFα-induced FL5.12 cells includes the BAX protein—To identify additional components of the approximately 185 kDa complex, individual protein spots that aligned with Mtch2 and tBID were excised from the 2^{nd} dimension gel and sequenced by mass spectrometry. By sequencing two of the spots (one at approximately 35 kDa and the other at 25 kDa) two peptides were identified. The approximately 35 kDa protein spot included a peptide derived from the mouse Mtch2 protein (corresponding to amino acids 26-41 as set forth in SEQ ID NO:17); and the 25 kDa protein spot included a peptide derived from the mouse BAX protein (corresponding to amino acids 66-78 as set forth in SEQ ID NO:26; GenBank Accession No. NP_{—}031553). These results demonstrate that in FL5.12 cells which are signaled to die by TNFα the ˜185 kDa mitochondrial complex includes the Mtch2 and BAX proteins.

Activation by TNFα also recruits BAX to the Mtch2-resident complex—To confirm the presence of BAX in this complex, TNFα-induced or uninduced FL5.12 cells were subjected to the blue-native (BN) 2-D-gel followed by Western blot analysis using the anti-BAX (FIGS. 14c-d) or anti-Mtch2 (Ab2) (FIGS. 14e-f) antibodies. As is shown in FIGS. 14c-f, in mitochondria prepared from viable FL5.12 cells, BAX was detected in a single spot that migrated at approximately 115 kDa in the native gel (FIG. 14c), whereas Mtch2 was detected in a smeared spot that ranged from approximately 185 kDa to 230 kDa in the native gel (FIG. 14e). Strikingly, in TNFα-treated FL5.12 cells, the position of BAX was seen to shift to a single spot that co-migrated at approximately 185 kDa in the native gel (FIG. 14d) similar to one of the two tBID spots (FIG. 14b). On the other hand, TNFα had no significant effect on the position of Mtch2.

Altogether, these results demonstrate that TNFα leads to the recruitment of both tBID and BAX to the Mtch2-resident complex.

### Example 9

### BCL-XL Partially Inhibits the Recruitment of both BAX and tBID to the Mtch2-Resident Complex

Prior studies performed by the present inventors demonstrated that FL5.12 cells that stably express BCL-X_{L} (FL5.12-BCL-X_{L}) do not release Cyt c from the mitochondria in response to TNFα [Gross A, 1999 (Supra)]. Since the recruitment of tBID and BAX to the Mtch2 complex might be related to Cyt c release, FL5.12-BCL-X_{L} cells were utilized to assess whether the presence of BCL-X_{L} affects this recruitment. Briefly, FL5.12-BCL-X_{L} cells were induced for 5 hours with TNFα/CHX (+) or were remained un-induced, and mitochondria prepared from these cells were subjected to the (BN) 2-D-gel analysis using anti-BID (FIG. 15a), anti-BAX (FIGS. 15b-c), anti-Mtch2 (Ab2; FIGS. 15d-e) and anti- BCL-X_{L} (FIGS. 15f-g).

Experimental Results

Overexpression of BCL-X_{L} inhibits the recruitment of both tBID and BAX to the Mtch2-resident complex—In viable FL5.12-BCL-X_{L} cells (i.e., un-induced cells), BAX was detected in a smeared spot that ranged from approximately 66 kDa to 185 kDa in the native gel (FIG. 15b), whereas BCL-X_{L} was detected in a smeared spot that ranged from approximately 115 kDa to 230 kDa in the native gel (FIG. 15f), and partially co-migrated with the smeared spot of Mtch2 (FIGS. 15d). Strikingly, in TNFα-treated FL5.12-BCL-X_{L} cells, the position of BAX did not shift (compare FIGS. 15b and c), and tBID now appeared in a smeared spot (FIG. 15a) that co-migrated with BAX. On the other hand, the addition of TNFα did induce a shift in the position of BCL-X_{L} (FIG. 15g), which now co-migrated with BAX and tBID. These results demonstrate that overexpression of BCL-X_{L} inhibits the recruitment of both tBID and BAX to the Mtch2-resident complex.

To confirm these results, the mitochondria prepared from FL5.12-BCL-X_{L} cells, which were either induced with TNFα/CHX or remained un-induced, were further subjected to cross-linking experiments with the BSOCOES cross-linker.

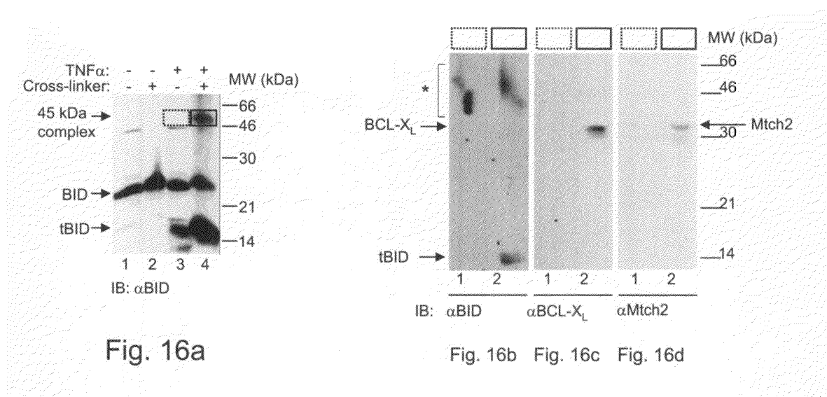

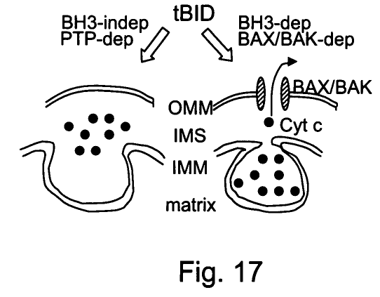

tBID forms two ˜45 kDa protein complexes in TNFα-treated FL5.12-BCL-X_{L} cells: the tBID and BCL-X_{L} complex and the tBID and Mtch2 complex—As is surprisingly shown in FIG. 16a, when TNFα-induced FL5.12-BCL-X_{L} cells were treated with cross-linker, a 45 kDa BID-immunoreactive band was detected. To assess whether this band represented a complex between tBID (15 kDa) and BCL-XL (30 kDa), the experiment was repeated and the gel sections corresponding to the position of the 45 kDa band in lanes 3 and 4 (marked by boxes in FIG. 16a) were excised, incubated in alkaline conditions, and later resolved as described in Example 7, hereinabove and in FIGS. 12a-c. Western blot analysis using either anti-BID antibodies or anti-BCL-X_{L} antibodies indicated that tBID (FIG. 16b) and BCL-X_{L} (FIG. 16c) appear exclusively in the gel section that represents mitochondria treated with cross-linker and incubated in alkaline conditions. To assess whether this 45 kDa gel section might also contain a complex comprised of tBID and Mtch2, Western blot analysis was performed using anti-Mtch2 Ab2 antibodies and found that Mtch2 was also released from this gel section (FIG. 16d).

Taken together, these results suggest that the 45 kDa BID-immunoreactive band detected in TNFα-treated FL5.12-BCL-X_{L} cells represents two complexes: One composed of tBID and BCL-X_{L}, and the other composed of tBID and Mtch2. Thus, overexpression of BCL-X_{L} increases the formation of a tBID-BCL-X_{L} complex and thus partially inhibits the recruitment of tBID to the Mtch2-resident complex.

### Example 10

### Down-Regulation of Mtch2 Expression Accelerates TNFα And Fas-Induced Apoptosis

Down-regulation (DR) the expression of a given protein can help to determine it's function and significance to a certain process. To examine the role of Mtch2 in mitochondria, and specifically, in the tBID-death pathway the present inventors employed the RNAi approach. For this purpose, the pRETRO-SUPER plasmid (Brummelkamp, T. R., et al., 2002, Cancer Cell, 2: 243-247), which enables the transcription of a short double stranded RNA in the form of stem and loop (shRNA; Brummelkamp, T. R., et al., 2002, Cancer Cell, 2: 243-247) was employed. Several Mtch2 specific sequences were designed, and only one, named 370 [CTCGAGAGATGATCGCTCG (SEQ ID NO:27) which corresponds to nucleic acid 374-393 of SEQ ID NO:28 of the human Mtch2 RNA (GenBank Accession No. NM_{—}014342)] was successful in significantly reducing the expression level of endogenous human Mtch2 in HeLa cells. To this end, the pRETRO-SUPER 370 plasmid was utilized to generate single stable clones.

HeLa cells stably transfected with the pRETRO-SUPER 370 plasmid exhibit reduced Mtch2 expression—Stable clones expressing the Mtch2 RNAi (pRETRO-SUPER 370; SEQ ID NO:28) or the pRETRO-SUPER empty vector were subjected to Western blot analyses using the Mtch2 Ab2 antibodies (FIG. 18a) or a β-tubulin antibody (Sigma, St Louis, Mo., USA) (FIG. 18b). The results show that in Mtch2 DR HeLa cells the expression of Mtch2 is significantly reduced as compared to control HeLa cells transfected with the empty vector (pRETRO-SUPER) (FIG. 18a). On the other hand, both Mtch2 DR and control HeLa cells express similar levels of tubulin (FIG. 18b).

Mtch2 DR HeLa cells exhibit higher rate of Fas-induced apoptosis—To determine the effect of downregulating the expression level of Mtch2 on either TNFα or Fas-induced apoptosis, time course apoptosis studies were performed using two of the Mtch2 DR clones, one of the empty vector clones, and parent HeLa cells. As is shown in FIG. 18c, Fas-induced apoptosis is accelerated in both the Mtch2 DR clones compared to the vector/control and parent cells. Similar results were obtained with TNFα (data not shown).

Mtch2 DR results in elimination of the 45 kDa tBID-Mtch2 complex—As is described hereinabove and in Grinberg M., et al. (Mol. Cell. Biol. 2005, 25: 4579-90), the 45 kDa Mtch2-tBID complex is formed by incubating purified, intact mouse liver mitochondria with a cytosolic extract prepared from 293T cells transfected with HA-tBID. To determine whether downregulating Mtch2 has an effect on tBID targeting and/or formation of the 45 kDa complex, mitochondria prepared from control and Mtch2 downregulated (DR) HeLa cells were incubated with 293T cells cytosol containing HA-tBID. After 30 minutes of incubation, mitochondria were separated from the soluble fraction by centrifugation, either treated or not with cross-linker, and lysed. Western blot analysis using anti-HA antibodies (recognizing HA-TBID) demonstrated that in mitochondria prepared from Mtch2 DR cells there was less targeting of tBID to mitochondria compared to mitochondria from control cells, and significantly less formation of the 45 kDa complex (FIG. 19a). These results suggest that Mtch2 is important for targeting of tBID to mitochondria.

Mtch2 downregulation results in acceleration of tBID-induced Cyt c release—As mentioned before, the most established effect of tBID on the mitochondria is the release of Cyt c. To examine whether Mtch2 DR has an effect on tBID-induced Cyt c release from purified mitochondria, recombinant tBID (Zha, J. et al., 2000. Science 290:1761-1765) was incubated with mitochondria prepared from control or Mtch2 DR HeLa cells. Following incubation, the mitochondria were separated by centrifugation from the soluble fraction and the soluble fraction was further analyzed by Western blot using anti-Cyt c antibodies. As is shown in FIG. 19b, there is significant increase in the release of Cyt c from mitochondria prepared from Mtch2 DR HeLa cells than from mitochondria prepared from control HeLa cells. In addition, this increase was also correlated with increased incubation time with recombinant tBID [e.g., from 1 minute (lane 2) to 5 minutes (lane 4)].

Altogether, these surprising results demonstrate that Mtch2 is involved in controlling/limiting tBID-induced Cyt c release and that downregulation of Mtch2 results in increased rates of Fas-induced apoptosis and Cyt c release.

### Example 11

### Generation of Mtch2 Knock-Out Mice

The experiments described above using Mtch2 DR cells provided clues to the importance of Mtch2 for normal mitochondrial function and for tBID-induced mitochondrial apoptosis. If the presence of Mtch2 is essential for certain aspects of mitochondrial function, and its interaction with tBID is essential for certain aspects of mitochondria apoptosis, it will be important to examine these aspects in a complete Mtch2-null background. An Mtch2-null (knock-out) mouse can be a valuable tool to test the necessity of Mtch2 to normal function of mitochondria and to the mitochondrial apoptotic program in vivo.

Experimental Results

Mtch2 is mostly expressed in the heart—To characterize the expression pattern of Mtch2, various tissues derived from seven-week old wild-type C57BL/6 female mice were subjected to SDS-PAGE, followed by Western blot analysis using anti-Mtch2 Abs (Ab2). As is shown in FIG. 20, Mtch2 is highly expressed in the heart and the liver and to a lesser extent also in the brain, intestine and kidney. These results suggest that Mtch2 might be involved in the normal development and function of such tissues.

Generation of Mtch2 knock-out mice via homologous recombination—A targeting vector was constructed to knock out the first three exons of the 13 exons of Mtch2 (FIG. 21a). The targeting vector was transfected into R1 embryonic stem (ES) cells (derived from 129/ola mice), and neomycin resistant clones were selected. Southern blot analysis performed using the 5′ probe (FIG. 21b) or the 3′ probe (FIG. 21c) demonstrated the occurrence of homologues recombination in two clones. The homologous recombinant R1 clones were aggregated with tetraploid embryos and implanted into separate white-coated ICR foster mother mice. The first generation of black-coated mice were born, bred again to white ICR mice, and a second generation of Mtch2+/−animals was born. Homozygous Mtch2−/− offspring can result by intercrossing the Mtch2+/−animals.

Analysis and Discussion

BCL-2 family members are major regulators of the apoptotic process. The mechanisms by which these proteins regulate cell death are largely unknown, although it is thought that their function depends mostly on their ability to modulate the release of proteins from the inter-membrane space of the mitochondria.

Previous studies performed by the present inventors have shown that the addition of TNFα to FL5.12 cells resulted in the formation of a 45 kDa-tBID mitochondrial complex [Grinberg M, 2002 (Supra)]. The findings presented hereinabove demonstrate that the 45 kDa complex represents an association between tBID and the mitochondrial carrier homolog 2 (Mtch2), a novel and previously uncharacterized protein.

Mtch2 has several close relatives, which together form a “sister family” separate from the other mitochondrial carrier proteins (FIGS. 7a-d). All Mtch family members contain a single mitochondrial carrier domain (MCD), three transmembrane domains, and can be aligned across almost their entire length. Mtch proteins are thus a distinct subgroup of proteins related to mitochondrial carrier proteins (MCPs). In addition, the human presenilin-1-associated protein (PSAP, also known as human Mtch1) which exhibits 48% identity to Mtch2, is known to localize to the mitochondria and induce Cyt c release, caspase activation and apoptosis when overexpressed in 293T cells [Xu X, 2002 (Supra)].

Mtch2 is likely to participate in tBID-induced apoptosis, since in apoptotic cells tBID is recruited together with BAX to a relatively large resident complex (about 185 kDa) that includes Mtch2. Furthermore, overexpression of BCL-X_{L}, which inhibits the TNFα-induced release of Cyt c, partially inhibits the recruitment of both tBID and BAX to this resident complex. Inhibition of tBID is due to a direct interaction with BCL-X_{L}, since TNFα induces the formation of a cross-linked complex between tBID and BCL-X_{L} in FL5.12-BCL-X_{L} cells. Interestingly, a similar interaction between tBID and BCL-X_{L} also occurs in TNFα-treated FL5.12 parent cells (data not shown), but in these cells the recruitment of tBID and BAX to the Mtch2 resident complex is not effected (FIGS. 14a-f). Thus, at low levels of BCL-X_{L} the recruitment to the Mtch2 complex is not inhibited. On the other hand, at high levels of BCL-X_{L}, the recruitment to the Mtch2 complex is inhibited, but this inhibition seems to be partial since there the interaction between tBID and Mtch2 could be still detected (FIGS. 16a-d). Nevertheless, the fact that FL5.12-BCL-X_{L} cells do not release Cyt c in response to TNFα [Gross A, 1999, (Supra)] suggests that this partial inhibition of the recruitment of tBID and BAX to the Mtch2-resident complex is sufficient to inhibit Cyt c release.

Since Mtch2 is an integral membrane protein that is exposed on the surface of mitochondria, it is tempting to speculate that it might act to stably anchor and/or correctly position tBID in the membrane to enable the transient activation of BAX. In this respect, it was previously demonstrated that BAX oligomerization requires the presence of both tBID and a mitochondrial protein that is sensitive to proteinase K (Roucou, X., et al., 2002. Biochem. J. 368:915-21). The fact that tBID BH3 mutant (tBID-G94E) is capable of forming the 45 kDa complex [Grinberg M, 2002 (Supra)], but does not interact with BAX (Wang, K., et al., 1996. Genes Dev 10:2859-69) suggests that tBID could interact simultaneously with both Mtch2 and BAX.

BAX is a downstream effector of tBID, and its presence is essential for tBID-induced apoptosis. It is well established that tBID induces the dimerization/oligomerization of BAX; however, it is still not clear whether these oligomers can indeed form channels in the mitochondrial membrane of intact cells. Alternatively, BAX could regulate the activity of pre-existing channels and/or carriers, rather than form channels itself. Thus, it is possible that the role played by tBID involves the recruitment of BAX to the Mtch2 resident complex, which contains a channel/carrier that is activated by BAX.

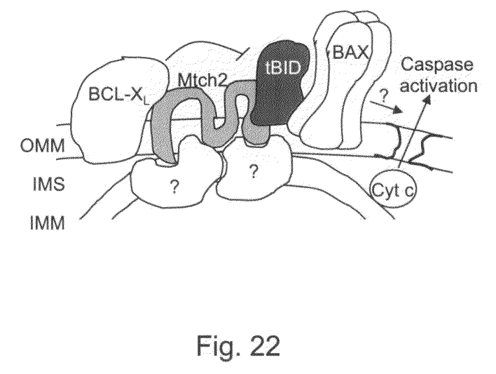

Altogether, the findings of the present invention demonstrate that tBID forms a stable interaction with a resident mitochondrial protein in cells signaled to die by TNFα. As is schematically illustrated in FIG. 22, Mtch2 resides in the OMM in a relatively large complex that might include IMM proteins. In response to TNFα, tBID and BAX are recruited to the Mtch2-resident complex. At the mitochondria, tBID induces the oligomerization of BAX, which results in the release of Cyt c and caspase activation. In cells overexpressing BCL-X_{L}, the TNFα-induced recruitment of tBID and BAX to the Mtch2-resident complex is partially inhibited, along with the inhibition of Cyt c release.

Using the RNAi approach the present inventors showed that downregulation of Mtch2 in HeLa cells results in acceleration of Fas- and TNF-α-induced apoptosis (FIG. 18c) and accelerated tBID-induced Cyt c release (FIG. 19b). In addition, in mitochondria prepared from Mtch2 downregulated HeLa cells there was less targeting of tBID as compared with control cells, and a significantly less formation of the 45 kDa complex (FIG. 19a). These results suggest that tBID negatively regulates the pro-survival activity of Mtch2 and therefore downregulation of Mtch2 (using e.g., RNAi) facilitates tBID-induced apoptosis in the mitochondria.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents, and patent applications and sequences identified by their accession numbers mentioned in this specification are herein incorporated in their entirety by reference into the specification, to the same extent as if each individual publication, patent, or patent application or sequence identified by its accession number was specifically and individually indicated to be incorporated herein by reference. In addition, citation or identification of any reference in this application shall not be construed as an admission that such reference is available as prior art to the present invention.

1.-50. (canceled)

51. A method of regulating apoptosis in a cell, the method comprising contacting the cell with an agent capable of modulating:
(i) an expression of an MCD-containing protein capable of tBID binding activity; and/or
(ii) said tBID binding activity of said MCD-containing protein, thereby regulating apoptosis in the cell.

52. The method of claim 51, wherein said regulating apoptosis is upregulating apoptosis.

53. The method of claim 52, wherein said agent is capable of decreasing said expression of said MCD-containing protein.

54. The method of claim 53, wherein said agent is as set forth in SEQ ID NO:27.

55. The method of claim 52, wherein said agent is capable of increasing said tBID binding activity of said MCD-containing protein.

56. The method of claim 51, wherein said regulating apoptosis is downregulating apoptosis.

57. The method of claim 56, wherein said agent is capable of increasing said expression of said MCD-containing protein.

58. The method of claim 56, wherein said agent is capable of decreasing said tBID binding activity of said MCD-containing protein.

59. The method of claim 58, wherein said agent is an antibody or a peptide.

60. The method of claim 51, wherein said MCD-containing protein is Mtch2.

61. The method of claim 60, wherein said Mtch2 is a human Mtch2 as set forth by SEQ ID NO:7.

62. A method of treating a pathology associated with disregulated apoptosis in a subject comprising administering to the subject an agent capable of modulating:
(i) an expression of an MCD-containing protein capable of tBID binding activity; and/or
(ii) said tBID binding activity of said MCD-containing protein, thereby treating the pathology.

63. The method of claim 62, wherein the disregulated apoptosis is characterized by abnormally low level of apoptosis and whereas said agent is capable of upregulating apoptosis.

64. The method of claim 63, wherein said agent is as set forth in SEQ ID NO:27.

65. The method of claim 62, wherein the disregulated apoptosis is characterized by abnormally high level of apoptosis and whereas said agent is capable downregulating apoptosis.

66. The method of claim 62, wherein said MCD-containing protein is Mtch2.

67. The method of claim 66, wherein said Mtch2 is a human Mtch2 as set forth by SEQ ID NO:7.

68. A pharmaceutical composition for regulating apoptosis comprising an agent capable of modulating:
(i) an expression of an MCD-containing protein capable of tBID binding activity; and/or
(ii) said tBID binding activity of said MCD-containing protein, and a pharmaceutical acceptable carrier.

69. The pharmaceutical composition of claim 68, wherein the regulating apoptosis is upregulating apoptosis.

70. The pharmaceutical composition of claim 69, wherein said agent is as set forth in SEQ ID NO:27.

71. The pharmaceutical composition of claim 68, wherein the regulating apoptosis is downregulating apoptosis.

72. The method of claim 68, wherein said MCD-containing protein is Mtch2.

73. The pharmaceutical composition of claim 72, wherein said Mtch2 is a human Mtch2 as set forth by SEQ ID NO:7.

74. A multicellular non-human organism comprising a genome which comprises a genetically modified Mtch2 gene being incapable of encoding a functional Mtch2 protein.

75. The multicellular organism of claim 74, wherein an integration of said genetically modified Mtch2 gene in said genome is induced postnatally.
